(19) United States
(12) Patent Application Publication
(10) Pub. No.: US 2010/0298278 A1
(43) Pub. Date: Nov. 25, 2010

(54) INHIBITORS OF BETA-SECRETASE FOR THE TREATMENT OF ALZHEIMER'S DISEASE

(76) Inventors: Christian Eickmeier, Mittelbiberach (DE); Klaus Fuchs, Mittelbiberach (DE); Niklas Heine, Biberach (DE); Stefan Peters, Biberach (DE); Cornelia Dorner-Ciossek, Ravensburg (DE); Sandra Handschuh, Biberach (DE); Herbert Nar, Ochsenhausen (DE); Klaus Klinder, Oggelshausen (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(21) Appl. No.: 12/063,270

(22) PCT Filed: Aug. 8, 2006
(86) PCT No.: PCT/EP06/65151

§ 371 Date: Feb. 8, 2008

(30) Foreign Application Priority Data
Aug. 11, 2005 (EP) 05017477.0

(51) Int. Cl. A61K 31/549; C07D 417/12; C07D 285/16; C07D 277/28; C07D 211/96; C07D 295/26; C07D 265/10; C07D 413/12; A61K 31/426; A61K 31/4453; A61K 31/5375; A61K 31/535; A61K 31/445; A61K 31/473; A61K 31/55; A61K 31/439; A61K 31/437; A61K 31/65; A61K 31/496; A61P 25/28; A61P 3/10; A61P 9/10; A61P 1/18; A61P 25/00

(52) U.S. Cl. 514/154; 544/8; 548/204; 546/269.7; 546/247; 544/159; 544/97; 514/222.5; 514/365; 514/331; 514/238.2; 514/228.8; 514/319; 514/297; 514/215; 514/294; 514/303; 514/254.11

(57) ABSTRACT

The invention relates to the substituted 1,2-ethylenediamines of general formula (I),

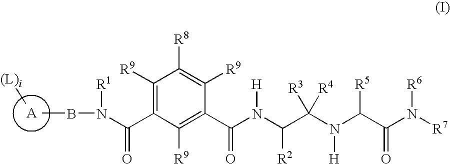

wherein the groups R¹ to R¹³, A, B, L and i are defined as in the description and the claims. The invention also relates to the use thereof in the treatment of Alzheimer's disease (AD) and similar diseases.

The present invention relates to substituted 1,2-ethylenediamines of general formula (I)

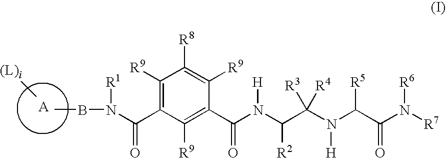

wherein the groups R¹ to R¹³, A, B, L and i are defined hereinafter, including the pharmacologically acceptable salts, diastereomers, enantiomers, racemates, hydrates and solvates thereof. The invention also relates to pharmaceutical compositions containing a compound of formula I according to the invention and the use of a compound according to the invention for preparing a pharmaceutical composition for the treatment and/or prevention of Alzheimer's disease (AD) and other diseases associated with abnormal processing of Amyloid Precursor Protein (APP) or aggregation of Abeta peptide, as well as diseases that can be treated or alleviated by inhibiting β-secretase. Corresponding diseases include MCI (“mild cognitive impairment”), trisomy 21 (Down's syndrome), cerebral amyloidangiopathy, degenerative dementias, hereditary cerebral haemorrhage with amyloidosis-Dutch type (HCHWA-D), Alzheimer's dementia with Lewy bodies, trauma, stroke, pancreatitis, inclusion body myositis (IBM), as well as other peripheral amyloidoses, diabetes and arteriosclerosis.

The compounds according to the invention also inhibit the aspartylprotease cathepsin D and are therefore suitable for suppressing the metastasisation of tumour cells.

This invention also relates to processes for preparing a pharmaceutical composition as well as a compound according to the invention.

### BACKGROUND TO THE INVENTION

EP 652 009 A1 describes inhibitors of aspartate protease which inhibit the production of beta-amyloid peptides in cell culture and in vivo.

WO 00/69262 discloses a beta-secretase and its use in assays for discovering potential active substances for the treatment of AD.

WO 01/00663 discloses memapsin 2 (human beta-secretase) and also a recombinant catalytically active enzyme. In addition, methods of identifying inhibitors of memapsin 2 are described.

WO 01/00665 discloses inhibitors of memapsin 2 for the treatment of AD.

WO 03/057721 discloses substituted aminocarboxamides for the treatment of AD.

WO 05/004802 discloses substituted benzyl-substituted N-alkyl-phenylcarboxamides for the treatment of AD.

At present there are no effective treatment methods capable of preventing, stopping or reversing AD.

### PROBLEM OF THE INVENTION

The problem of the present invention is therefore to provide new substituted 1,2-ethylenediamines which inhibit the cleaving of APP (Amyloid Precursor Protein) mediated by β-secretase.

The present invention also sets out to provide physiologically acceptable salts of the compounds according to the invention with inorganic or organic acids.

A further aim of the present invention is to provide pharmaceutical compositions that contain at least one compound according to the invention or a physiologically acceptable salt according to the invention, optionally together with one or more inert carriers and/or diluents.

The present invention further relates to pharmaceutical compositions containing one or more, preferably one active substance, which is selected from among the compounds according to the invention and/or the corresponding salts, as well as one or more, preferably one further active substance, optionally in addition to one or more inert carriers and/or diluents.

A further aim of this invention relates to the use of at least one of the compounds according to the invention for inhibiting β-secretase.

The invention also sets out to provide new pharmaceutical compositions that are suitable for the treatment or prevention of diseases or conditions that are associated with an abnormal processing of Amyloid Precursor Protein (APP) or aggregation of Abeta peptide.

A further aim of this invention is to provide new pharmaceutical compositions which are suitable for the treatment or prevention of diseases or conditions that can be influenced by inhibiting the β-secretase activity.

The invention also sets out to provide new pharmaceutical compositions which are suitable for the treatment and/or prevention of Alzheimer's disease (AD) as well as other diseases associated with an abnormal processing of APP or aggregation of Abeta peptide, as well as diseases that can be treated or prevented by inhibiting β-secretase, particularly AD.

In a further aspect this invention relates to a method of inhibiting the β-secretase activity.

Further aims of the present invention will become directly apparent to the skilled man from the foregoing remarks and those that follow.

### SUBJECT OF THE INVENTION

In a first aspect the present invention relates to substituted 1,2-ethylenediamines of general formula (I)

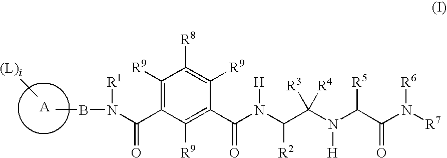

wherein
A denotes aryl or heteroaryl,
   wherein the group A, besides the groups L, may optionally be substituted by one or more fluorine atoms,
L in each case independently of one another denote hydrogen, fluorine, chlorine, bromine, iodine, hydroxy, carboxy, formyl, cyano, nitro, F₃C, HF₂C, FH₂C, C₁₋₆-alkyl, C₂₋₆-alkenyl, C₂₋₆-alkynyl, C₁₋₆-alkyl-S, C₁₋₆-alkyl-S—C₁₋₃-alkyl, C₃₋₇-cycloalkyl, C₃₋₇-cycloalkyl-C₁₋₆-alkyl, C₃₋₇-cycloalkyl-C₂₋₆-alkenyl, C₃₋₇-cycloalkyl-C₂₋₆-alkynyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, heterocyclyl, heterocyclyl-C₁₋₆-alkyl, heterocyclyl-C₂₋₆-alkenyl, heterocyclyl-C₂₋₆-alkynyl, aryl, aryl-C₁₋₆-alkyl, aryl-C₂₋₆-alkenyl, aryl-C₂₋₆-alkynyl, aryl-C₃₋₇-cycloalkyl, heteroaryl, heteroaryl-C₁₋₆-alkyl, heteroaryl-C₂₋₆-alkenyl, heteroaryl-C₂₋₆-alkynyl, heteroaryl-C₃₋₇-cycloalkyl, R¹³—O, R¹³—O—C₁₋₃-alkyl, (R¹²)₂N, (R¹²)₂N—CO, R¹²—CO—(R¹²)N, (R¹²)₂N—CO—(R¹²)N, R¹²—SO₂—(R¹²)N, (R¹²)₂N—SO₂ or C₁₋₆-alkyl-SO₂,
   wherein the above mentioned groups may optionally be substituted independently of one another by one or more groups selected from among fluorine, chlorine, bromine, iodine, hydroxy, oxo, carboxy, formyl, cyano, nitro, F₃C, HF₂C, FH₂C, hydroxy-C₁₋₆-alkyl, C₁₋₃-alkyl, C₁₋₆-alkoxy, (R¹²)₂N, (R¹²)₂N—C₁₋₃-alkyl, (R¹²)₂N—CO— and HOSO₂—,
i denotes 0, 1, 2 or 3,
B denotes a C₁₋₄-alkylene bridge,
   while the C₁₋₄-alkylene bridge may optionally be substituted by one or more groups selected from among fluorine, chlorine, bromine, iodine, hydroxy, oxo, carboxy, cyano, nitro, F₃C, HF₂C, FH₂C, C₁₋₄-alkyl, C₁₋₆-alkyl-S—C₁₋₃-alkyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, aryl-C₃₋₇-cycloalkyl, heteroaryl, heteroaryl-C₁₋₃-alkyl, heteroaryl-C₃₋₇-cycloalkyl, R¹³—O, (R¹²)₂N—SO₂, (R¹²)₂N, (R¹²)₂N—C₁₋₃-alkyl, (R¹²)₂N—CO, R¹²—SO₂, R¹²—CO(R¹²)N, R¹²—SO₂(R¹²)N, (R¹²)₂N—SO₂, R¹²—CO— and R¹²—SO—, and
   wherein two C₁₋₄-alkyl groups bound to the same carbon atom of the C₁₋₄-alkylene bridge may be joined together, forming a C₃₋₇-cycloalkyl group, and
   wherein the above mentioned C₁₋₄-alkyl groups and the C₃₋₇-cycloalkyl group formed from the C₁₋₄-alkyl groups may optionally be substituted independently of one another by one or more groups selected from among fluorine, chlorine, bromine, iodine, hydroxy, oxo, carboxy, formyl, cyano, nitro, F₃C, C₁₋₃-alkyl, C₁₋₃-alkoxy, R¹³—O—C₁₋₃-alkyl, R¹²—CO(R¹²)N, R¹²—SO₂(R¹²)N, (R¹²)₂N, (R¹²)₂N—C₁₋₃-alkyl, (R¹²)₂N—CO, (R¹²)₂N—SO₂— and HOSO₂—,
R¹ denotes hydrogen, C₁₋₆-alkyl, C₂₋₆-alkenyl, C₂₋₆-alkynyl, C₃₋₇-cycloalkyl, C₃₋₇-cycloalkyl-C₁₋₆-alkyl, C₃₋₇-cycloalkyl-C₂₋₆-alkenyl, C₃₋₇-cycloalkyl-C₂₋₆-alkynyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, heterocyclyl, heterocyclyl-C₁₋₆-alkyl, heterocyclyl-C₂₋₆-alkenyl, heterocyclyl-C₂₋₆-alkynyl, aryl, aryl-C₁₋₆-alkyl, aryl-C₂₋₆-alkenyl, aryl-C₂₋₆-alkynyl, aryl-C₃₋₇-cycloalkyl, heteroaryl, heteroaryl-C₁₋₆-alkyl, heteroaryl-C₂₋₆-alkenyl, heteroaryl-C₂₋₆-alkynyl or heteroaryl-C₃₋₇-cycloalkyl,
   wherein the above mentioned groups may optionally be substituted independently of one another by one or more groups selected from among fluorine, chlorine, bromine, iodine, hydroxy, oxo, carboxy, formyl, cyano, nitro, F₃C, C₁₋₃-alkyl, C₁₋₃-alkoxy, hydroxy-C₁₋₆-alkyl, (R¹²)₂N, (R¹²)₂N—C₁₋₃-alkyl, (R¹²)₂N—CO, (R¹²)₂N—SO₂, R¹²—CO—(R¹²)N, R¹²—SO₂(R¹²)N— and HOSO₂—,
R² denotes C₁₋₆-alkyl, C₂₋₆-alkenyl, C₂₋₆-alkynyl, C₁₋₆-alkoxy-C₁₋₃-alkyl, C₁₋₆-alkyl-S—C₁₋₃-alkyl, C₃₋₇-cycloalkyl, C₃₋₇-cycloalkyl-C₁₋₃-alkyl, C₃₋₇-cycloalkyl-C₂₋₃-alkenyl, C₃₋₇-cycloalkyl-C₂₋₃-alkynyl, C₃₋₇-cycloalkenyl, C₃₋₇-cycloalkenyl-C₁₋₃-alkyl, C₃₋₇-cycloalkenyl-C₂₋₃-alkenyl, C₃₋₇-cycloalkenyl-C₂₋₃-alkynyl, heterocyclyl, heterocyclyl-C₁₋₃-alkyl, heterocyclyl-C₂₋₃-alkenyl, heterocyclyl-C₂₋₃-alkynyl, aryl, aryl-C₁₋₃-alkyl, aryl-C₂₋₃-alkenyl, aryl-C₂₋₃-alkynyl, aryl-C₃₋₇-cycloalkyl, heteroaryl, heteroaryl-C₁₋₃-alkyl, heteroaryl-C₂₋₃-alkenyl, heteroaryl-C₂₋₃-alkynyl or heteroaryl-C₃₋₇-cycloalkyl,
   wherein the above mentioned groups may optionally be substituted independently of one another by one or more groups selected from among fluorine, chlorine, bromine, iodine, F₃C, F₃C, HF₂C, FH₂C-hydroxy, oxo, carboxy, formyl, cyano, nitro, (R¹²)₂N, (R¹²)₂N—C₁₋₃-alkyl, HOSO₂, C₁₋₃-alkyl, C₁₋₆-alkyl-S—C₁₋₃-alkyl, (R¹²)₂N—SO₂, R¹²—CO—(R¹²)N, R¹²—SO₂(R¹²)N, (R¹²)₂N—C₁₋₃-alkyl, (R¹²)₂N—CO, R¹³—O— and R¹³—O—C₁₋₃-alkyl-,
R³, R⁴ in each case independently of one another denote hydrogen, C₁₋₆-alkyl, fluorine, F₃C, HF₂C or FH₂C,
R⁵ denotes hydrogen, C₁₋₆-alkyl, C₂₋₆-alkenyl, C₂₋₆-alkynyl, C₃₋₇-cycloalkyl, C₃₋₇-cycloalkyl-C₁₋₄-alkyl, C₃₋₇-cycloalkyl-C₂₋₄-alkenyl, C₃₋₇-cycloalkyl-C₂₋₄-alkynyl, C₃₋₇-cycloalkenyl, C₃₋₇-cycloalkenyl-C₁₋₄-alkyl, C₃₋₇-cycloalkenyl-C₂₋₄-alkenyl, C₃₋₇-cycloalkenyl-C₂₋₄-alkynyl, heterocyclyl, heterocyclyl-C₁₋₄-alkyl, heterocyclyl-C₂₋₄-alkenyl, heterocyclyl-C₂₋₄-alkynyl, aryl, aryl-C₁₋₄-alkyl, aryl-C₂₋₄-alkenyl, aryl-C₂₋₄-alkynyl, aryl-C₃₋₇-cycloalkyl, heteroaryl, heteroaryl-C₁₋₄-alkyl, heteroaryl-C₂₋₄-alkenyl, heteroaryl-C₂₋₄-alkynyl or heteroaryl-C₃₋₇-cycloalkyl,
   wherein the above mentioned groups may optionally be substituted independently of one another by one or more groups selected from among fluorine, chlorine, bromine, iodine, hydroxy, oxo, carboxy, formyl, cyano, nitro, C₁₋₃-alkyl, C₁₋₆-alkoxy, C₁₋₃-alkyl-S, aryl, heteroaryl, heteroaryl-C₁₋₃-alkyl, aryl-C₁₋₆-alkyl, R¹²—CO—(R¹²)N, R¹²—SO₂(R¹²)N—(R¹²)₂N—SO₂, (R¹²)₂N, (R¹²)₂N—C₁₋₃-alkyl, (R¹²)₂N—CO— and HOSO₂—,
R⁶ denotes hydrogen, C₁₋₆-alkyl, C₂₋₆-alkenyl, C₂₋₆-alkynyl, C₃₋₇-cycloalkyl, C₃₋₇-cycloalkyl-C₁₋₆-alkyl, C₃₋₇-cycloalkyl-C₂₋₆-alkenyl, C₃₋₇-cycloalkyl-C₂₋₆-alkynyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, heterocyclyl, heterocyclyl-C₁₋₆-alkyl, heterocyclyl-C₂₋₆-alkenyl, heterocyclyl-C₂₋₆-alkynyl, aryl, aryl-C₁₋₆-alkyl, aryl-C₂₋₆-alkenyl, aryl-C₂₋₆-alkynyl, aryl-C₃₋₇-cycloalkyl, heteroaryl, heteroaryl-C₁₋₆-alkyl, heteroaryl-C₂₋₆-alkenyl, heteroaryl-C₂₋₆-alkynyl or heteroaryl-C₃₋₇-cycloalkyl,
   wherein the above mentioned groups may optionally be substituted independently of one another by one or more groups selected from among fluorine, chlorine, bromine, iodine, hydroxy, oxo, carboxy, formyl, cyano, nitro, C₁₋₃-alkyl, heterocyclyl, heterocyclyl-C₁₋₃-alkyl, R¹³—O, R¹³—O—C₁₋₃-alkyl, aryl, heteroaryl, heteroaryl-C₁₋₃-alkyl, aryl-C₁₋₆-alkyl, (R¹²)₂N, (R¹²)₂N—C₁₋₃-alkyl, (R¹²)₂N—CO, (R¹²)₂N—CO—N(R¹²), (R¹²)₂N—SO₂— and HOSO₂—,
R⁷ denotes hydrogen, C₁₋₆-alkyl, C₂₋₆-alkenyl, C₂₋₆-alkynyl, C₁₋₆-alkoxy-C₁₋₃-alkyl, C₃₋₇-cycloalkyl, C₃₋₇-cycloalkyl-C₁₋₃-alkyl, heterocyclyl-C₁₋₃-alkyl, aryl, aryl-C₁₋₃-alkyl, heteroaryl or heteroaryl-C₁₋₃-alkyl,
   wherein the above mentioned groups may optionally be substituted independently of one another by one or more groups selected from among fluorine, chlorine, bromine, iodine, cyano, hydroxy, C₁₋₃-alkyl, C₁₋₆-alkoxy and (R¹²)₂N,
R⁸ denotes C₁₋₆-alkyl, C₂₋₆-alkenyl, C₂₋₆-alkynyl, C₃₋₇-cycloalkyl, C₃₋₇-cycloalkyl-C₁₋₃-alkyl, heterocyclyl, heterocyclyl-C₁₋₃-alkyl, C₃₋₇-cycloalkyl-C₂₋₄-alkenyl, heterocyclyl-C₂₋₄-alkenyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, aryl-C₁₋₃-alkyl, heteroaryl, heteroaryl-C₁₋₃-alkyl, aryl-C₂₋₃-alkenyl, heteroaryl-C₂₋₃-alkenyl, aryl-C₂₋₃-alkynyl, aryl-C₃₋₇-cycloalkyl, heteroaryl-C₂₋₃-alkynyl, heteroaryl-C₃₋₇-cycloalkyl, C₃₋₇-cycloalkyl-C₂₋₄-alkynyl, heterocyclyl-C₂₋₄-alkynyl, R¹³—O, R¹³—O—C₁₋₃-alkyl, R¹⁰—SO₂—(R¹¹)N or R¹⁰—CO—(R¹¹)N,
   wherein the C₁₋₆-alkyl group and the C₃₋₇-cycloalkyl group may optionally be substituted independently of one another by one or more groups selected from among C₁₋₆-alkyl, fluorine, chlorine, bromine, hydroxy, oxo, carboxy, formyl, nitro, C₂₋₇-alkenyl, C₂₋₇-alkynyl, C₁₋₆-alkyl-S, C₁₋₆-alkyl-S—C₁₋₃-alkyl, C₃₋₆-cycloalkyl, C₃₋₇-cycloalkyl-C₁₋₆-alkyl, aryl, aryl-C₁₋₆-alkyl, heterocyclyl, heterocyclyl-C₁₋₆-alkyl, heteroaryl, heteroaryl-C₁₋₆-alkyl, R¹³—O, R¹³—O—CO, R¹³—CO, R¹³—O—CO—(R¹²)N, (R¹²)₂N—CO—O, R¹³—O—C₁₋₃-alkyl, (R¹²)₂N, (R¹²)₂N—CO, R¹²—CO—(R¹²)N, (R¹²)₂N—CO—(R¹²)N, (R¹²)₂N—SO₂, (R¹²)₂N—SO₂—(R¹²)N, R¹²—SO₂, F₃C, HF₂C, FH₂C, F₃C—O, HF₂C—O, FH₂C—O— or R¹²—SO₂—(R¹²)N—, and
   wherein the above mentioned C₂₋₆-alkenyl, C₂₋₆-alkynyl, C₃₋₇-cycloalkyl-C₁₋₃-alkyl, heterocyclyl, heterocyclyl-C₁₋₃-alkyl, C₃₋₇-cycloalkyl-C₂₋₄-alkenyl, heterocyclyl-C₂₋₄-alkenyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, aryl-C₁₋₃-alkyl, heteroaryl, heteroaryl-C₁₋₃-alkyl, aryl-C₂₋₃-alkenyl, heteroaryl-C₂₋₃-alkenyl, aryl-C₂₋₃-alkynyl, aryl-C₃₋₇-cycloalkyl, heteroaryl-C₂₋₃-alkynyl, heteroaryl-C₃₋₇-cycloalkyl, C₃₋₇-cycloalkyl-C₂₋₄-alkynyl and heterocyclyl-C₂₋₄-alkynyl groups may optionally be substituted independently of one another by one or more groups selected from among C₁₋₆-alkyl, fluorine, chlorine, bromine, hydroxy, oxo, carboxy, formyl, cyano, nitro, C₂₋₆-alkenyl, C₂₋₆-alkynyl, C₁₋₆-alkyl-S, C₁₋₆-alkyl-S—C₁₋₃-alkyl, C₃₋₇-cycloalkyl, C₃₋₇-cycloalkyl-C₁₋₆-alkyl, aryl, aryl-C₁₋₆-alkyl, heterocyclyl, heterocyclyl-C₁₋₆-alkyl, heteroaryl, heteroaryl-C₁₋₆-alkyl, R¹³—O, R¹³—O—CO, R¹³—CO, R¹³—O—CO—(R¹²)N, (R¹²)₂N—CO—O, R¹³—O—C₁₋₃-alkyl, (R¹²)₂N, (R¹²)₂N—CO, R¹²—CO—(R¹²)N, (R¹²)₂N—CO—(R¹²)N, (R¹²)₂N—SO₂, (R¹²)₂N—SO₂—(R¹²)N, R¹²—SO₂, F₃C, HF₂C, FH₂C, F₃C—O, HF₂C—O, FH₂C—O— or R¹²—SO₂—(R¹²)N,
R⁹ in each case independently of one another denote hydrogen, fluorine, chlorine, bromine, iodine, C₁₋₃-alkyl, R¹³—O or (R¹²)₂N—,
   wherein the above mentioned C₁₋₃-alkyl group may optionally be substituted by one or more fluorine atoms,
R¹⁰ denotes C₂₋₆-alkenyl, C₂₋₆-alkynyl, C₃₋₇-cycloalkyl-C₂₋₄-alkenyl, aryl-C₂₋₃-alkenyl, heteroaryl-C₂₋₃-alkenyl, heterocyclyl-C₂₋₄-alkenyl, aryl-C₂₋₃-alkynyl, C₃₋₇-cycloalkyl-C₂₋₄-alkynyl, heterocyclyl-C₂₋₄-alkynyl-heteroaryl-C₂₋₃-alkynyl, C₃₋₇-cycloalkyl, C₃₋₇-cycloalkyl-C₁₋₃-alkyl, heterocyclyl, heterocyclyl-C₁₋₃-alkyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, aryl-C₂₋₃-alkyl, aryl-C₃₋₇-cycloalkyl, heteroaryl, heteroaryl-C₁₋₃-alkyl, heteroaryl-C₃₋₇-cycloalkyl or (R¹²)₂N,
   wherein the above mentioned groups may optionally be substituted independently of one another by one or more groups selected from among fluorine, chlorine, bromine, hydroxy, oxo, carboxy, formyl, cyano, nitro, C₁₋₃-alkyl, heterocyclyl, heterocyclyl-C₁₋₃-alkyl, R¹³—O, R¹³—O—C₁₋₃-alkyl, R¹²—CO(R¹²)N, R¹²—SO₂(R¹²)N, (R¹²)₂N, (R¹²)₂N—C₁₋₃-alkyl and (R¹²)₂N—CO,
R¹¹ denotes C₂₋₆-alkenyl, C₂₋₆-alkynyl, C₃₋₇-cycloalkyl-C₁₋₃-alkyl, aryl-C₂₋₃-alkyl, heterocyclyl, heterocyclyl-C₁₋₃-alkyl, heteroaryl or heteroaryl-C₁₋₃-alkyl,
   wherein the above mentioned groups may optionally be substituted independently of one another by one or more groups selected from among fluorine, chlorine, bromine, hydroxy, oxo, carboxy, formyl, cyano, nitro, C₁₋₃-alkyl, heterocyclyl, heterocyclyl-C₁₋₃-alkyl, R¹³—O, R¹³—O—C₁₋₃-alkyl, (R¹²)₂N, (R¹²)₂N—C₁₋₃-alkyl- and R¹²CO—,
or
R¹⁰ and R¹¹ together form a C₂₋₆-alkylene bridge, so that a heterocyclic ring is formed with the inclusion of the nitrogen atom linked to R¹¹ and the SO₂— or CO— group linked to R¹⁰,
   wherein one or two —CH₂ groups of the C₂₋₆-alkylene bridge may be replaced independently of one another by O, S or —N(R¹²)— such that in each case two O, S or N atoms or an O and an S atom are not directly connected to one another, and
   wherein the C atoms of the above mentioned C₂₋₆-alkylene bridge may optionally be substituted by one or more groups selected from among fluorine, chlorine, bromine, hydroxy, carboxy, formyl, cyano, F₃C, C₁₋₆-alkyl, C₁₋₆-alkoxy, oxo and nitro,
R¹² in each case independently of one another denote hydrogen, C₁₋₆-alkyl, C₁₋₆-alkoxy-C₁₋₃-alkyl, C₃₋₆-cycloalkyl, C₃₋₆-cycloalkyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl, wherein
   two C₁₋₆-alkyl groups bound to the same nitrogen atom may together form a C₂₋₆-alkylene bridge, so that a heterocyclic ring is formed with the inclusion of the nitrogen atoms linked to the groups R¹²,
   while a —CH₂ group of the C₂₋₆-alkylene bridge may be replaced by O, S or —N(R¹³)—, and
   wherein the above mentioned groups and the heterocyclic ring may optionally be substituted independently of one another by one or more groups selected from among fluorine, chlorine, bromine, iodine, hydroxy, oxo, carboxy, formyl, cyano, nitro, C₁₋₃-alkyl, hydroxy-C₁₋₃-alkyl, C₁₋₃-alkoxy, (R¹³)₂N—CO or (R¹³)₂N—, and
R¹³ in each case independently of one another denote hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl,
   wherein the above mentioned groups may optionally be substituted independently of one another by one or more groups selected from among fluorine, chlorine, bromine, iodine, hydroxy, oxo, carboxy, formyl, cyano, nitro, C₁₋₃-alkyl- and C₁₋₃-alkoxy-,
      the pharmacologically acceptable salts, diastereomers, enantiomers, racemates, hydrates and solvates thereof.

The compounds of general formula (I) according to the invention and the physiologically acceptable salts thereof have valuable pharmacological properties, particularly an inhibiting effect on β-secretase activity, particularly the β-secretase mediated cleaving of APP.

In view of the inhibitory properties of the compounds according to the invention on the Cathepsin D activity, the compounds are also suitable for suppressing the metastasisation of tumour cells.

The present invention also relates to the physiologically acceptable salts of the compounds according to the invention with inorganic or organic acids.

Therefore in another aspect the invention also relates to the use of the compounds according to the invention, including the physiologically acceptable salts thereof, as medicaments.

The invention further relates to pharmaceutical compositions containing at least one compound according to the invention or a physiologically acceptable salt according to the invention, optionally together with one or more inert carriers and/or diluents.

This invention further relates to pharmaceutical compositions, containing one or more, preferably one active substance which is selected from among the compounds according to the invention and/or the corresponding salts, as well as one or more, preferably one active substance, for example selected from among beta-secretase inhibitors; gamma-secretase inhibitors; amyloid aggregation inhibitors such as e.g. Alzhemed; directly or indirectly acting neuroprotective substances; antioxidants such as e.g. Vitamin E or ginkgolides; anti-inflammatory substances such as e.g. Cox inhibitors, NSAIDs with additionally or only Aβ lowering properties; HMG-CoA reductase inhibitors (statins); acetylcholinesterase inhibitors such as donepezil, rivastigmine, tacrine, galantamine; NMDA receptor antagonists such as e.g. memantine; AMPA agonists; substances that modulate the concentration or release of neurotransmitters such as NS-2330; substances that induce the secretion of growth hormone such as ibutamoren mesylate and capromorelin; CB-1 receptor antagonists or inverse agonists; antibiotics such as minocycline or rifampicin; PDE-IV and PDE-IX inhibitors, GABA_{A} inverse agonists, nicotine agonists, histamine H3 antagonists, 5 HT-4 agonists or partial agonists, 5HT-6 antagonists, a2-adrenoreceptor antagonists, muscarinic M1 agonists, muscarinic M2 antagonists, metabotropic glutamate-receptor 5 positive modulators, as well as other substances that modulate receptors or enzymes in a manner such that the efficacy and/or safety of the compounds according to the invention is increased and/or unwanted side effects are reduced, optionally together with one or more inert carriers and/or diluents.

This invention further relates to pharmaceutical compositions, containing one or more, preferably one active substance, which is selected from among the compounds according to the invention and/or the corresponding salts, as well as one or more, preferably one active substance, selected from among Alzhemed, Vitamin E, ginkgolides, donepezil, rivastigmine, tacrine, galantamine, memantine, NS-2330, ibutamoren mesylate, capromorelin, minocycline and/or rifampicin, optionally together with one or more inert carriers and/or diluents.

This invention further relates to the use of at least one of the compounds according to the invention for inhibiting β-secretase.

This invention also relates to the use of at least one compound according to the invention or a physiologically acceptable salt of such a compound for preparing a pharmaceutical composition which is suitable for the treatment or prevention of diseases or conditions that are associated with abnormal processing of Amyloid Precursor Protein (APP) or aggregation of Abeta peptide.

This invention also relates to the use of at least one compound according to the invention or a physiologically acceptable salt of such a compound for preparing a pharmaceutical composition which is suitable for the treatment or prevention of diseases or conditions that can be influenced by inhibiting the β-secretase activity.

This invention further relates to the use of at least one compound according to the invention or a pharmaceutical composition according to the invention for preparing a pharmaceutical composition that is suitable for the treatment and/or prevention of Alzheimer's disease (AD) and other diseases associated with abnormal processing of Amyloid Precursor Protein (APP) or aggregation of Abeta peptide, as well as diseases that can be treated or alleviated by inhibiting β-secretase, particularly AD. Corresponding diseases include MCI (“mild cognitive impairment”), trisomy 21 (Down's syndrome), cerebral amyloidangiopathy, degenerative dementias, hereditary cerebral haemorrhage with amyloidosis-Dutch type (HCHWA-D), Alzheimer's dementia with Lewy bodies, trauma, stroke, pancreatitis, inclusion body myositis (IBM), as well as other peripheral amyloidoses, diabetes and arteriosclerosis.

This invention further relates to a method of inhibiting β-secretase activity, characterised in that β-secretase is brought into contact with an inhibitory amount of one of the compounds according to the invention.

Further subjects of the invention will become apparent to the skilled man in an obvious manner from the foregoing and following description of the invention.

### DETAILED DESCRIPTION OF THE INVENTION

Unless otherwise stated, the groups, residues and substituents R¹ to R¹³, A, B, L and i have the meanings given hereinbefore and hereinafter.

If residues, substituents or groups occur more than once in a compound, they may have the same or different meanings.

In a preferred embodiment of the compounds of the present invention the group

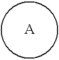

denotes a phenyl ring or a 5- or 6-membered aromatic heteroaryl group which contains 1, 2 or 3 heteroatoms selected from among N, O and S.

In another preferred embodiment the group

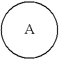

has the following meanings:

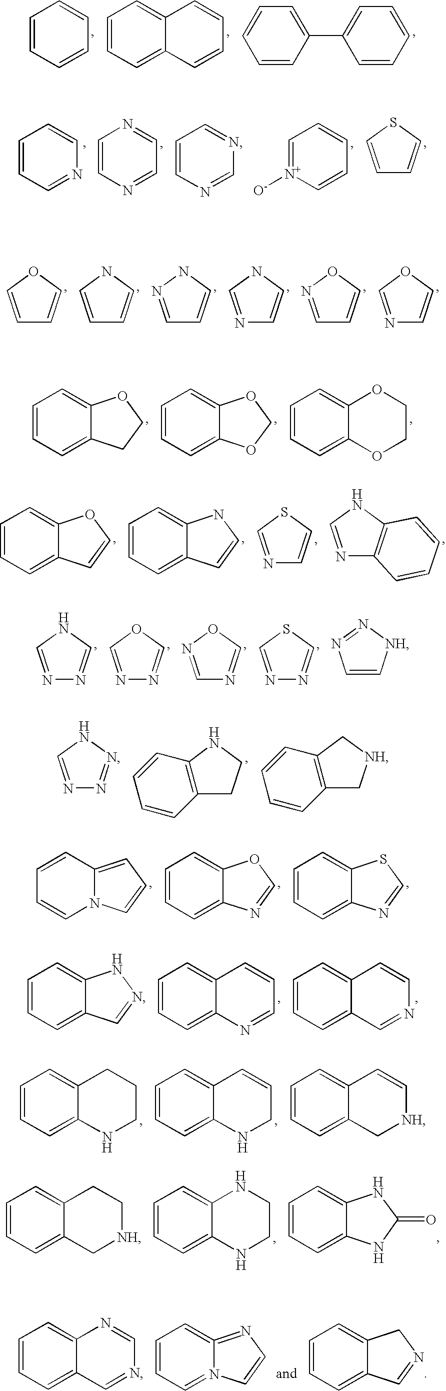

In a more preferred embodiment of the compounds of the present invention the group

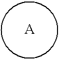

denotes a 5- or 6-membered aromatic heteroaryl group which contains 1 or 2 heteroatoms selected from among N, O and S, wherein at most one O or S atom may be present.

In einer particularly preferred embodiment the group

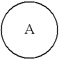

denotes a phenyl, thienyl, thiazolyl, pyrazolyl or a pyridyl group, wherein the phenyl, the thienyl, particularly the 3-thienyl, the thiazolyl, particularly the 2-thiazolyl and the pyridyl group, particularly the 2-pyridyl and the 3-pyridyl group, are particularly preferred.

Preferably the substituent L in each case independently denotes hydrogen, fluorine, chlorine, bromine, iodine, hydroxy, carboxy, cyano, nitro, F₃C, HF₂C, FH₂C, C₁₋₆-alkyl, C₂₋₆-alkenyl, C₂₋₆-alkynyl, C₃₋₇-cycloalkyl, C₃₋₇-cycloalkyl-C₁₋₃-alkyl, aryl, aryl-C₁₋₃-alkyl, heterocyclyl, heterocyclyl-C₁₋₃-alkyl, heteroaryl, heteroaryl-C₁₋₃-alkyl, R¹³—O, R¹³—O—C₁₋₃-alkyl, (R¹²)₂N, (R¹²)₂N—CO, R¹²—CO—(R¹²)N, (R¹²)₂N—CO—(R¹²)N, (R¹²)₂N—SO₂, R¹²—SO₂—(R¹²)N or C₁₋₃-alkyl-SO₂, wherein the above mentioned groups may optionally be substituted independently of one another by one or more groups selected from among fluorine, chlorine, bromine, hydroxy, oxo, carboxy, cyano, nitro, F₃C, HF₂C, FH₂C, hydroxy-C₁₋₃-alkyl, C₁₋₃-alkyl, C₁₋₃-alkoxy, (R¹²)₂N, (R¹²)₂N—C₁₋₃-alkyl- and (R¹²)₂N—CO—.

Particularly preferably the substituent L in each case independently denotes hydrogen, fluorine, chlorine, bromine, cyano, hydroxy, C₁₋₆-alkyl, C₁₋₆-alkoxy, C₃₋₇-cycloalkyl, C₃₋₇-cycloalkyl-C₁₋₃-alkyl, phenyl, (R¹²)₂N, (R¹²)₂N—CO, R¹²—CO—(R¹²)N, (R¹²)₂N—CO—(R¹²)N, R¹²—SO₂—(R¹²)N or (R¹²)₂N—SO₂, wherein the above mentioned groups may optionally be substituted by one or more fluorine atoms.

Most particularly preferred meanings for the substituent L are in each case independently of one another hydrogen, fluorine, chlorine, bromine, hydroxy, C₁₋₄-alkyl or C₁₋₄-alkoxy, wherein the above mentioned groups may optionally be substituted by one or more fluorine atoms.

Particularly preferred meanings for the substituent L are in each case independently of one another hydrogen, fluorine, chlorine, trifluoromethyl, trifluoromethoxy, methyl and methoxy.

Preferably the index i may assume the values 0, 1 or 2. In particularly preferred embodiments the value of the index i is 0 or 1.

In a preferred embodiment of the compounds according to the invention the group B denotes a C₁₋₄-alkylene bridge, which may optionally be substituted independently of one another by one or more groups selected from among fluorine, hydroxy, carboxy, cyano, nitro, F₃C, HF₂C, FH₂C, C₁₋₄-alkyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl, heteroaryl-C₁₋₃-alkyl, R¹³—O, (R¹²)₂N—SO₂— and (R¹²)₂N—, and wherein two C₁₋₄-alkyl groups bound to the same carbon atom of the C₁₋₄-alkylene bridge may be joined together, forming a C₃₋₇-cycloalkyl group, and wherein the above mentioned groups and the C₃₋₇-cycloalkyl group formed from the C₁₋₄-alkyl groups may optionally be substituted independently of one another by one or more groups selected from among fluorine, chlorine, bromine, hydroxy, carboxy, cyano, F₃C, C₁₋₃-alkyl, C₁₋₃-alkoxy and R¹³—O—C₁₋₃-alkyl.

Particularly preferably the group B denotes a C₁₋₄-alkylene bridge, while the C₁₋₄-alkylene bridge may optionally be substituted independently of one another by one or more groups selected from among fluorine, C₁₋₄-alkyl, phenyl or benzyl, and wherein two C₁₋₄-alkyl groups bound to the same carbon atom of the C₁₋₄-alkylene bridge may be joined together forming a C₃₋₆-cycloalkyl group, and wherein the above mentioned groups and the C₃₋₆-cycloalkyl group formed from the C₁₋₄-alkyl groups may optionally be substituted independently of one another by one or more groups selected from among fluorine, hydroxy and C₁₋₃-alkoxy.

In a most particularly preferred embodiment B is a C₁₋₂-alkylene bridge, wherein the C₁₋₂-alkylene bridge may optionally be substituted by one or more C₁₋₄-alkyl groups, and wherein two C₁₋₄-alkyl groups bound to the same carbon atom of the C₁₋₂-alkylene bridge may be joined together to form a cyclopropyl group, and wherein one or more hydrogen atoms of the above mentioned C₁₋₂-alkylene bridge and/or the C₁₋₄-alkyl groups and/or the cyclopropyl group formed therefrom may optionally be replaced by one or more fluorine atoms.

Particularly preferred are those compounds according to the invention, wherein the group B is selected from among

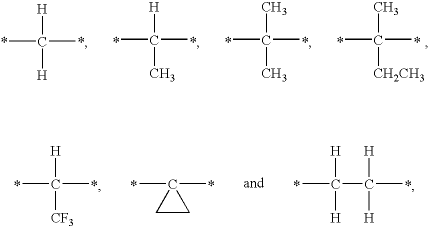

wherein one or more hydrogen atoms may optionally be replaced by fluorine.

Particularly preferred are those compounds according to the invention, wherein the group B is selected from among

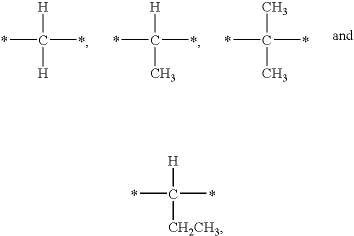

wherein one or more hydrogen atoms may optionally be replaced by fluorine.

Another preferred embodiment encompasses those compounds according to the invention wherein the partial formula (II)

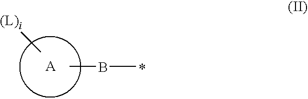

is selected from among

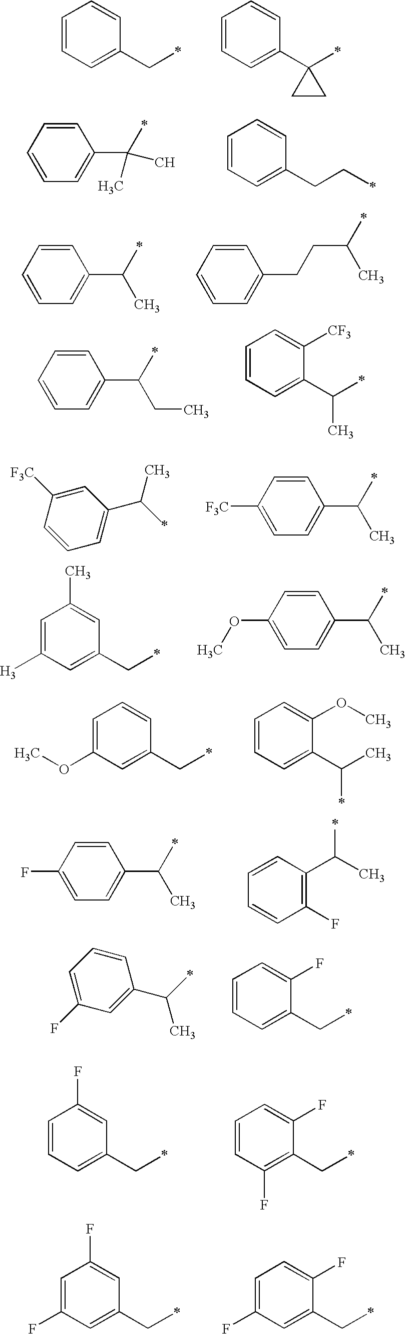

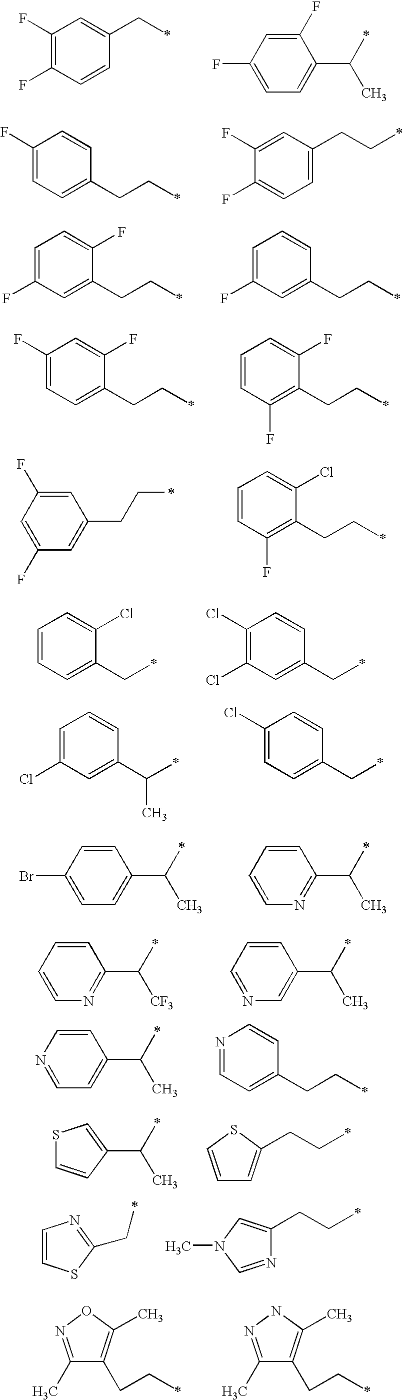

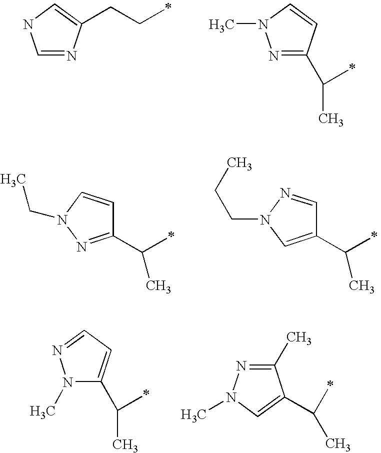

In the compounds of formula (I) according to the invention the group R¹ is preferably selected from among hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl and heteroaryl-C₁₋₃-alkyl,

wherein the above mentioned groups may optionally be substituted independently of one another by one or more groups selected from among fluorine, chlorine, bromine, hydroxy, carboxy, cyano, nitro, F₃C, C₁₋₃-alkyl, C₁₋₃-alkoxy- and hydroxy-C₁₋₃-alkyl.

Particularly preferred are the groups R¹ selected from among hydrogen, C₁₋₄-alkyl, C₃₋₄-alkenyl, C₃₋₆-cycloalkyl- and C₃₋₆-cycloalkyl-C₁₋₃-alkyl wherein the above mentioned groups may optionally be substituted independently of one another by one or more groups selected from among fluorine, hydroxy and C₁₋₃-alkoxy.

Most particularly preferred are the groups R¹ selected from among hydrogen and C₁₋₄-alkyl, wherein the C₁₋₄-alkyl group may be substituted by one or more fluorine atoms.

Particularly preferred are those compounds according to the invention wherein R¹ is hydrogen.

In the compounds according to the invention of formula (I) the group R² is preferably selected from among C₁₋₆-alkyl, C₂₋₆-alkenyl, C₂₋₆-alkynyl, C₁₋₆-alkoxy-C₁₋₃-alkyl, C₁₋₆-alkyl-S—C₁₋₃-alkyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl and heteroaryl-C₁₋₃-alkyl, wherein the above mentioned groups may optionally be substituted independently of one another by one or more groups selected from among fluorine, chlorine, bromine, iodine, F₃C, HF₂C, FH₂C, hydroxy, carboxy, cyano, nitro, C₁₋₃-alkyl, (R¹²)₂N, (R¹²)₂N—SO₂, R¹²—CO—(R¹²)N, R¹²—SO₂(R¹²)N, (R¹²)₂N—C₁₋₃-alkyl, (R¹²)₂N—CO, R¹³—O— and R¹³—O—C₁₋₃-alkyl.

Particularly preferred groups R² are groups selected from among C₁₋₆-alkyl, C₂₋₆-alkynyl, C₃₋₆-cycloalkyl-C₁₋₃-alkyl, heterocyclyl-C₁₋₃-alkyl, phenyl, phenyl-C₁₋₃-alkyl, heteroaryl and heteroaryl-C₁₋₃-alkyl, wherein by the above mentioned heteroaryl groups are meant 5- or 6-membered aromatic heteroaryl groups which contain 1, 2 or 3 heteroatoms selected from among N, O and S and wherein the above mentioned groups may optionally be substituted independently of one another by one or more groups selected from among fluorine, chlorine, bromine, iodine, cyano, hydroxy, C₁₋₃-alkyl, F₃C, HF₂C, FH₂C, H₂N— and C₁₋₃-alkoxy.

Most particularly preferred are those groups R² which are selected from among n-propyl, n-butyl, 2-propynyl, 2-butynyl, cyclohexylmethyl, cyclopentylmethyl, phenylmethyl, 2-phenylethyl, pyridylmethyl, furanylmethyl, thienylmethyl and thiazolylmethyl, wherein the above mentioned propyl, butyl, propynyl, butynyl, cyclohexylmethyl and cyclopentylmethyl groups may optionally be substituted by one or more fluorine atoms and the pyridylmethyl, furanylmethyl, thienylmethyl or thiazolylmethyl groups may optionally be substituted independently of one another by one or more groups selected from among fluorine, chlorine, bromine, methyl, F₃C, HF₂C, FH₂C— and H₂N—

Particularly preferred are those groups R² which are selected from among phenylmethyl, thienylmethyl, pyridylmethyl, particularly ortho-pyridylmethyl and thiazolylmethyl.

In the compounds of formula (I) according to the invention the group R³ is preferably selected from among hydrogen, fluorine, methyl, F₃C, HF₂C— and FH₂C, particularly preferably R³ is hydrogen.

In the compounds of formula (I) according to the invention the group R⁴ is preferably selected from among hydrogen and fluorine, particularly preferably R⁴ is hydrogen.

In a particularly preferred embodiment of the compounds according to the invention the group R³ is selected from among hydrogen, fluorine, methyl, F₃C, HF₂C— and FH₂C— and the group R⁴ is hydrogen or fluorine.

In a most particularly preferred embodiment of the compounds according to the invention the groups R³ and R⁴ are hydrogen.

In the compounds of formula (I) according to the invention the group R⁵ is preferably selected from among hydrogen, C₁₋₆-alkyl, C₂₋₆-alkenyl, C₂₋₆-alkynyl, C₃₋₇-cycloalkyl, C₃₋₇-cycloalkenyl, C₃₋₇-cycloalkenyl-C₁₋₃-alkyl, heterocyclyl, heterocyclyl-C₁₋₃-alkyl, aryl, aryl-C₁₋₃-alkyl, heteroaryl and heteroaryl-C₁₋₃-alkyl, wherein the above mentioned groups may optionally be substituted independently of one another by one or more groups selected from among fluorine, chlorine, bromine, iodine, hydroxy, carboxy, cyano, nitro, C₁₋₃-alkyl, C₁₋₃-alkoxy, C₁₋₃-alkyl-S, aryl, heteroaryl, heteroaryl-C₁₋₃-alkyl, aryl-C₁₋₃-alkyl, (R¹²)₂N—SO₂, (R¹²)₂N, (R¹²)₂N—C₁₋₃-alkyl- and (R¹²)₂N—CO—.

Particularly preferred groups R⁵ are selected from among C₁₋₆-alkyl, cyclopropyl, C₃₋₆-cycloalkyl-C₁₋₃-alkyl and phenyl-C₁₋₃-alkyl, wherein the above mentioned groups may optionally be substituted independently of one another by one or more groups selected from among fluorine, chlorine, bromine, iodine, cyano, hydroxy, carboxy, C₁₋₄-alkyl, C₁₋₄-alkoxy and (R¹²)₂N—.

Most preferably, R⁵ is a C₁₋₄-alkyl or cyclopropyl group, wherein one or more hydrogen atoms of the above mentioned groups may optionally be replaced by fluorine atoms. Of the particularly preferred C₁₋₄-alkyl groups the n-butyl group in particular is especially preferred.

In the compounds of formula (I) according to the invention the group R⁵ is preferably selected from among hydrogen, C₁₋₆-alkyl, C₂₋₆-alkenyl, C₂₋₆-alkynyl, C₃₋₇-cycloalkyl, C₃₋₇-cycloalkenyl, C₃₋₇-cycloalkenyl-C₁₋₃-alkyl, heterocyclyl, heterocyclyl-C₁₋₃-alkyl, aryl, aryl-C₁₋₃-alkyl, heteroaryl and heteroaryl-C₁₋₃-alkyl, wherein the above mentioned groups may optionally be substituted independently of one another by one or more groups selected from among fluorine, chlorine, bromine, iodine, hydroxy, carboxy, cyano, nitro, C₁₋₃-alkyl, C₁₋₃-alkoxy, C₁₋₃-alkyl-S, aryl, heteroaryl, heteroaryl-C₁₋₃-alkyl, (R¹²)₂N—SO₂, (R¹²)₂N, (R¹²)₂N—C₁₋₃-alkyl and (R¹²)₂N—CO.

Particularly preferred groups R⁵ are selected from among C₁₋₆-alkyl, cyclopropyl, C₃₋₆-cycloalkyl-C₁₋₃-alkyl and phenyl-C₁₋₃-alkyl, wherein the above mentioned groups may optionally be substituted independently of one another by one or more groups selected from among fluorine, chlorine, bromine, iodine, cyano, hydroxy, carboxy, C₁₋₄-alkyl, C₁₋₄-alkoxy- and (R¹²)₂N—.

Most particularly preferably R⁵ is a C₁₋₄-alkyl or cyclopropyl group, wherein one or more hydrogen atoms of the above mentioned groups may optionally be replaced by fluorine atoms. Of the particularly preferred C₁₋₄-alkyl groups the n-butyl group is particularly preferred.

In the compounds of formula (I) according to the invention the group R⁶ is preferably selected from among hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, C₃₋₇-cycloalkenyl, C₃₋₇-cycloalkenyl-C₁₋₃-alkyl, heterocyclyl, heterocyclyl-C₁₋₃-alkyl, aryl, aryl-C₁₋₃-alkyl, heteroaryl and heteroaryl-C₁₋₃-alkyl, wherein the above mentioned groups may optionally be substituted independently of one another by one or more groups selected from among fluorine, chlorine, bromine, iodine, hydroxy, carboxy, cyano, nitro, C₁₋₃-alkyl, C₃₋₇-cycloalkyl, heterocyclyl, heterocyclyl-C₁₋₃-alkyl, aryl, aryl-C₁₋₃-alkyl, heteroaryl, heteroaryl-C₁₋₃-alkyl, (R¹²)₂N, (R¹²)₂N—C₁₋₃-alkyl, (R¹²)₂N—CO, (R¹²)₂N—CO—N(R¹²), (R¹²)₂N—SO₂, R¹³—O— and R¹³—O—C₁₋₃-alkyl.

Particularly preferred groups R⁶ are groups selected from among hydrogen, C₁₋₆-alkyl, C₂₋₆-alkenyl, C₂₋₆-alkynyl, C₃₋₆-cycloalkyl, C₃₋₆-cycloalkyl-C₁₋₃-alkyl, heterocyclyl, heterocyclyl-C₁₋₃-alkyl, phenyl, phenyl-C₁₋₃-alkyl, heteroaryl and heteroaryl-C₁₋₃-alkyl, wherein by the above-mentioned heteroaryl groups are meant 5- or 6-membered aromatic heteroaryl groups which contain 1, 2 or 3 heteroatoms selected from among N, O and S and

wherein the above mentioned groups may optionally be substituted independently of one another by one or more groups selected from among fluorine, chlorine, bromine, carboxy, hydroxy, cyano, C₁₋₃-alkyl, C₁₋₃-alkoxy, C₁₋₃-alkoxy-C₁₋₃-alkyl, hydroxy-C₁₋₃-alkyl, C₃₋₇-cycloalkyl, heterocyclyl, heterocyclyl-C₁₋₃-alkyl, aryl, (R¹²)₂N, (R¹²)₂N—C₁₋₃-alkyl, (R¹²)₂N—CO—N(R¹²)— and (R¹²)₂N—SO₂—.

Most particularly preferred are those groups R⁶ which are selected from among hydrogen, hydrogen, C₁₋₆-alkyl, C₃₋₆-cycloalkyl, C₃₋₅-cycloalkyl-C₁₋₃-alkyl, phenyl-C₁₋₃-alkyl and tetrahydropyranyl-C₁₋₃-alkyl,

wherein the above mentioned groups may optionally be substituted independently of one another by one or more groups selected from among fluorine, pyrrolidin-1-ylmethyl, hydroxy, cyano, C₁₋₃-alkyl, C₁₋₃-alkoxy, hydroxy-C₁₋₃-alkyl, (R¹²)₂N, (R¹²)₂N—C₁₋₃-alkyl, (R¹²)₂N—CO—N(R¹²) and (R¹²)₂N—SO₂.

Particularly preferred as the group R⁶ is a cyclopropyl-C₁₋₃-alkyl or phenyl-C₁₋₃-alkyl group, wherein the phenyl group may optionally be substituted by one or more amino groups.

In the compounds of formula (I) according to the invention the group R⁷ is preferably selected from among hydrogen and a C₁₋₄-alkyl group,

wherein one or more hydrogen atoms of the C₁₋₄-alkyl group may be replaced by fluorine. Particularly preferred are those compounds wherein R⁷ denotes a hydrogen atom.

In the compounds of formula (I) according to the invention the group is R⁸ preferably selected from among heterocyclyl, heterocyclyl-C₁₋₃-alkyl, C₃₋₇-cycloalkenyl, aryl-C₁₋₃-alkyl, heteroaryl, heteroaryl-C₁₋₃-alkyl, R¹³—O, R¹³—O—C₁₋₃-alkyl, R¹⁰—SO₂—(R¹¹)N and R¹⁰—CO—(R¹¹)N, wherein the above mentioned groups may optionally be substituted independently of one another by one or more groups selected from among C₁₋₄-alkyl, fluorine, chlorine, bromine, hydroxy, oxo, carboxy, cyano, nitro, C₃₋₇-cycloalkyl, heterocyclyl, (R¹²)₂N, (R¹²)₂N—CO, R¹³—CO, R¹³—O—CO, R¹²—CO—(R¹²)N, (R¹²)₂N—CO—(R¹²)N, (R¹²)₂N—SO₂, (R¹²)₂N—SO₂—(R¹²)N, R¹²—SO₂, R¹³—O, C₁₋₄-alkyl-S, F₃C, HF₂C, FH₂C, F₃C—O, HF₂C—O, FH₂C—O— and R¹²—SO₂—(R¹²)N—.

Particularly preferred groups R⁸ are groups selected from among C₁₋₄-alkoxy, C₃₋₆-cycloalkyl-oxy, C₃₋₆-cycloalkyl-C₁₋₃-alkoxy, R¹⁰—SO₂—(R¹¹)N— and R¹⁰—CO—(R¹¹)N, wherein the above mentioned groups may optionally be substituted independently of one another by one or more groups selected from among fluorine, chlorine, bromine, carboxy, cyano, C₁₋₃-alkyl, C₁₋₃-alkoxy-, C₁₋₄-alkyl-S, R¹³—CO, R¹³—O—CO, R¹²—SO₂, F₃C, HF₂C, FH₂C, F₃C—O, HF₂C—O, FH₂C—O— and (R¹²)₂N—CO—.

In a most particularly preferred embodiment of the compounds according to the invention the group R⁸ has the meaning R¹⁰—SO₂—(R¹¹)N or R¹⁰—CO—(R¹¹)N—.

Preferred groups R⁹ are each independently selected from among hydrogen, fluorine, chlorine, bromine, methyl, F₂HC, FH₂C— and F₃C, wherein the groups hydrogen, fluorine, chlorine or bromine are particularly preferred and the group hydrogen is most preferred.

Also preferred are those compounds of formula (I) according to the invention wherein R⁸ is selected from among heterocyclyl, heterocyclyl-C₁₋₃-alkyl, C₃₋₇-cycloalkenyl, aryl-C₁₋₃-alkyl, heteroaryl, heteroaryl-C₁₋₃-alkyl, R¹³—O, R¹³—O—C₁₋₃-alkyl, R¹⁰—SO₂—(R¹¹)N— and R¹⁰—CO—(R¹¹)N, wherein the above mentioned groups may optionally be substituted independently of one another by one or more groups selected from among C₁₋₄-alkyl, fluorine, chlorine, bromine, hydroxy, oxo, carboxy, cyano, nitro, C₃₋₇-cycloalkyl, heterocyclyl, (R¹²)₂N, (R¹²)₂N—CO, R¹³—CO, R¹³—O—CO, R¹²—CO—(R¹²)N, (R¹²)₂N—CO—(R¹²)N, (R¹²)₂N—SO₂, (R¹²)₂N—SO₂—(R¹²)N, R¹²—SO₂, R¹³—O, C₁₋₄-alkyl-S, F₃C, HF₂C, FH₂C, F₃C—O, HF₂C—O, FH₂C—O— and R¹²—SO₂—(R¹²)N—, and R⁹ in each case independently of one another denotes hydrogen, fluorine, chlorine, bromine, methyl, F₂HC, FH₂C or F₃C—.

Particularly preferred are those compounds according to the invention wherein R⁸ denotes C₁₋₄-alkoxy, C₃₋₆-cycloalkyl-oxy, C₃₋₆-cycloalkyl-C₁₋₃-alkoxy, R¹⁰—SO₂—(R¹¹)N or R¹⁰—CO—(R¹¹)N, wherein the above mentioned groups may optionally be substituted independently of one another by one or more groups selected from among fluorine, chlorine, bromine, carboxy, cyano, C₁₋₃-alkyl, C₁₋₃-alkoxy, C₁₋₄-alkyl-S, R¹³—CO, R¹³—O—CO, R¹²—SO₂, —F₃C, HF₂C, FH₂C, F₃C—O, HF₂C—O, FH₂C—O— and (R¹²)₂N—CO—, and R⁹ in each case independently of one another denotes hydrogen, fluorine, chlorine or bromine.

Most particularly preferred are those compounds according to the invention wherein the group R⁸ denotes R¹⁰—SO₂—(R¹¹)N or R¹⁰—CO—(R¹¹)N— and R⁹ in each case independently of one another denotes hydrogen, fluorine, chlorine or bromine, particularly preferably hydrogen.

In the compounds of formula (I) according to the invention the group R¹⁰ is preferably selected from among C₂₋₆-alkenyl, C₂₋₆-alkynyl, C₃₋₇-cycloalkyl-C₁₋₃-alkyl, heterocyclyl, heterocyclyl-C₁₋₃-alkyl, C₃₋₇-cycloalkenyl, C₃₋₇-cycloalkenyl-C₁₋₃-alkyl, heteroaryl, heteroaryl-C₁₋₃-alkyl- and (R¹²)₂N,

wherein the above mentioned groups may optionally be substituted by one or more groups selected from among fluorine, chlorine, bromine, hydroxy, carboxy, cyano, nitro, C₁₋₃-alkyl, heterocyclyl, heterocyclyl-C₁₋₃-alkyl, C₁₋₃-alkoxy, hydroxy-C₁₋₃-alkyl, R¹²—CO(R¹²)N, R¹²—SO₂(R¹²)N, (R¹²)₂N, (R¹²)₂N—C₁₋₃-alkyl- and (R¹²)₂N—CO—.

Particularly preferred groups R¹⁰ are groups selected from among heterocyclyl, heteroaryl, heteroaryl-C₁₋₃-alkyl- and (R¹²)₂N, wherein by the above mentioned heteroaryl groups are meant 5- or 6-membered aromatic heteroaryl groups which contain 1, 2 or 3 heteroatoms selected from among N, O and S and wherein the above mentioned groups may optionally be substituted independently of one another by one or more groups selected from among fluorine, chlorine, bromine, hydroxy, cyano, C₁₋₃-alkyl, C₁₋₃-alkoxy, heterocyclyl, heterocyclyl-C₁₋₃-alkyl, hydroxy-C₁₋₃-alkyl, (R¹²)₂N— and (R¹²)₂N—C₁₋₃-alkyl.

Most particularly preferred groups R¹⁰ are groups selected from among morpholinyl, piperidinyl, 4-methylpiperidinyl, pyrrolidinyl, pyridyl and (CH₃)₂N, wherein the above mentioned groups may optionally be substituted independently of one another by one or more groups selected from among fluorine, chlorine and bromine.

In the compounds of formula (I) according to the invention the group R¹¹ is preferably selected from among C₁₋₆-alkyl, C₂₋₆-alkenyl, C₂₋₆-alkynyl, C₃₋₇-cycloalkyl-C₁₋₃-alkyl, aryl, aryl-C₁₋₃-alkyl, heterocyclyl, heterocyclyl-C₁₋₃-alkyl, heteroaryl and heteroaryl-C₁₋₃-alkyl,

wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N— and (R¹²)₂N—C₁₋₃-alkyl.

Particularly preferred groups R¹¹ are groups selected from among C₁₋₆-alkyl, heterocyclyl, heterocyclyl-C₁₋₃-alkyl, C₃₋₆-cycloalkyl-C₁₋₃-alkyl, phenyl, phenyl-C₁₋₃-alkyl, heteroaryl and heteroaryl-C₁₋₃-alkyl, while by the above-mentioned heteroaryl groups are meant 5- or 6-membered aromatic heteroaryl groups which contain 1, 2 or 3 heteroatoms selected from among N, O and S and wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N— and (R¹²)₂N—C₁₋₃-alkyl.

Most particularly preferred groups R¹¹ are groups selected from among methyl, ethyl, phenyl or 4-fluorophenyl, wherein the above mentioned groups may optionally be substituted independently of one another by one or more groups selected from among fluorine, chlorine and bromine.

Also preferred are those compounds according to the invention wherein R¹⁰ is selected from among C₂₋₆-alkenyl, C₂₋₆-alkynyl, C₃₋₇-cycloalkyl-C₁₋₃-alkyl, heterocyclyl, heterocyclyl-C₁₋₃-alkyl, C₃₋₇-cycloalkenyl, C₃₋₇-cycloalkenyl-C₁₋₃-alkyl, heteroaryl, heteroaryl-C₁₋₃-alkyl- and (R¹²)₂N, wherein the above mentioned groups may optionally be substituted by one or more groups selected from among fluorine, chlorine, bromine, hydroxy, carboxy, cyano, nitro, C₁₋₃-alkyl, heterocyclyl, heterocyclyl-C₁₋₃-alkyl, C₁₋₃-alkoxy, hydroxy-C₁₋₃-alkyl, R¹²—CO(R¹²)N, R¹²—SO₂(R¹²)N, (R¹²)₂N, (R¹²)₂N—C₁₋₃-alkyl- and (R¹²)₂N—CO—, and

R¹¹ is selected from among C₁₋₆-alkyl, C₂₋₆-alkenyl, C₂₋₆-alkynyl, C₃₋₇-cycloalkyl-C₁₋₃-alkyl, aryl, aryl-C₁₋₃-alkyl, heterocyclyl, heterocyclyl-C₁₋₃-alkyl, heteroaryl and heteroaryl-C₁₋₃-alkyl, wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N— and (R¹²)₂N—C₁₋₃-alkyl.

Also particularly preferred are compounds wherein R¹⁰ is selected from among heterocyclyl, heteroaryl, heteroaryl-C₁₋₃-alkyl- and (R¹²)₂N, wherein by the above mentioned heteroaryl groups are meant 5- or 6-membered aromatic heteroaryl groups which contain 1, 2 or 3 heteroatoms selected from among N, O and S and wherein the above mentioned groups may optionally be substituted independently of one another by one or more groups selected from among fluorine, chlorine, bromine, hydroxy, cyano, C₁₋₃-alkyl, C₁₋₃-alkoxy, heterocyclyl, heterocyclyl-C₁₋₃-alkyl, hydroxy-C₁₋₃-alkyl, (R¹²)₂N— and (R¹²)₂N—C₁₋₃-alkyl, and

R¹¹ is selected from among C₁₋₆-alkyl, heterocyclyl, heterocyclyl-C₁₋₃-alkyl, C₃₋₆-cycloalkyl-C₁₋₃-alkyl, phenyl, phenyl-C₁₋₃-alkyl, heteroaryl and heteroaryl-C₁₋₃-alkyl, while by the above-mentioned heteroaryl groups are meant 5- or 6-membered aromatic heteroaryl groups which contain 1, 2 or 3 heteroatoms selected from among N, O and S and wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N— and (R¹²)₂N—C₁₋₃-alkyl.

Also particularly preferred are compounds wherein R¹⁰ is selected from among morpholinyl, piperidinyl, 4-methylpiperidinyl, pyrrolidinyl, pyridyl and (CH₃)₂N, wherein the above mentioned groups may optionally be substituted independently of one another by one or more groups selected from among fluorine, chlorine and bromine, and

R¹¹ is selected from among methyl, ethyl, phenyl and 4-fluorophenyl, wherein the above mentioned groups may optionally be substituted independently of one another by one or more groups selected from among fluorine, chlorine and bromine.

If R¹⁰ and R¹¹ together form an alkylene bridge, a C₂₋₆-alkylene bridge is preferred, so that a heterocyclic ring is formed with the inclusion of the nitrogen atoms linked to R¹¹ and the SO₂ or CO group linked to R¹⁰, while one or two —CH₂ groups of the C₂₋₆-alkylene bridge may be replaced independently of one another by O, S or —N(R¹²)— such that in each case two O, S or N atoms or an O and an S atom are not directly connected to one another, and wherein the C atoms of the above mentioned C₂₋₆-alkylene bridge may optionally be substituted independently of one another by one or more groups selected from among fluorine, hydroxy, carboxy, F₃C, C₁₋₃-alkyl- and C₁₋₃-alkoxy.

Particularly preferred are the heterocyclic rings of formulae (IIa), (IIb), (IIc) or (IId)

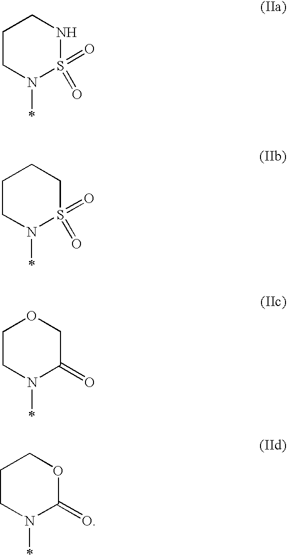

Particularly preferred are compounds of formula (I) wherein the group R⁸ combined with the groups R¹⁰ and R¹¹ forms heterocyclic rings of formulae (IIa), (IIb), (IIc) or (IId) and the other groups and radicals are defined as above or hereinafter.

In the compounds of formula (I) according to the invention the group R¹² is preferably in each case independently selected from among hydrogen and a C₁₋₆-alkyl group, wherein one or more hydrogen atoms of the C₁₋₆-alkyl group may be replaced by fluorine.

Particularly preferred groups R¹² are in each case independently of one another hydrogen or a C₁₋₆-alkyl group.

The most preferred groups R¹² are in each case independently of one another hydrogen or a methyl group.

In the compounds of formula (I) according to the invention the group R¹³ is preferably each independently selected from among hydrogen and C₁₋₃-alkyl, wherein one or more hydrogen atoms of the C₁₋₃-alkyl group may be replaced by fluorine.

Particularly preferred groups R¹³ are in each case independently of one another hydrogen or a methyl group.

Particularly preferred compounds according to the invention are listed in the following group of formulae (Ia), (Ib), (Ic), (Id) and (Ie):

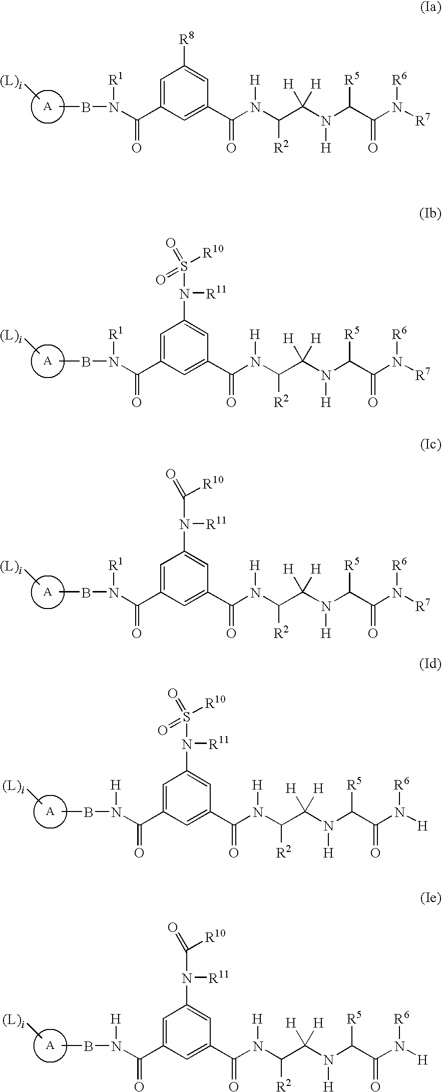

wherein
A, B, L, i, R¹, R², R⁵, R⁶, R⁷, R⁸, R¹⁰, R¹¹, R¹² and R¹³ have the meanings given above.

Particularly preferred are compounds of formula (Ia) according to the invention,

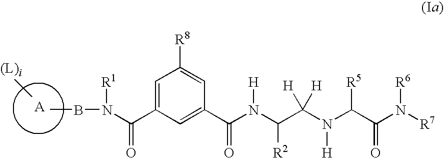

wherein
A denotes phenyl or a 5- or 6-membered aromatic heteroaryl group which contains 1, 2 or 3 heteroatoms selected from N, O and S,
L in each case independently of one another denote hydrogen, fluorine, chlorine, bromine, iodine, hydroxy, carboxy, cyano, nitro, F₃C, HF₂C, FH₂C, C₁₋₆-alkyl, C₂₋₆-alkenyl, C₂₋₆-alkynyl, C₃₋₇-cycloalkyl, C₃₋₇-cycloalkyl-C₁₋₃-alkyl, aryl, aryl-C₁₋₃-alkyl, heterocyclyl, heterocyclyl-C₁₋₃-alkyl, heteroaryl, heteroaryl-C₁₋₃-alkyl, R¹³—O, R¹³—O—C₁₋₃-alkyl, (R¹²)₂N, (R¹²)₂N—CO, R¹²—CO—(R¹²)N, (R¹²)₂N—CO—(R¹²)N, (R¹²)₂N—SO₂, R¹²—SO₂—(R¹²)N or C₁₋₃-alkyl-SO₂,
   wherein the above mentioned groups may optionally be substituted independently of one another by one or more groups selected from among fluorine, chlorine, bromine, hydroxy, oxo, carboxy, cyano, nitro, F₃C, HF₂C, FH₂C, hydroxy-C₁₋₃-alkyl, C₁₋₃-alkyl, C₁₋₃-alkoxy, (R¹²)₂N, (R¹²)₂N—C₁₋₃-alkyl- and (R¹²)₂N—CO—, and
i denotes 0, 1 or 2,
B denotes a C₁₋₄-alkylene bridge,
   wherein the C₁₋₄-alkylene bridge may optionally be substituted independently of one another by one or more groups selected from among fluorine, hydroxy, carboxy, cyano, nitro, F₃C, HF₂C, FH₂C, C₁₋₄-alkyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl, heteroaryl-C₁₋₃-alkyl, R¹³—O, (R¹²)₂N—SO₂— and (R¹²)₂N—, and
   wherein two C₁₋₄-alkyl groups bound to the same carbon atom of the C₁₋₄-alkylene bridge may be joined together forming a C₃₋₇-cycloalkyl group, and
      wherein the above mentioned groups and the C₃₋₇-cycloalkyl group formed from the C₁₋₄-alkyl groups may optionally be substituted independently of one another by one or more groups selected from among fluorine, chlorine, bromine, hydroxy, carboxy, cyano, F₃C, C₁₋₃-alkyl, C₁₋₃-alkoxy- and R¹³—O—C₁₋₃-alkyl,
R¹ denotes hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl,
   wherein the above mentioned groups may optionally be substituted independently of one another by one or more groups selected from among fluorine, chlorine, bromine, hydroxy, carboxy, cyano, nitro, F₃C, C₁₋₃-alkyl, C₁₋₃-alkoxy- and hydroxy-C₁₋₃-alkyl,
R² denotes C₁₋₆-alkyl, C₂₋₆-alkenyl, C₂₋₆-alkynyl, C₁₋₆-alkoxy-C₁₋₃-alkyl, C₁₋₆-alkyl-S—C₁₋₃-alkyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl,
   wherein the above mentioned groups may optionally be substituted independently of one another by one or more groups selected from among fluorine, chlorine, bromine, iodine, F₃C, HF₂C, FH₂C, hydroxy, carboxy, cyano, nitro, C₁₋₃-alkyl, (R¹²)₂N, (R¹²)₂N—SO₂, R¹²—CO—(R¹²)N, R¹²—SO₂(R¹²)N, (R¹²)₂N—C₁₋₃-alkyl, (R¹²)₂N—CO, R¹³—O— and R¹³—O—C₁₋₃-alkyl,
R⁵ denotes hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, C₃₋₇-cycloalkenyl, C₃₋₇-cycloalkenyl-C₁₋₃-alkyl, heterocyclyl, heterocyclyl-C₁₋₃-alkyl, aryl, aryl-C₁₋₃-alkyl, heteroaryl, or heteroaryl-C₁₋₃-alkyl,
   wherein the above mentioned groups may optionally be substituted independently of one another by one or more groups selected from among fluorine, chlorine, bromine, iodine, hydroxy, carboxy, cyano, nitro, C₁₋₃-alkyl, C₁₋₃-alkoxy, C₁₋₃-alkyl-S, aryl, heteroaryl, heteroaryl-C₁₋₃-alkyl, aryl-C₁₋₃-alkyl, (R¹²)₂N—SO₂, (R¹²)₂N, (R¹²)₂N—C₁₋₃-alkyl- and (R¹²)₂N—CO—,
R⁶ denotes hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, C₃₋₇-cycloalkenyl, C₃₋₇-cycloalkenyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl,
   wherein the above mentioned groups may optionally be substituted independently of one another by one or more groups selected from among fluorine, chlorine, bromine, iodine, hydroxy, carboxy, cyano, nitro, C₁₋₃-alkyl, C₃₋₇-cycloalkyl, heterocyclyl, heterocyclyl-C₁₋₃-alkyl, aryl, aryl-C₁₋₃-alkyl, heteroaryl, heteroaryl-C₁₋₃-alkyl, (R¹²)₂N, (R¹²)₂N—C₁₋₃-alkyl, (R¹²)₂N—CO, (R¹²)₂N—CO—N(R¹²), (R¹²)₂N—SO₂, R¹³—O— and R¹³—O—C₁₋₃-alkyl,
R⁷ denotes hydrogen or C₁₋₄-alkyl,
   wherein one or more hydrogen atoms of the C₁₋₄-alkyl group may be replaced by fluorine,
R⁸ denotes heterocyclyl, heterocyclyl-C₁₋₃-alkyl, C₃₋₇-cycloalkenyl, aryl-C₁₋₃-alkyl, heteroaryl, heteroaryl-C₁₋₃-alkyl, R¹³—O, R¹³—O—C₁₋₃-alkyl, R¹⁰—SO₂—(R¹¹)N or R¹⁰—CO—(R¹¹)N,
   wherein the above mentioned groups may optionally be substituted independently of one another by one or more groups selected from among C₁₋₄-alkyl, fluorine, chlorine, bromine, hydroxy, oxo, carboxy, cyano, nitro, C₃₋₇-cycloalkyl, heterocyclyl, (R¹²)₂N, (R¹²)₂N—CO, R¹³—CO, R¹³—O—CO, R¹²—CO—(R¹²)N, (R¹²)₂N—CO—(R¹²)N, (R¹²)₂N—SO₂, (R¹²)₂N—SO₂—(R¹²)N, R¹²—SO₂, R¹³—O, C₁₋₄-alkyl-S, F₃C, HF₂C, FH₂C, F₃C—O, HF₂C—O, FH₂C—O— and R¹²—SO₂—(R¹²)N—,
R¹⁰ denotes C₂₋₆-alkenyl, C₂₋₆-alkynyl, C₃₋₇-cycloalkyl-C₁₋₃-alkyl, heterocyclyl, heterocyclyl-C₁₋₃-alkyl, C₃₋₇-cycloalkenyl, C₃₋₇-cycloalkenyl-C₁₋₃-alkyl, heteroaryl, heteroaryl-C₁₋₃-alkyl or (R¹²)₂N,
   wherein the above mentioned groups may optionally be substituted by one or more groups selected from among fluorine, chlorine, bromine, hydroxy, carboxy, cyano, nitro, C₁₋₃-alkyl, heterocyclyl, heterocyclyl-C₁₋₃-alkyl, C₁₋₃-alkoxy, hydroxy-C₁₋₃-alkyl, R¹²—CO(R¹²)N, R¹²—SO₂(R¹²)N, (R¹²)₂N, (R¹²)₂N—C₁₋₃-alkyl- and (R¹²)₂N—CO—, and
R¹¹ denotes C₁₋₆-alkyl, C₂₋₆-alkenyl, C₂₋₆-alkynyl, C₃₋₇-cycloalkyl-C₁₋₃-alkyl, aryl, aryl-C₁₋₃-alkyl, heterocyclyl, heterocyclyl-C₁₋₃-alkyl, heteroaryl or heteroaryl-C₁₋₃-alkyl,
   wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N and (R¹²)₂N—C₁₋₃-alkyl, or
R¹⁰ and R¹¹ together form a C₂₋₆-alkylene bridge, so that a heterocyclic ring is formed with the inclusion of the nitrogen atom linked to R¹¹ and the SO₂— or CO— group linked to R¹⁰,
   wherein one or two —CH₂ groups of the C₂₋₆-alkylene bridge may be replaced independently of one another by O, S or —N(R¹²)— such that in each case two O, S or N atoms or an O and an S atom are not directly connected to one another, and
   wherein the C atoms of the above mentioned C₂₋₆-alkylene bridge may optionally be substituted independently of one another by one or more groups selected from among fluorine, hydroxy, carboxy, F₃C, C₁₋₃-alkyl and C₁₋₃-alkoxy,
R¹² denotes hydrogen or a C₁₋₆-alkyl group
   wherein one or more hydrogen atoms of the C₁₋₆-alkyl group may be replaced by fluorine,
R¹³ denotes hydrogen or a C₁₋₃-alkyl group
   wherein one or more hydrogen atoms of the C₁₋₃-alkyl group may be replaced by fluorine.

Also particularly preferred are those compounds of formulae (Ib) and (Ic) according to the invention

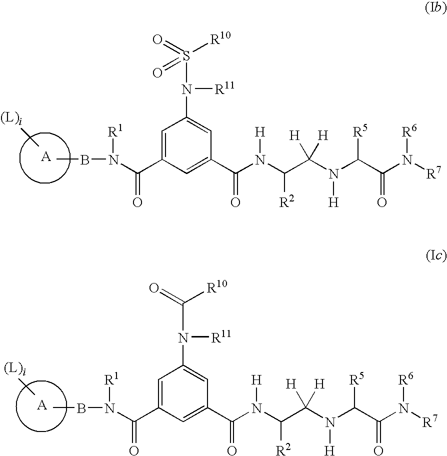

wherein
A denotes phenyl, thienyl, thiazolyl, pyrazolyl or pyridyl,
L in each case independently of one another denote hydrogen, fluorine, chlorine, bromine, cyano, hydroxy, C₁₋₆-alkyl, C₁₋₆-alkoxy, C₃₋₇-cycloalkyl, C₃₋₇-cycloalkyl-C₁₋₃-alkyl, phenyl, (R¹²)₂N, (R¹²)₂N—CO, R¹²—CO—(R¹²)N, (R¹²)₂N—CO—(R¹²)N, R¹²—SO₂—(R¹²)N or (R¹²)₂N—SO₂,
   wherein the above mentioned groups may optionally be substituted by one or more fluorine atoms,
i denotes 0, 1 or 2,
B denotes a C₁₋₄-alkylene bridge,
   wherein the C₁₋₄-alkylene bridge may optionally be substituted independently of one another by one or more groups selected from among fluorine, C₁₋₄-alkyl, phenyl or benzyl, and
   wherein two C₁₋₄-alkyl groups bound to the same carbon atom of the C₁₋₄-alkylene bridge may be joined together, forming a C₃₋₆-cycloalkyl group, and
   wherein the above mentioned groups and the C₃₋₆-cycloalkyl group formed from the C₁₋₄-alkyl groups may optionally be substituted independently of one another by one or more groups selected from among fluorine, hydroxy and C₁₋₃-alkoxy,
R¹ denotes hydrogen, C₁₋₄-alkyl, C₃₋₄-alkenyl, C₃₋₆-cycloalkyl, C₃₋₆-cycloalkyl-C₁₋₃-alkyl,
   wherein the above mentioned groups may optionally be substituted independently of one another by one or more groups selected from among fluorine, hydroxy and C₁₋₃-alkoxy,
R² denotes C₁₋₆-alkyl, C₂₋₆-alkynyl, C₃₋₆-cycloalkyl-C₁₋₃-alkyl, heterocyclyl-C₁₋₃-alkyl, phenyl, phenyl-C₁₋₃-alkyl, heteroaryl or heteroaryl-C₁₋₃-alkyl,
   wherein by the above mentioned heteroaryl groups are meant 5- or 6-membered aromatic heteroaryl groups which contain 1, 2 or 3 heteroatoms selected from among N, O and S and
   wherein the above mentioned groups may optionally be substituted independently of one another by one or more groups selected from among fluorine, chlorine, bromine, iodine, cyano, hydroxy, C₁₋₃-alkyl-, F₃C, HF₂C, FH₂C, H₂N— and C₁₋₃-alkoxy,
R⁵ denotes C₁₋₆-alkyl, cyclopropyl, C₃₋₆-cycloalkyl-C₁₋₃-alkyl or phenyl-C₁₋₃-alkyl,
   wherein the above mentioned groups may optionally be substituted independently of one another by one or more groups selected from among fluorine, chlorine, bromine, iodine, cyano, hydroxy, carboxy, C₁₋₄-alkyl, C₁₋₄-alkoxy- and (R¹²)₂N—,
R⁶ denotes hydrogen, C₁₋₆-alkyl, C₂₋₆-alkenyl, C₂₋₆-alkynyl, C₃₋₆-cycloalkyl, C₃₋₆-cycloalkyl-C₁₋₃-alkyl, heterocyclyl, heterocyclyl-C₁₋₃-alkyl, phenyl, phenyl-C₁₋₃-alkyl, heteroaryl or heteroaryl-C₁₋₃-alkyl,
   wherein by the above-mentioned heteroaryl groups are meant 5- or 6-membered aromatic heteroaryl groups which contain 1, 2 or 3 heteroatoms selected from among N, O and S and
   wherein the above mentioned groups may optionally be substituted independently of one another by one or more groups selected from among fluorine, chlorine, bromine, carboxy, hydroxy, cyano, C₁₋₃-alkyl, C₁₋₃-alkoxy, C₁₋₃-alkoxy-C₁₋₃-alkyl, hydroxy-C₁₋₃-alkyl, C₃₋₇-cycloalkyl, heterocyclyl, heterocyclyl-C₁₋₃-alkyl, aryl, (R¹²)₂N, (R¹²)₂N—C₁₋₃-alkyl, (R¹²)₂N—CO—N(R¹²)— and (R¹²)₂N—SO₂—,
R⁷ denotes hydrogen or C₁₋₄-alkyl,
   wherein one or more hydrogen atoms of the C₁₋₄-alkyl group may be replaced by fluorine,
R¹⁰ denotes heterocyclyl, heteroaryl, heteroaryl-C₁₋₃-alkyl or (R¹²)₂N,
   wherein by the above mentioned heteroaryl groups are meant 5- or 6-membered aromatic heteroaryl groups which contain 1, 2 or 3 heteroatoms selected from among N, O and S and
   wherein the above mentioned groups may optionally be substituted independently of one another by one or more groups selected from among fluorine, chlorine, bromine, hydroxy, cyano, C₁₋₃-alkyl, C₁₋₃-alkoxy, heterocyclyl, heterocyclyl-C₁₋₃-alkyl, hydroxy-C₁₋₃-alkyl, (R¹²)₂N— and (R¹²)₂N—C₁₋₃-alkyl, and
R¹¹ denotes C₁₋₆-alkyl, heterocyclyl, heterocyclyl-C₁₋₃-alkyl, C₃₋₆-cycloalkyl-C₁₋₃-alkyl, phenyl, phenyl-C₁₋₃-alkyl, heteroaryl or heteroaryl-C₁₋₃-alkyl, while by the above-mentioned heteroaryl groups are meant 5- or 6-membered aromatic heteroaryl groups which contain 1, 2 or 3 heteroatoms selected from among N, O and S and
   wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N and (R¹²)₂N—C₁₋₃-alkyl, or
R¹⁰ and R¹¹ together form a C₂₋₆-alkylene bridge, so that a heterocyclic ring is formed with the inclusion of the nitrogen atom linked to R¹¹ and the SO₂— or CO— group linked to R¹⁰,
   wherein one or two —CH₂ groups of the C₂₋₆-alkylene bridge may be replaced independently of one another by O, S or —N(R¹²)— such that in each case two O, S or N atoms or an O and an S atom are not directly connected to one another, and
   wherein the C atoms of the above mentioned C₂₋₆-alkylene bridge may optionally be substituted independently of one another by one or more groups selected from among fluorine, hydroxy, carboxy, F₃C, C₁₋₃-alkyl and C₁₋₃-alkoxy, and
R¹² denotes hydrogen or a C₁₋₆-alkyl group
   wherein one or more hydrogen atoms of the C₁₋₆-alkyl group may be replaced by fluorine.

Also particularly preferred are those compounds of formulae (Id) and (Ie) according to the invention

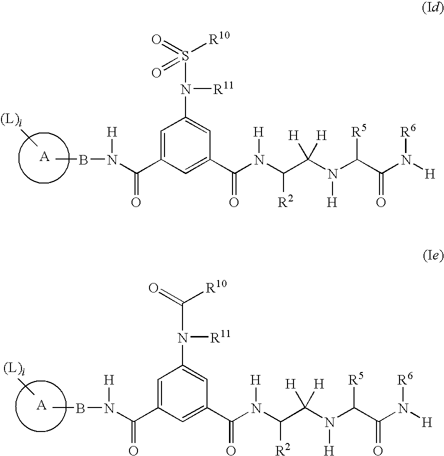

wherein
A denotes phenyl, thienyl, thiazolyl, pyrazolyl or pyridyl,
L in each case independently of one another denotes hydrogen, fluorine, chlorine, bromine, hydroxy, C₁₋₄-alkyl or C₁₋₄-alkoxy,
   wherein the above mentioned groups may optionally be substituted by one or more fluorine atoms, and
i denotes 0, 1 or 2, preferably 0 or 1
B is selected from among

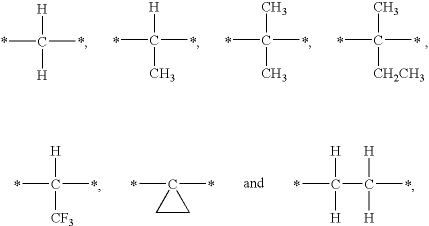

wherein one or more hydrogen atoms may optionally be replaced by fluorine.
R² denotes n-propyl, n-butyl, 2-propynyl, 2-butynyl, cyclohexylmethyl, cyclopentylmethyl, phenylmethyl, 2-phenylethyl, pyridylmethyl, furanylmethyl, thienylmethyl or thiazolylmethyl,
   wherein the above mentioned propyl, butyl, propynyl, butynyl, cyclohexylmethyl and cyclopentylmethyl groups may optionally be substituted by one or more fluorine atoms and the pyridylmethyl, furanylmethyl, thienylmethyl or thiazolylmethyl groups may optionally be substituted independently of one another by one or more groups selected from among fluorine, chlorine, bromine, methyl, F₃C, HF₂C, FH₂C— and H₂N,
R⁵ denotes C₁₋₄-alkyl or cyclopropyl,
   wherein one or more hydrogen atoms of the above mentioned groups may optionally be replaced by fluorine atoms,
R⁶ denotes hydrogen, C₁₋₆-alkyl, C₃₋₆-cycloalkyl, C₃₋₅-cycloalkyl-C₁₋₃-alkyl, phenyl-C₁₋₃-alkyl or tetrahydropyranyl-C₁₋₃-alkyl,
   wherein the above mentioned groups may optionally be substituted independently of one another by one or more groups selected from among fluorine, pyrrolidin-1-ylmethyl, hydroxy, cyano, C₁₋₃-alkyl, C₁₋₃-alkoxy, hydroxy-C₁₋₃-alkyl, H₂N, H₂N—C₁₋₃-alkyl, H₂N—CO—NH— and H₂N—SO₂—,
R¹⁰ denotes morpholinyl, piperidinyl, 4-methylpiperidinyl, pyrrolidinyl, pyridyl- and (CH₃)₂N,
   wherein the above mentioned groups may optionally be substituted independently of one another by one or more groups selected from among fluorine, chlorine and bromine,
R¹¹ denotes methyl, ethyl, phenyl or 4-fluorophenyl,
   wherein the above mentioned groups may optionally be substituted independently of one another by one or more groups selected from among fluorine, chlorine and bromine, or
R¹⁰ and R¹¹ with the inclusion of the nitrogen atom bound to R¹¹ and the SO₂-group bound to R¹⁰, together form a heterocyclic ring of formulae (IIa), (IIb), (IIc) or (IId)

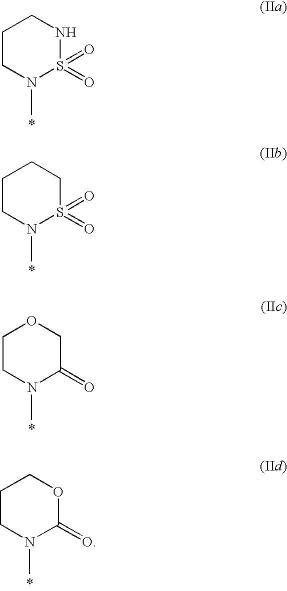

Particularly preferred individual compounds are selected from among

| | | Example |
|---|---|---|
| Compound | | No: |
| (1) | 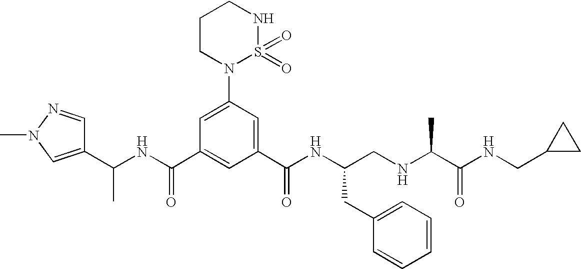 | 1 |
| (2) | 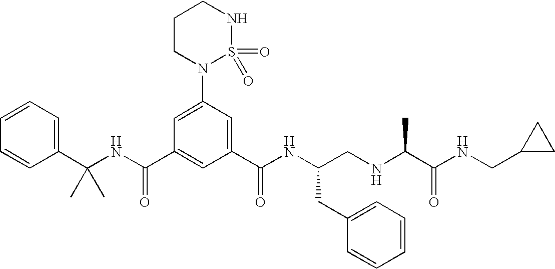 | 1.1 |
| (3) | 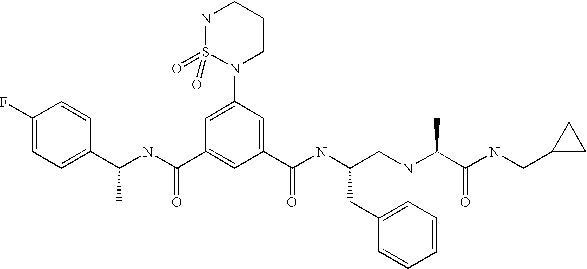 | 1.2 |
| (4) | 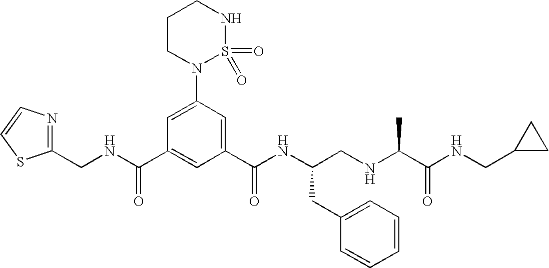 | 1.3 |
| (5) | 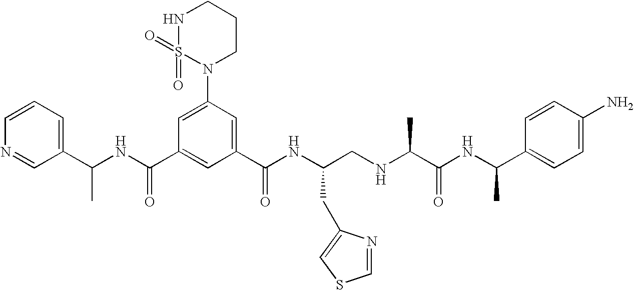 | 1.4 |
| (6) | 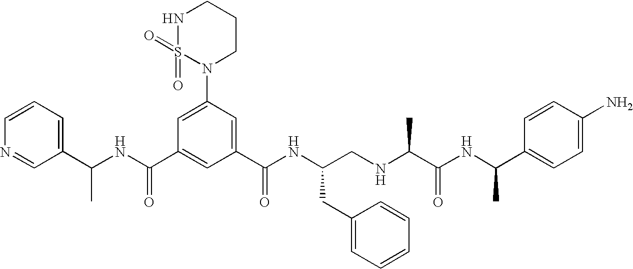 | 1.5 |
| (7) | 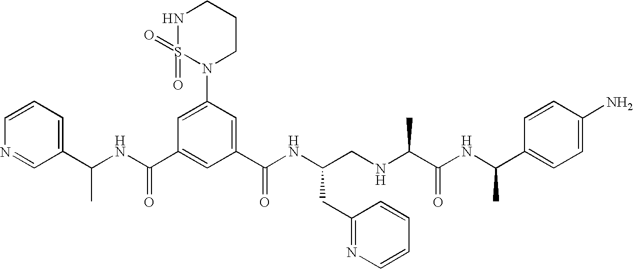 | 1.6 |
| (8) | 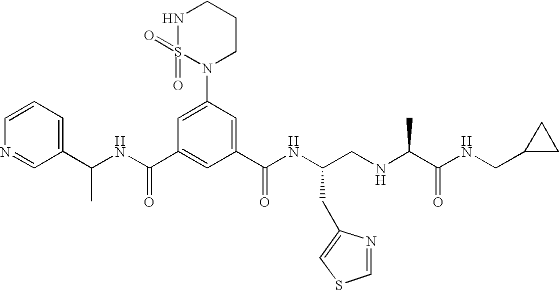 | 1.7 |
| (9) | 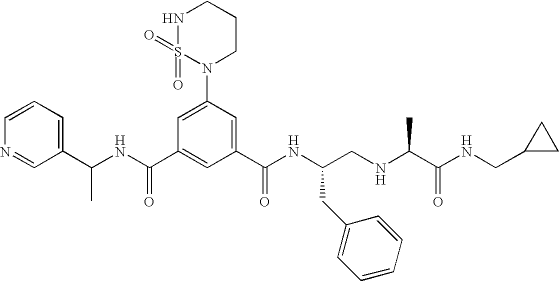 | 1.8 |
| (10) | 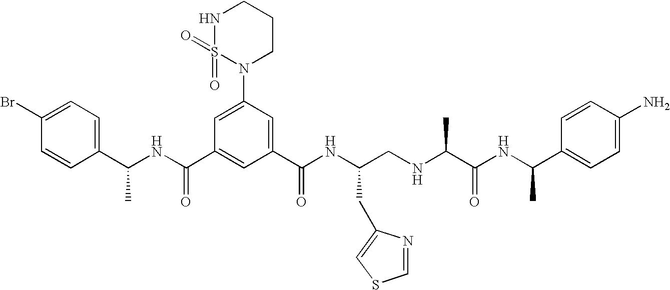 | 1.9 |
| (11) | 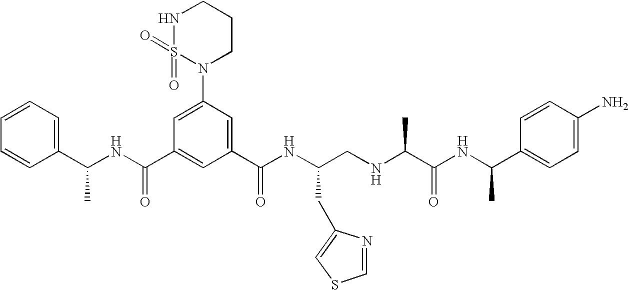 | 1.10 |
| (12) | 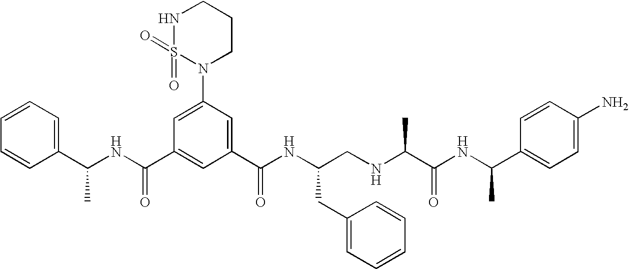 | 1.11 |
| (13) | 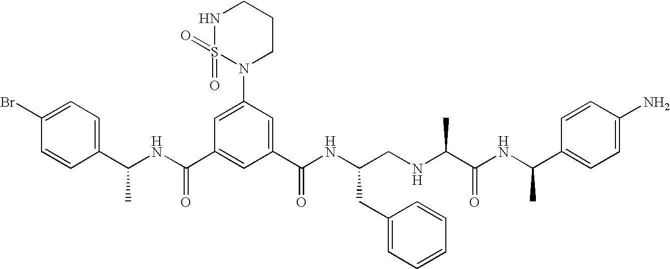 | 1.12 |
| (14) | 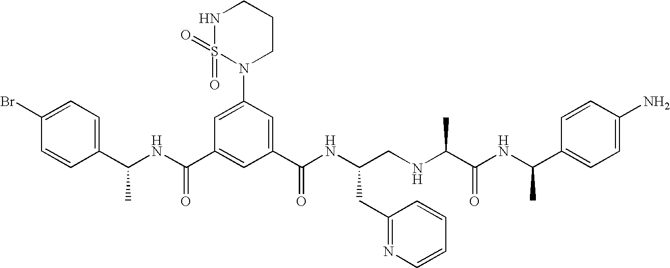 | 1.13 |
| (15) | 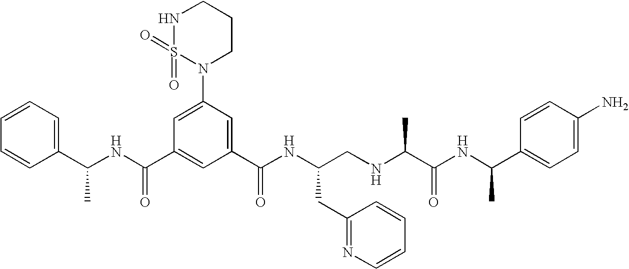 | 1.14 |
| (16) | 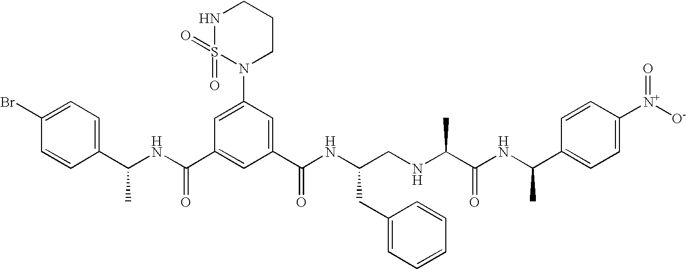 | 1.15 |
| (17) | 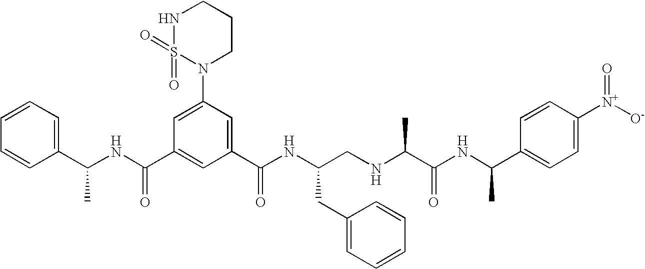 | 1.16 |
| (18) | 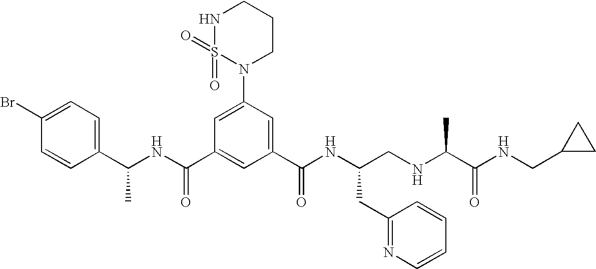 | 1.17 |
| (19) | 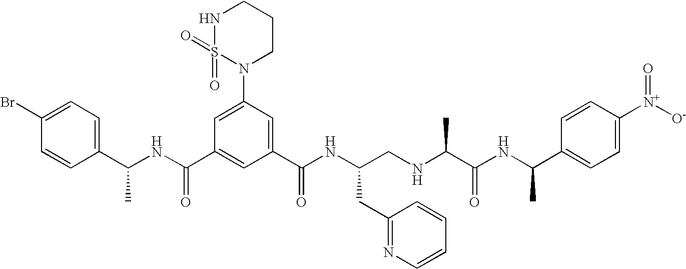 | 1.18 |
| (20) | 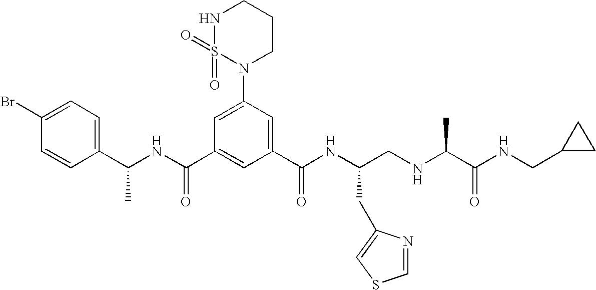 | 1.19 |
| (21) | 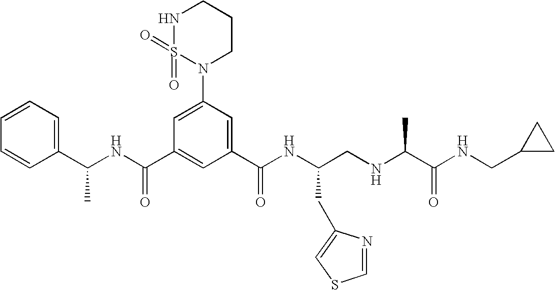 | 1.20 |
| (22) | 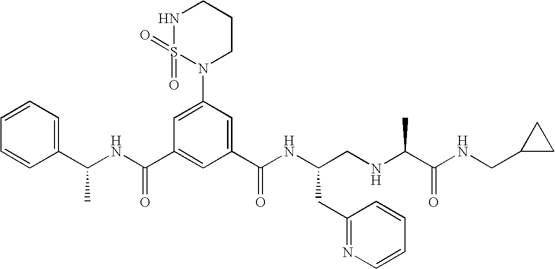 | 1.21 |
| (23) | 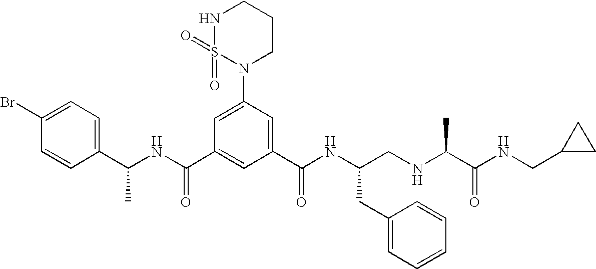 | 1.22 |
| (24) | 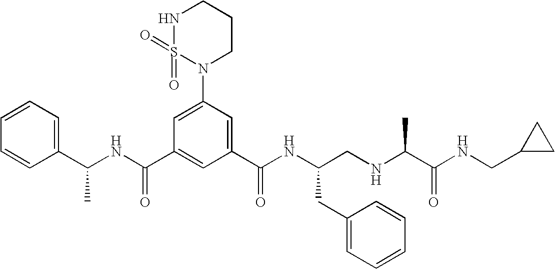 | 1.23 |
| (25) | 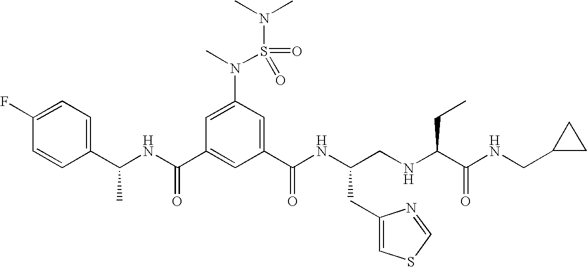 | 2 |
| (26) | 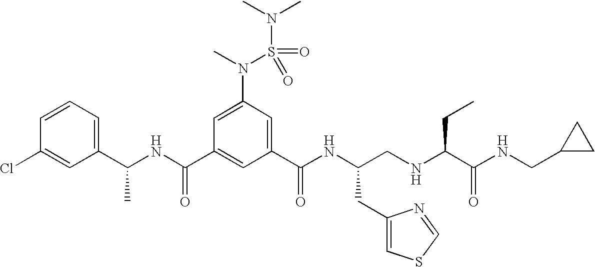 | 2.1 |
| (27) | 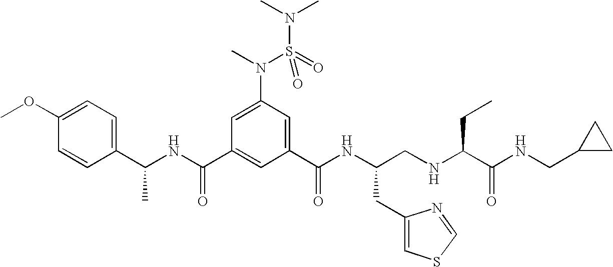 | 2.2 |
| (28) | 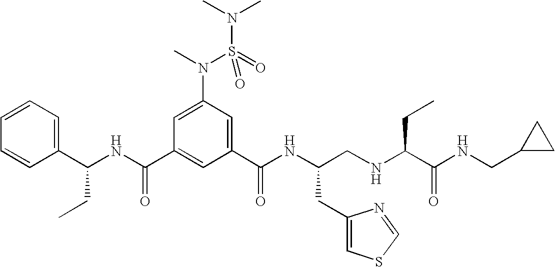 | 2.3 |
| (29) | 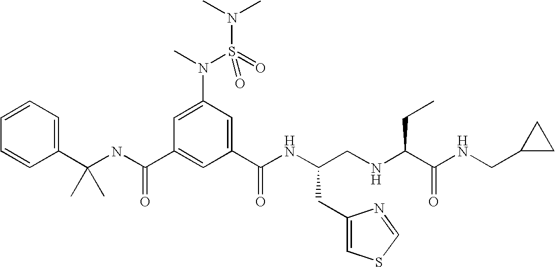 | 2.4 |
| (30) | 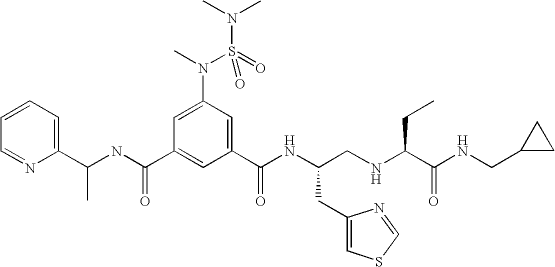 | 2.5 |
| (31) | 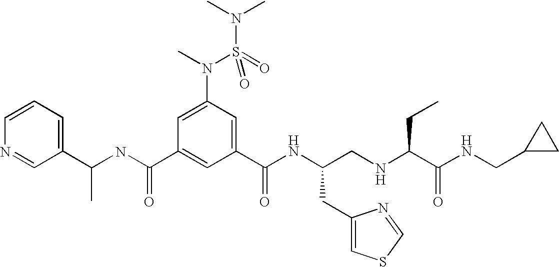 | 2.6 |
| (32) | 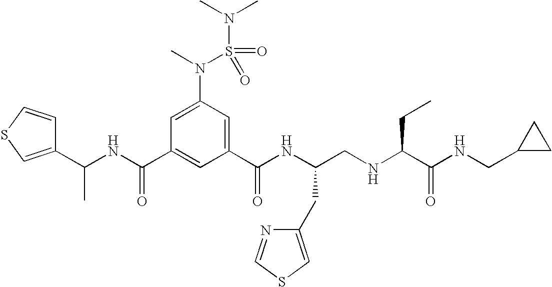 | 2.7 |
| (33) | 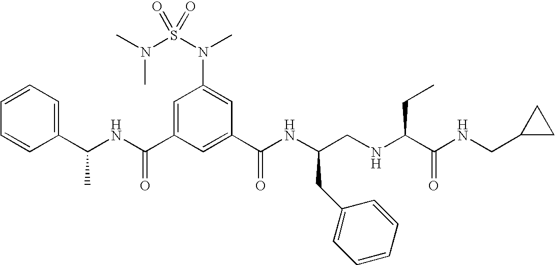 | 2.8 |
| (34) | 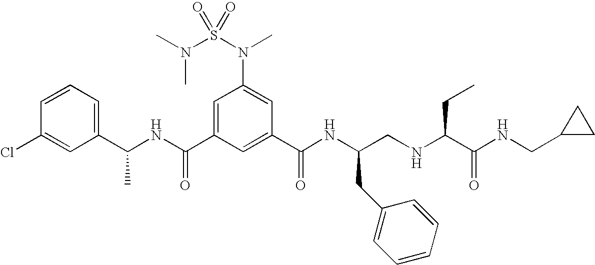 | 2.9 |
| (35) | 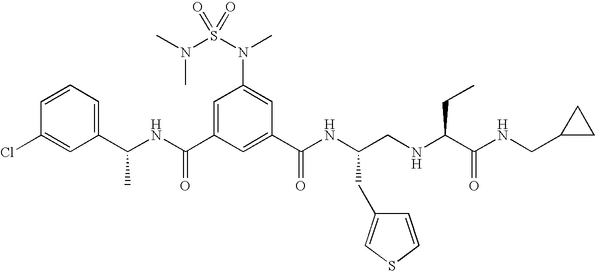 | 2.10 |
| (36) | 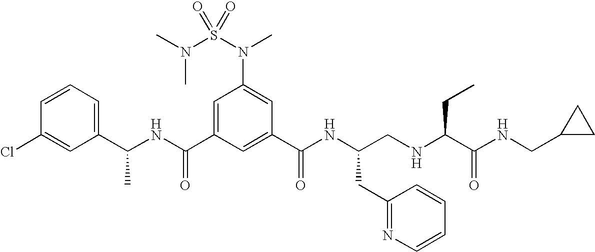 | 2.11 |
| (37) | 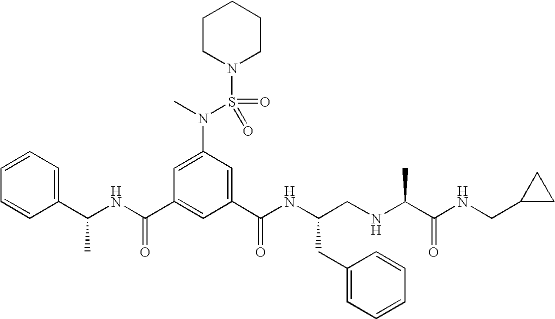 | 3 |
| (38) | 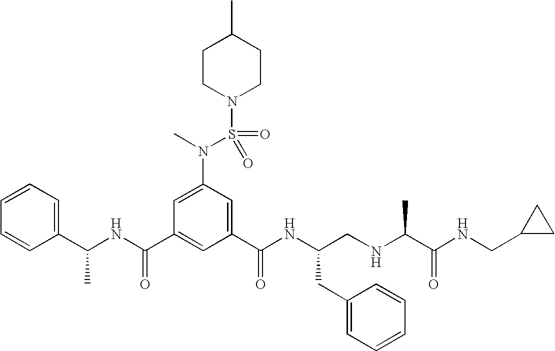 | 3.1 |
| (39) | 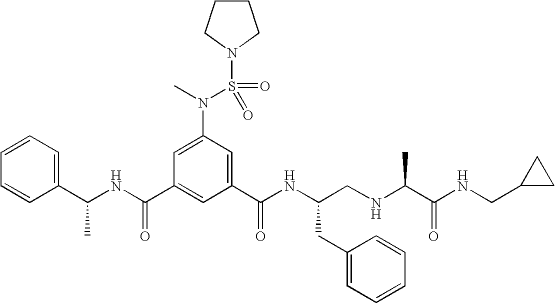 | 3.2 |
| (40) | 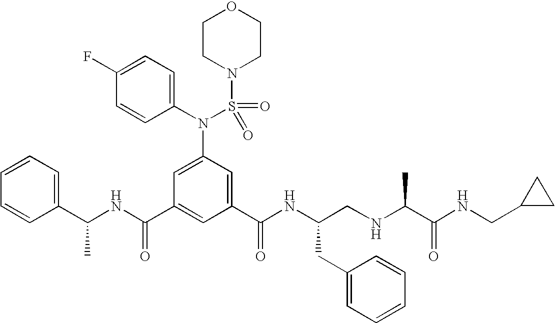 | 4 |
| (41) | 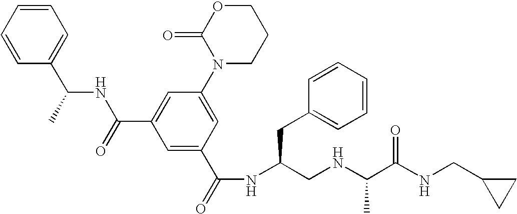 | 5 |
| (42) | 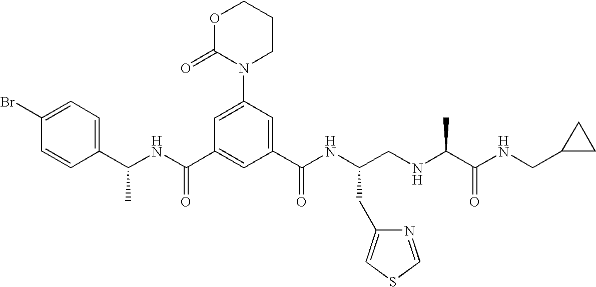 | 5.1 |
| (43) | 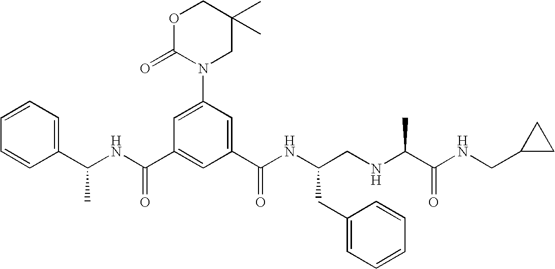 | 5.2 |
| (44) | 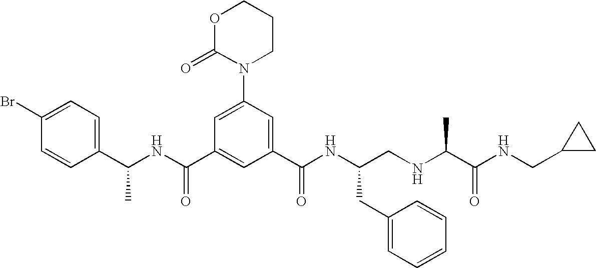 | 5.3 |
| (45) | 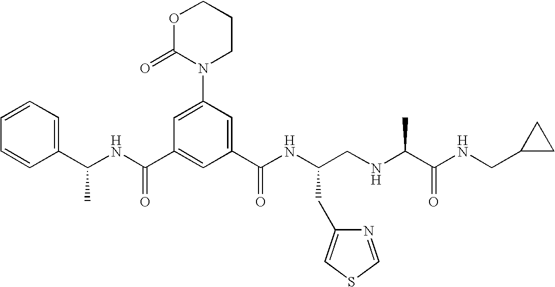 | 5.4 |
| (46) | 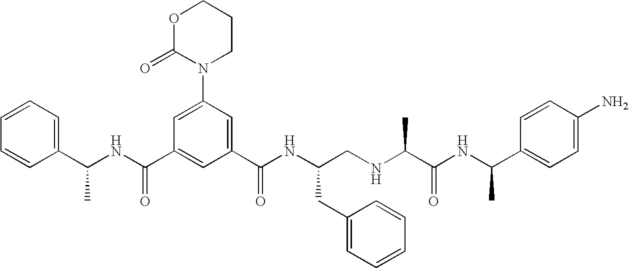 | 5.5 |

Some terms used hereinbefore and hereinafter to describe the compounds according to the invention are defined below.

The term halogen denotes an atom selected from among F, Cl, Br and I.

The term C₁₋ₙ-alkyl, wherein n may have a value of from 1 to 10, unless otherwise stated, denotes a saturated, branched or unbranched hydrocarbon group with 1 to n C atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl etc.

The term C₁₋ₙ-alkylene, wherein n may have a value of from 1 to 8, unless otherwise stated, denotes a saturated, branched or unbranched hydrocarbon bridge with 1 to n C atoms. Examples of such groups include methylene (—CH₂—), ethylene (—CH₂—CH₂—), 1-methyl-methylene (—CH(CH₃)—). 1-methyl-ethylene (—CH(CH₃)—CH₂—), 1,1-dimethyl-ethylene (—C(CH₃)₂—CH₂—), n-prop-1,3-ylene (—CH₂—CH₂—CH₂—), 1-methylprop-1,3-ylene (—CH(CH₃)—CH₂—CH₂—), 2-methylprop-1,3-ylene (—CH₂—CH(CH₃)—CH₂—), etc., as well as the corresponding mirror-symmetrical forms.

The term C₂₋ₙ-alkenyl, wherein n may have a value of from 2 to 6, unless otherwise stated, denotes a branched or unbranched hydrocarbon group with 2 to n C atoms and a C═C-double bond. Examples of such groups include ethenyl, 1-propenyl, 2-propenyl, iso-propenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 4-hexenyl, 5-hexenyl etc.

The term C₂₋ₙ-alkynyl, wherein n may have a value of from 2 to 6, unless otherwise stated, denotes a branched or unbranched hydrocarbon group with 2 to n C atoms and a C≡C-triple bond. Examples of such groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl etc.

The term C₁₋ₙ-alkoxy or C₁₋ₙ-alkyloxy denotes a C₁₋ₙ-alkyl-O group, wherein C₁₋ₙ-alkyl is as hereinbefore defined. Examples of such groups include methoxy, ethoxy, n-propoxy, iso-propoxy, n-butoxy, iso-butoxy, sec-butoxy, tert-butoxy, n-pentoxy, iso-pentoxy, neo-pentoxy, tert-pentoxy, n-hexoxy, iso-hexoxy etc.

The term C₃₋ₙ-cycloalkyl denotes a saturated monocyclic group with 3 to n C atoms. Examples of such groups include cyclopropyl, cyclobutyl, cyclopentyl, cyclohexyl, cycloheptyl.

The term C₃₋ₙ-cycloalkyloxy denotes a C₃₋ₙ-cycloalkyl-O group, wherein C₃₋ₙ-cycloalkyl is as hereinbefore defined. Examples of such groups include cyclopropyloxy, cyclobutyloxy, cyclopentyloxy, cyclohexyloxy, cycloheptyloxy etc.

The term C₃₋ₙ-cycloalkyl-C₁₋ₙ-alkoxy denotes a C₃₋ₙ-cycloalkyl group, wherein C₃₋ₙ-cycloalkyl is as hereinbefore defined and which is linked to a C₁₋ₙ-alkoxy group through a carbon atom of the C₁₋ₙ-alkoxy group. Examples of such groups include cyclopropylmethyloxy, cyclobutylethyloxy, cyclopentylmethyloxy, cyclohexylmethyloxy, cyclohexylethyloxy etc.

The term C₃₋ₙ-cycloalkenyl denotes a C₃₋ₙ-cycloalkyl group which is as hereinbefore defined and additionally has at least one C═C-double bond, but is not of an aromatic nature.

The term heterocyclyl used in this application denotes a saturated five-, six- or seven-membered ring system or a 5-12 membered bicyclic ring system which includes one, two, three or four heteroatoms, selected from N, O and/or S, such as for example a morpholinyl, piperidinyl, piperazinyl, thiomorpholinyl, oxathianyl, dithianyl, dioxanyl, pyrrolidinyl, tetrahydrofuranyl, dioxolanyl, oxathiolanyl, imidazolidinyl, tetrahydropyranyl, pyrrolinyl, tetrahydrothienyl, oxazolidinyl, homopiperazinyl, homopiperidinyl, homomorpholinyl, homothiomorpholinyl, azetidinyl, 1,3-diazacyclohexanyl or pyrazolidinyl group.

The term aryl used in this application denotes a phenyl, biphenyl, indanyl, indenyl, 6,7,8,9-tetrahydrobenzocycloheptenyl, 1,2,3,4-tetrahydronaphthyl or naphthyl group.

The term heteroaryl used in this application denotes a heterocyclic, mono- or bicyclic aromatic ring system which comprises in addition to at least one C atom one or more heteroatoms selected from N, O and/or S, while the term heteroaryl also includes the partially hydrogenated heterocyclic, aromatic ring systems. Examples of such groups are pyrrolyl, furanyl, thienyl, pyridyl-N-oxide, thiazolyl, imidazolyl, oxazolyl, triazinyl, triazolyl, triazolyl, 1,2,4-oxadiazoyl, 1,3,4-oxadiazoyl, 1,2,5-oxadiazoyl, isothiazolyl, isoxazolyl, 1,2,4-thiadiazolyl, 1,3,4-thiadiazolyl, 1,2,5-thiadiazolyl, pyrazolyl, pyrimidyl, pyridazinyl, pyrazinyl, tetrazolyl, pyridyl, indolyl, isoindoyl, indolizinyl, imidazopyridinyl, imidazo[1,2-a]pyridinyl, pyrrolopyrimidinyl, purinyl, pyridopyrimidinyl, pteridinyl, pyrimidopyrimidinyl, benzofuranyl, benzothienyl, quinolinyl, isoquinolinyl, quinazolinyl, quinoxalinyl, cinnolinyl, phthalazinyl, isobenzofuranyl, isobenzothienyl, thieno[3,2-b]thiophenyl, thieno[3,2-b]pyrrolyl, thieno[2,3-d]imidazolyl, naphthyridinyl, indazolyl, pyrrolopyridinyl, oxazolopyridinyl, benzimidazolyl, benzoxazolyl, benzisoxazolyl, benzothiazolyl, benzoisothiazolyl, benzoxadiazolyl, benzothiadiazolyl, 1,3-benzodioxolyl, 2,3-dihydrobenzofuranyl, 1,3-dihydroisobenzofuranyl, 2,3-dihydrobenzo[1,4]dioxinyl, 3,4-dihydrobenzo[1,4]oxazinyl, benzo[1,4]-oxazinyl, 2,3-dihydroindolyl, 2,3-dihydroisoindolyl, 1,2,3,4-tetrahydroquinolinyl, 1,2,3,4-tetrahydroisoquinolinyl, 2-oxo-2,3-dihydrobenzoimidazolyl, 2-oxo-2,3-dihydroindolyl, pyrazolo[1,5-a]pyridinyl, pyrazolo[1,5-a]pyrimidinyl, chromanyl, chromenyl, chromonyl, isochromenyl, isochromanyl, dihydroquinolin-4-onyl, dihydroquinolin-2-onyl, quinolin-4-onyl, isoquinolin-2-onyl, imidazo[1,2-a]pyrazinyl, 1-oxoindanyl, benzoxazol-2-onyl, imidazo[4,5-d]thiazolyl or 6,7-dihydropyrrolizinyl groups.

Preferred heteroaryl groups are furanyl, thienyl, thiazolyl, imidazolyl-isoxazolyl, pyrazolyl, pyridyl, indolyl, benzofuranyl-1,3-benzodioxolyl, 2,3-dihydrobenzofuranyl and 2,3-dihydrobenzo[1,4]dioxinyl.

The definition pyrazole includes the isomers 1H-, 3H- and 4H-pyrazole. Preferably pyrazolyl denotes 1H-pyrazolyl.

The definition imidazole includes the isomers 1H-, 2H- and 4H-imidazole. A preferred definition of imidazolyl is 1H-imidazolyl.

The definition triazole includes the isomers 1H-, 3H- and 4H-[1,2,4]-triazole as well as 1H-, 2H- and 4H-[1,2,3]-triazole. The definition triazolyl therefore includes 1H-[1,2,4]-triazol-1,3- and -5-yl, 3H-[1,2,4]-triazol-3- and -5-yl, 4H-[1,2,4]-triazol-3,4- and -5-yl, 1H-[1,2,3]-triazol-1,4- and -5-yl, 2H-[1,2,3]-triazol-2,4- and -5-yl as well as 4H-[1,2,3]-triazol-4- and -5-yl.

The term tetrazole includes the isomers 1H-, 2H- and 5H-tetrazole. The definition tetrazolyl therefore includes 1H-tetrazol-1- and -5-yl, 2H-tetrazol-2- and -5-yl as well as 5H-tetrazol-5-yl.

The definition indole includes the isomers 1H- and 3H-indole. The term indolyl preferably denotes 1H-indol-1-yl.

The definition isoindole includes the isomers 1H- and 2H-isoindole.

Generally, the bonding to one of the above-mentioned heterocyclic or heteroaromatic groups may take place via a C atom or optionally an N atom.

Within the scope of this application, in the definition of possible substituents, these may also be represented in the form of a structural formula. An asterisk (*) in the structural formula of the substituent indicates the point of connection to the remainder of the molecule. Thus, for example, the groups N-piperidinyl (a), 4-piperidinyl (b), 2-tolyl (c), 3-tolyl (d) and 4-tolyl (e) are shown as follows:

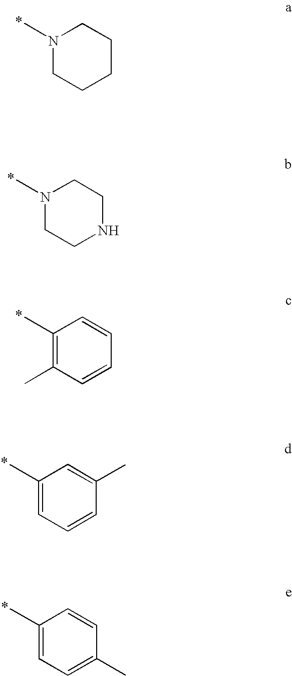

If there is no asterisk (*) in the structural formula of the substituent, every hydrogen atom may be removed from the substituent and the valency thus freed may be used as a binding site to the remainder of a molecule. Thus, for example, (f)

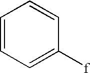

may have the meaning of 2-tolyl, 3-tolyl, 4-tolyl and benzyl.

The style used, in which in group

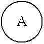

a bond of a substituent is shown towards the centre of the group A, denotes, unless otherwise stated, that this substituent may be bound to any free position of the group A carrying a H atom.

The term “optionally substituted” used in this application denotes that the group thus designated is either unsubstituted or mono- or polysubstituted by the substituents specified. If the group in question is polysubstituted, the substituents may be identical or different.

The groups and substituents described hereinbefore may, unless stated otherwise, be mono- or polysubstituted by fluorine. Preferred fluorinated alkyl groups are fluoromethyl, difluoromethyl and trifluoromethyl. Preferred fluorinated alkoxy groups are fluoromethoxy, difluoromethoxy and trifluoromethoxy. Preferred fluorinated alkylsulphinyl and alkylsulphonyl groups are trifluoromethylsulphinyl and trifluoromethylsulphonyl.

The compounds of general formula I according to the invention may have acid groups, predominantly carboxyl groups, and/or basic groups such as e.g. amino functions. Compounds of general formula I may therefore be present as internal salts, as salts with pharmaceutically useable inorganic acids such as hydrochloric acid, sulphuric acid, phosphoric acid, sulphonic acid or organic acids (such as for example maleic acid, fumaric acid, citric acid, tartaric acid, acetic acid or trifluoroacetic acid) or as salts with pharmaceutically useable bases such as alkali or alkaline earth metal hydroxides or carbonates, zinc or ammonium hydroxides or organic amines such as e.g. diethylamine, triethylamine, triethanolamine, inter alia.

The compounds according to the invention may be obtained using methods of synthesis which are known in principle, from starting compounds familiar to those skilled in the art (cf. for example: Houben Weyl—Methods of Organic Chemistry, Vol. E22, Synthesis of Peptides and Peptidomimetics, M. Goodman, A. Felix, L. Moroder, C. Toniolo Eds., Georg Thieme Verlag Stuttgart, New York). Provided that he knows their structure the skilled man will be able to synthesise the compounds according to the invention starting from known starting materials without any further instructions. Thus, the compounds may be obtained according to the preparation processes described in more detail hereinafter.

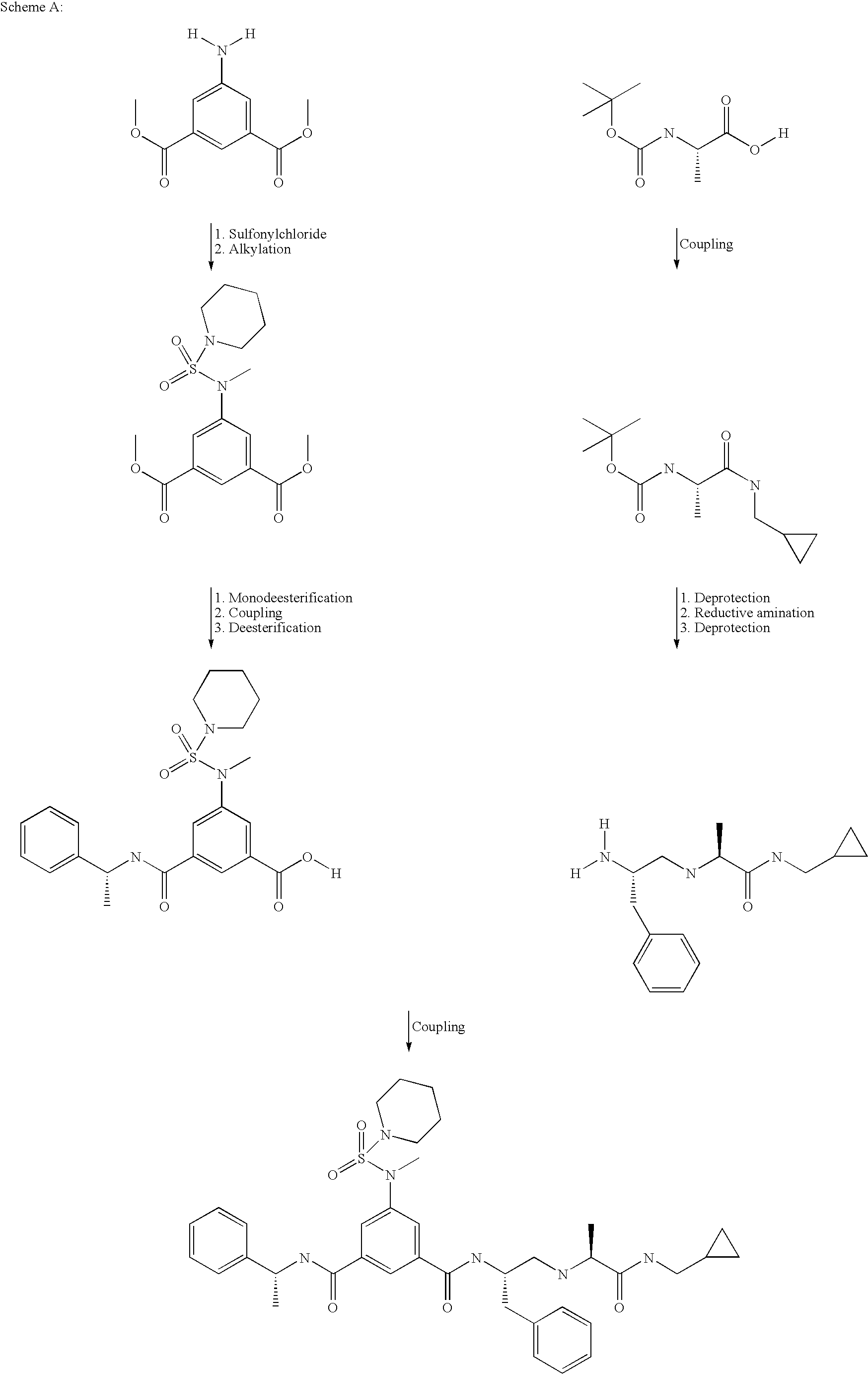

Diagram A illustrates by way of example the synthesis of the compounds according to the invention. Starting from a Boc-protected amino acid an amide is prepared by standard coupling methods. The amine obtained after deprotection has been carried out again is reductively aminated with a Boc-protected aminoaldehyde. The amine obtained after deprotection has been carried out again is coupled with an isophthalic acid monoamide component to obtain the end product.

In an alternative method of synthesis the compounds according to the invention may be prepared according to Scheme B:

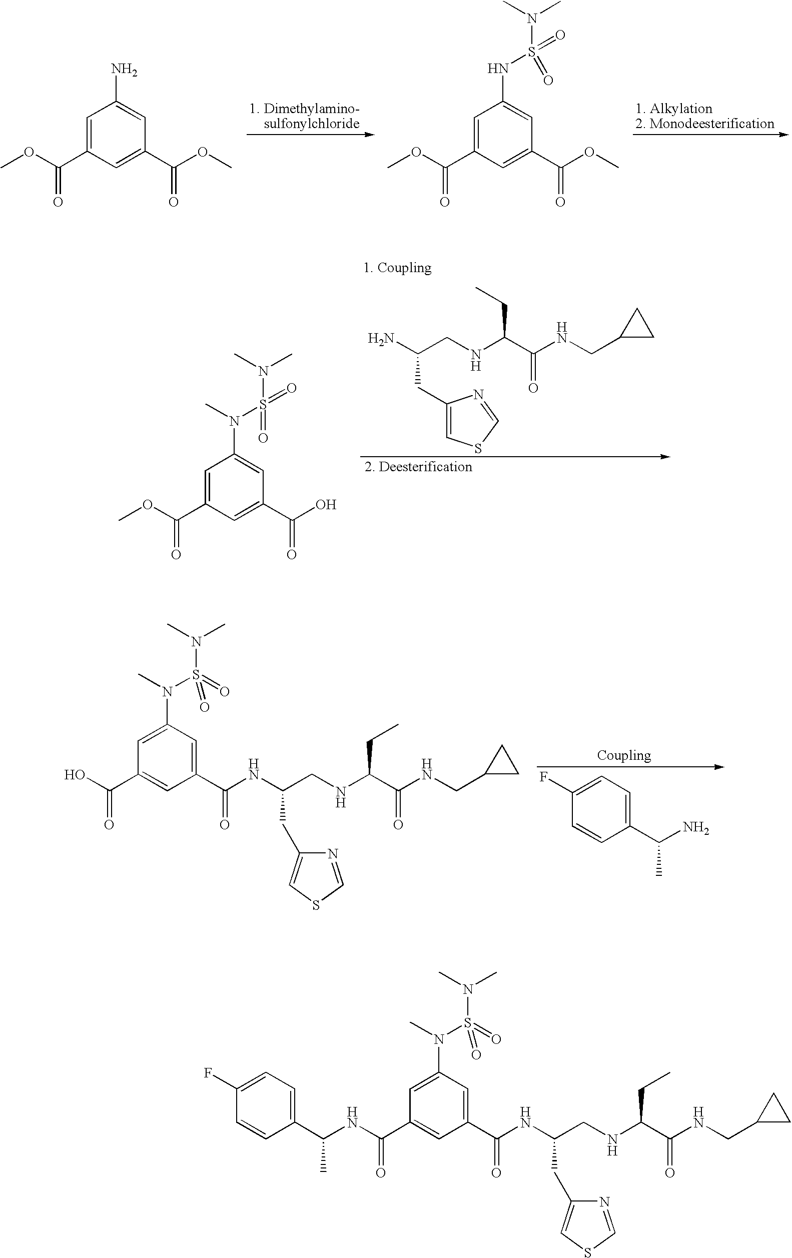

For this, aminoisophthalic acid diester is reacted with a corresponding sulphonic acid chloride, the sulphonamide nitrogen is alkylated and one of the two ester groups is cleaved. Then the compound is coupled to a dipeptide component which is prepared according to Scheme A by reductive amination, the ester function is saponified and the acid is coupled with a corresponding amine to produce the end product.

As stated previously, the compounds of formula (I) may be converted into the salts thereof, and particularly, for pharmaceutical use, into the physiologically and pharmacologically acceptable salts thereof. These salts may be present on the one hand as physiologically and pharmacologically acceptable acid addition salts of the compounds of formula (I) with inorganic or organic acids. On the other hand, in the case of acidically bound hydrogen, the compound of formula (I) may also be converted by reaction with inorganic bases into physiologically and pharmacologically acceptable salts with alkali or alkaline earth metal cations as counter-ion. The acid addition salts may be prepared for example using hydrochloric acid, hydrobromic acid, sulphuric acid, phosphoric acid, methanesulphonic acid, acetic acid, trifluoroacetic acid, fumaric acid, succinic acid, lactic acid, citric acid, tartaric acid or maleic acid.

Moreover mixtures of the above-mentioned acids may be used. For preparing the alkali and alkaline earth metal salts of the compound of formula (I) with acidically bound hydrogen it is preferable to use the alkali and alkaline earth metal hydroxides and hydrides, while the hydroxides and hydrides of the alkali metals, particularly of sodium and potassium, preferably sodium and potassium hydroxide, are particularly preferred.

The compounds of general formula (I) according to the invention and the corresponding pharmaceutically acceptable salts thereof are theoretically suitable for treating and/or preventatively treating all those conditions or diseases that are characterised by a pathological form of β-amyloid-peptide, such as for example β-amyloid-plaques, or that can be influenced by inhibiting β-secretase. For example the compounds according to the invention are particularly suitable for the prevention, treatment or for slowing down the progress of diseases such as Alzheimer's disease (AD) and other diseases associated with, die with abnormal processing of the Amyloid Precursor Protein (APP) or aggregation of Abeta peptide, as well as diseases that can be treated or prevented by inhibiting β-secretase or cathepsin D. Corresponding diseases include MCI (“mild cognitive impairment”), trisomy 21 (Down's syndrome), cerebral amyloidangiopathy, degenerative dementias, hereditary cerebral haemorrhage with amyloidosis—Dutch type (HCHWA-D), Alzheimer's dementia with Lewy bodies, trauma, stroke, pancreatitis, inclusion body myositis (IBM), as well as other peripheral amyloidoses, diabetes and arteriosclerosis.

The compounds are preferably suitable for the prevention and treatment of Alzheimer's disease. The compounds according to the invention may be used as a monotherapy and also in combination with other compounds that can be administered for the treatment of the above mentioned diseases.

The compounds according to the invention are particularly suitable for use in mammals, preferably primates, particularly preferably humans, for the treatment and/or prevention of the above mentioned conditions and diseases.

The compounds according to the invention may be administered orally, parenterally (by intravenous, intramuscular route, etc.), by intranasal, sublingual, inhalative, intrathecal, topical or rectal route.

In the case of the preferred oral administration, the compounds according to the invention may be formulated such that the compounds according to the invention do not come into contact with the acidic gastric juices. Suitable oral formulations may for example have gastric juice-resistant coatings which only release the active substances in the small bowel. Such tablet coatings are known to the skilled man.

Suitable pharmaceutical formulations for administering the compounds according to the invention are for example tablets, pellets, coated tablets, capsules, powders, suppositories, solutions, elixirs, active substance plasters, aerosols and suspensions.

About 0.1 to 1000 mg of one of the compounds according to the invention or of a mixture of several of these compounds are formulated on their own or together with pharmaceutically conventional excipients such as carriers, diluents, binders, stabilisers, preservatives, dispersants etc. To form a dosage unit in a manner known to those skilled in the art.

A dosage unit (e.g. Tablet) preferably contains between 2 and 250 mg, particularly preferably between 10 and 100 mg of the compounds according to the invention.

Preferably the pharmaceutical formulations are administered 1, 2, 3 or 4 times, particularly preferably once or twice, most preferably once a day.

The dosage required to achieve the corresponding activity for treatment or prevention usually depends on the compound which is to be administered, the patient, the nature and gravity of the illness or condition and the method and frequency of administration and is for the patient's doctor to decide.

Expediently, the amount of the compounds according to the invention administered is in the range from 0.1 to 1000 mg/day, preferably 2 to 250 mg/day, particularly preferably 5 to 100 mg/day when administered orally. For this purpose, the compounds of formula (I) prepared according to the invention may be formulated, optionally with other active substances, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as tablets, pellets, coated tablets, capsules, powders, suppositories, solutions, elixirs, active substance plasters, aerosols and suspensions.

The compounds according to the invention may also be used in conjunction with other active substances, particularly for the treatment and/or prevention of the diseases and conditions mentioned above. Other active substances which are suitable for such combinations include, in particular, those which potentiate the therapeutic effect of a compound according to the invention with respect to one of the indications mentioned and/or which allow the dosage of a compound according to the invention to be reduced. Therapeutic agents which are suitable for such a combination include, for example, beta-secretase inhibitors; gamma-secretase inhibitors; amyloid aggregation inhibitors such as e.g. alzhemed; directly or indirectly acting neuroprotective substances; antioxidants such as e.g. Vitamin E or ginkgolides; anti-inflammatory substances such as e.g. Cox inhibitors, NSAIDs with additionally or solely Aβ lowering properties; HMG-CoA reductase inhibitors (statins); acetylcholinesterase inhibitors such as donepezil, rivastigmine, tacrine, galantamine; NMDA receptor antagonists such as e.g. memantine; AMPA agonists; substances that modulate the concentration or release of neurotransmitters such as NS-2330; substances that induce the secretion of growth hormone such as ibutamoren mesylate and capromorelin; CB-1 receptor antagonists or inverse agonists; antibiotics such as minocycline or rifampicin; PDE-IV and PDE-IX inhibitors, GABA_{A} inverse agonists, nicotine agonists, histamine H3 antagonists, 5 HT-4 agonists or partial agonists, 5HT-6 antagonists, a2-adrenoreceptor antagonists, muscarinic M1 agonists, muscarinic M2 antagonists, metabotropic glutamate-receptor 5 positive modulators, as well as other substances that modulate receptors or enzymes in a manner such that the efficacy and/or safety of the compounds according to the invention is increased and/or unwanted side effects are reduced.

Preferred combinations are those comprising one or more of the compounds according to the invention with one or more of the following substances selected from among Alzhemed, Vitamin E, ginkgolides, donepezil, rivastigmine, tacrine, galantamine, memantine, NS-2330, ibutamoren mesylate, capromorelin, minocycline and/or rifampicin.

The compounds according to the invention, or the physiologically acceptable salts thereof, and the other active substances to be combined therewith, may be present together in one dosage unit, for example a tablet or capsule, or separately in two identical or different dosage units, for example as a so-called kit-of-parts.

The compounds according to the invention may also be used in conjunction with immunotherapies such as e.g. active immunisation with Abeta or parts thereof or passive immunisation with humanised anti-Abeta antibodies for the treatment of the above mentioned diseases and conditions.

The dosage for the combination partners mentioned above is usefully ⅕ of the lowest dose normally recommended up to 1/1 of the normally recommended dose.

Therefore, in another aspect, this invention relates to the use of a compound according to the invention or a physiologically acceptable salt of such a compound combined with at least one of the active substances described above as a combination partner, for preparing a pharmaceutical composition which is suitable for the treatment or prevention of diseases or conditions which can be affected by inhibiting β-secretase.

The use of the compound according to the invention, or a physiologically acceptable salt thereof, in combination with another active substance may take place simultaneously or at staggered times, but particularly within a short space of time. If they are administered simultaneously, the two active substances are given to the patient together; while if they are used at staggered times the two active substances are given to the patient within a period of less than or equal to 12 hours, but particularly less than or equal to 6 hours.

Consequently, in another aspect, this invention relates to a pharmaceutical composition which comprises a compound according to the invention or a physiologically acceptable salt of such a compound and at least one of the active substances described above as combination partners, optionally together with one or more inert carriers and/or diluents.

Thus, for example, a pharmaceutical composition according to the invention comprises a combination of a compound of formula (I) according to the invention or a physiologically acceptable salt of such a compound and at least one other of the above-mentioned active substances, optionally together with one or more inert carriers and/or diluents.

The compounds according to the invention inhibit the proteolysis of the APP protein between the amino acids Met595 and Asp596 (the numbering relates to the APP695 isoform) or the proteolysis of other APP isoforms such as APP751 and APP770 or mutated APP at the corresponding site, which is also referred to as the β-secretase cutting site. The inhibition of β-secretase should therefore lead to a decreased production of the β-amyloid peptide (Aβ).

The activity of β-secretase may be investigated in assays based on different detection technologies. In the test set-up a catalytically active form of β-secretase is incubated with a potential substrate in a suitable buffer. The reduction in the substrate concentration or the increase in product concentration may be achieved using various technologies depending on the substrate used: HPLS-MS analysis, fluorescence assays, fluorescence-quenching assays, luminescence assays are a non-representative selection of the different possibilities. Assay systems in which the effectiveness of a compound can be demonstrated are described e.g. In U.S. Pat. No. 5,942,400 and U.S. Pat. No. 5,744,346 and hereinafter. An alternative assay format comprises displacing a known β-secretase ligand with a test substance (US 2003/0125257).

The substrate used may be either the APP protein or parts thereof or any amino acid sequence that can be hydrolysed by the β-secretase. A selection of these sequences can be found e.g. In Tomasselli et al. 2003 in J. Neurochem 84: 1006. A peptide sequence of this kind may be coupled to suitable dyes that provide indirect evidence of proteolysis.

The enzyme source used may be the complete β-secretase enzyme or mutants with a catalytic activity or only parts of the β-secretase which still contain the catalytically active domain. Various forms of β-secretase are known and available and may serve as an enzyme source in a corresponding test set-up. This includes the native enzyme and also the recombinant or synthetic enzyme. Human β-secretase is known by the name Beta Site APP Cleaving Enzyme (BACE), Asp2 and memapsin 2 and is described e.g. In U.S. Pat. No. 5,744,346 and in the patent applications WO 98/22597, WO 00/03819, WO 01/23533, and WO 00/17369, as well as in the scientific literature (Hussain et al., 1999, Mol. Cell. Neurosci. 14: 419-427; Vassar et. al., 1999, Science 286: 735-741; Yan et al., 1999, Nature 402: 533-537; Sinha et. al., 1999, Nature 40: 537-540; and Lin et. al., 2000, PNAS USA 97: 1456-1460). Synthetic forms of the enzyme have also been described (WO 98/22597 and WO 00/17369). β-Secretase may be extracted and purified from human brain tissue, for example, or produced recombinantly in mammalian cell cultures, insect cell cultures, yeasts or bacteria.

To calculate the IC50 value of a substance, different amounts of substance are incubated with the β-secretase in an assay. The IC50 value of a compound is defined as the substance concentration at which a 50% reduction in the detected signal is measured by comparison with the mixture without any test compound. Substances are evaluated as having an inhibiting effect on β-secretase if under these conditions their IC50 value is less than 50 μM, preferably less than 10 μM, particularly preferably less than 1 μM and most particularly preferably less than 100 nM.

An assay for detecting β-secretase activity may have the following appearance, in detail:

The ectodomain of BACE (amino acids 1-454) fused to the recognition sequence for an anti-Myc antibody and a poly-histidine is secreted overnight by HEK293/APP/BACE_{ect}. Cells in OptiMEM® (Invitrogen). A 10 μl aliquot of this cell culture supernatant serves as an enzyme source. The enzyme is stable over more than 3 months when stored at 4° C. or −20° C. in OptiMEM®. The substrate used is a peptide with the amino acid sequence SEVNLDAEFK to which the Cy3 fluorophore (Amersham) is coupled N-terminally and the Cy5Q fluorophore (Amersham) is coupled C-terminally. The substrate is dissolved in DMSO in a concentration of 1 mg/ml and used in the test in a concentration of 1 μM. In addition the test mixture contains 20 mM NaOAc, pH 4.4, and at most 1% DMSO. The test is carried out in a 96-well dish in an overall volume of 200 μl over 30 minutes at 30° C. The cleaving of the substrate is recorded kinetically in a fluorimeter (ex: 530 nm, em: 590 nm). The assay is started by the addition of the substrate.

As controls, mixtures with no enzyme or with no inhibitor are included on each dish. The IC₅₀ value for the test compound is calculated using standard software (e.g. GraphPad Prism®) from the percentage inhibition of the substance at different test concentrations. The relative inhibition is calculated from the reduction in the signal intensity in the presence of the substance based on the signal intensity without the substance.

The compounds (1)-(46) mentioned in the Table hereinbefore have IC₅₀ values of less than 30 μM, measured using the test described above.

The activity of the β-secretase may also be investigated in cellular systems. As APP is a substrate for β-secretase and Aβ is secreted by the cells after the processing of APP by β-secretase, cellular test systems for detecting β-secretase activity are based on detecting the amount of Aβ formed over a defined period of time.

The selection of suitable cells comprises, but is not restricted to, human embryonic kidney fibroblasts 293 (HEK293), Chinese Hamster Ovary cells (CHO), human H4 neuroglioma cells, human U373 MG astrocytoma glioblastoma cells, neuroblastoma N2a cells in the mouse, which stably or transiently express APP or mutated forms of APP, such as e.g. The Swedish or London or Indiana Mutation. The transfection of the cells is carried out for example by cloning the cDNA of human APP into an expression vector such as e.g. PcDNA3 (Invitrogen) and adding it to the cells with a transfection reagent such as e.g. Lipofectamine (Invitrogen) according to the manufacturer's instructions.

The secretion of Aβ may also be measured from cells without genetic modification using a suitably sensitive Aβ detection assay such as e.g. ELISA or HTRF. Cells that may be used for this are, besides other cells, human IMR32 neuroblastoma cells, for example.

The secretion of Aβ may also be investigated in cells obtained from the brains of embryos or the young of APP transgenic mice, such as e.g. In those obtained by Hsiao et al 1996 Science 274: 99-102, or from other organisms such as e.g. guinea pigs or rats.

Substances are evaluated as having an inhibiting effect on β-secretase if under these conditions their IC50 value is less than 50 μM, preferably less than 10 μM, particularly preferably less than 1 μM and most particularly preferably less than 100 nM.

An example of the procedure for carrying out a cell assay is described below: U373-MG cells which stably express APP (isoform 751) are cultivated in a culture medium such as DMEM+glucose, sodium pyruvate, glutamine and 10% FCS at 37° C. in a steam-saturated atmosphere containing 5% CO₂. In order to investigate the β-secretase inhibiting activity of substances, the cells are incubated with different concentrations of the compound between 50 μM and 50 pM for 12-24 h. The substance is dissolved in DMSO and is diluted for the assay in culture medium so that the DMSO concentration does not exceed 0.5%. The production of Aβ during this period is detected using an ELISA, which uses the antibodies 6E10 (Senentek) and SGY3160 (C. Eckman, Mayo Clinic, Jacksonville, Fla., USA) as capturing antibodies that are bound to the microtitre plate and Aβ40- and Aβ42-specific antibodies (Nanotools, Germany), coupled to alkaline phosphatase, as detecting antibodies. Non-specific binding of proteins to the microtitre plate is prevented by blocking with Block Ace (Serotec) before the addition of the Aβ-containing culture supernatant. The quantifying of the amounts of Aβ contained in the cell supernatant is carried out by adding the substrate for alkaline phosphatase CSPD/Sapphire II (Applied Biosystems) according to the manufacturer's instructions. Possible non-specific effects of the test compound on the vitality of the cells are excluded by determining precisely these effects by AlamarBlue (resazurin) reduction over a period of 60 minutes.

The potency of non-toxic substances is determined by calculating the concentration that brings about a 50% reduction in the amount of Aβ secreted compared with untreated cells.

Moreover, different animal models may be used to investigate the β-secretase activity and/or the APP processing and the release of Aβ. Thus, for example, transgenic animals that express APP and/or β-secretase may be used to test the inhibitory activity of compounds of this invention. Corresponding transgenic animals are described for example in U.S. Pat. No. 5,877,399, U.S. Pat. No. 5,612,486, U.S. Pat. No. 5,387,742, U.S. Pat. No. 5,720,936, U.S. Pat. No. 5,850,003, U.S. Pat. No. 5,877,015 and U.S. Pat. No. 5,811,633, and in Games et al., 1995, Nature 373: 523. Preferably, animal models are used that display some of the characteristics of AD pathology. The administering of β-secretase inhibitors according to this invention and the subsequent investigation of the pathology of the animals constitutes a further alternative method of demonstrating β-secretase inhibition using the compounds. The compounds are administered in such a way that they can reach their intended site of activity in a pharmaceutically effective form and quantity.

The test for detecting cathepsin D (EC: 3.4.23.5) inhibition was carried out as follows: 20 mU of recombinant cathepsin D (Calbiochem, Cat. No. 219401) in 20 mM sodium acetate puffer pH 4.5 with 5 μM substrate peptide and different concentrations of the test substance are incubated at 37° C. in a 96-well dish and the conversion is recorded for 60 minutes in a fluorimeter (emission: 535 nm, extinction: 340 nm). The peptide substrate used has the following sequence: NH₂-Arg-Glu(Edans)-Glu-Val-Asn-Leu-Asp-Ala-Glu-phe-Lys(Dabcyl)-Arg-COON (Bachem). However, a peptide or protein substrate with a sequence that can be cleaved proteolytically from Cathepsin D may also be used. The test substances are dissolved in DMSO and are used in the assay after dilution to a maximum of 1% DMSO.

The assay is started by the addition of the substrate.

As controls, mixtures with no enzyme or with no inhibitor are included on each dish.

The IC₅₀ value for the test compound is calculated using standard software (e.g. GraphPad Prism®) from the percentage inhibition of the substance at different test concentrations. The relative inhibition is calculated from the reduction in the signal intensity in the presence of the substance based on the signal intensity without the substance.

The compounds (1)-(46) mentioned in the Table hereinbefore exhibited an inhibitory effect on cathepsin D in the test described here.

The following Examples are intended to illustrate the invention, without restricting it.

### EXAMPLES

The following abbreviations are used in the descriptions of the tests:

BOC tert.-butoxycarbonyl

### DIPEA N-ethyl-diisopropylamine

DMF dimethylformamide
ES-MS electrospray-mass spectrometry
HPLC high pressure liquid chromatography
HPLC-MS high pressure liquid chromatography with mass detection
sat. saturated
HOBt 1-hydroxy-benzotriazole-hydratee
i.vac. in vacuo
conc. concentrated
MPLC medium pressure liquid chromatography
RT retention time
TBTU O-(benzotriazol-1-yl)-N,N,N′,N′-tetramethyluronium tetrafluoroborate
TEA triethylamine
TFA trifluoroacetic acid
THF tetrahydrofuran
→* indicates the binding site of a group

The HPLC 1 data were generated under the following conditions:

Waters Alliance 2695 HPLC, Waters 2700 Autosampler, Waters 2996 Diode array detector

The eluant used was as follows:

A: water with 0.13% TFA
B: acetonitrile with 0.10% TFA

| time in min | % A | % B | flow rate in ml/min |
|---|---|---|---|
| 0.00 | 95 | 5 | 1.00 |
| 0.75 | 95 | 5 | 1.00 |
| 5.25 | 2 | 98 | 1.00 |
| 5.75 | 2 | 98 | 1.00 |
| 6.05 | 95 | 5 | 1.00 |
| 6.55 | 95 | 5 | 1.00 |

The stationary phase used was a Varian column, Microsorb 100 C₁₈ 3 μm, 4.6 mm×50 mm, batch no. 2231108 (column temperature: constant at 25° C.).

The diode array detection took place in the wavelength range from 210-300 nm.

The HPLC-MS data were generated under the following conditions:

Waters ZMD, Waters Alliance 2690 HPLC, Waters 2700 Autosampler, Waters 996 diode array detector

The eluant used was as follows:

A: water with 0.13% TFA
B: acetonitrile with 0.10% TFA

| time in min | % A | % B | flow rate in ml/min |
|---|---|---|---|
| 0.0 | 95 | 5 | 1.00 |
| 0.1 | 95 | 5 | 1.00 |
| 3.1 | 2 | 98 | 1.00 |
| 4.5 | 2 | 98 | 1.00 |
| 5.0 | 95 | 5 | 1.00 |

The stationary phase used was a Waters column, Xterra MS C₁₈ 2.5 μm, 4.6 mm×30 mm, (column temperature: constant at 25° C.).

The diode array detection took place in the wavelength range from 210-500 nm.

### Example 1

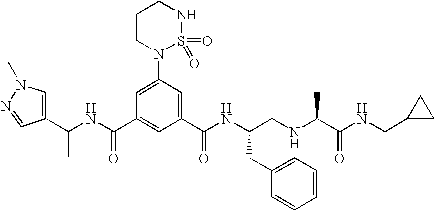

### a) Preparation of 1-a:

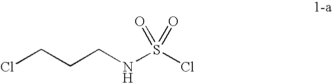

1.3 ml (15.4 mmol) sulphuryl chloride were metered in to a solution of 1.0 g (7.7 mmol) 3-chloro-propylamine-hydrochloride in 10 ml acetonitrile while cooling with an ice bath and stirred overnight at 85° C. Then the reaction solution was evaporated down i. vac. This produced a quantitative yield of 1-a.

### b) Preparation of 1-b:

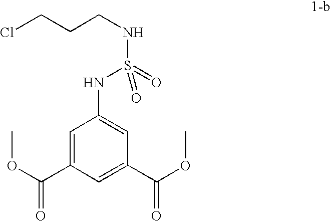

1.0 g (4.8 mmol) dimethyl 5-amino-isophthalate were suspended in 10 ml of pyridine and slowly combined with 1.5 g (7.8 mmol) 1-a and stirred overnight at ambient temperature. Then the reaction solution was combined with dichloromethane and washed with 1N HCl and water, the organic phase was separated off using a phase separation cartridge and evaporated down i. vac. This yielded 1.1 g (41%) brown crystals 1-b.

RT (HPLC 1)=4.51 min

### c) Preparation of 1-c:

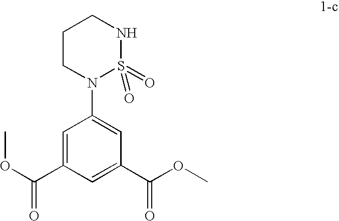

10.86 g (29.8 mmol) 1-b were dissolved in 100 ml DMF, combined with 6.85 g (61.0 mmol) potassium-tert-butoxide and stirred overnight at 60° C. Then the reaction solution was combined with water and extracted with dichloromethane. The combined organic phases were dried on MgSO₄, filtered and the filtrate was evaporated to dryness i. vac. The residue was purified by MPLC with the eluant (ethyl acetate/heptane 7:3 to pure methanol). This yielded 2.65 g (27%) 1-c as yellowish crystals.

ES-MS (M+H)⁺=329

RT (HPLC 1)=4.29 min

### d) Preparation of 1-d:

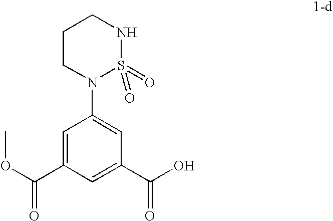

2.65 g (8.1 mmol) 1-c were dissolved in 50 ml of methanol and 50 ml THF, at 0° C. 8.0 ml (8.0 mmol) 1N NaOH were added and the reaction solution was stirred for 7 hours at ambient temperature. Then the solvent was eliminated using the rotary evaporator, the residue was dissolved in 30 ml 1N HCl and extracted with ethyl acetate. The combined organic phases were dried and purified by chromatography on silica gel with the eluant (dichloromethane/methanol 80:20). This yielded 1.3 g (51%) white crystals 1-d.

RT (HPLC 1)=3.79 min

### e) Preparation of 1-e:

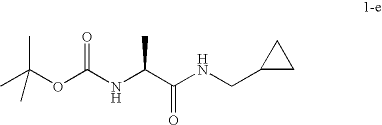

9.46 g (50.0 mmol) Boc-L-alanine in 120 ml dichloromethane were combined with 16.1 g (50.0 mmol) TBTU and 25.5 ml (15.0 mmol) DIPEA while cooling with an ice bath, then 5.38 g (50.0 mmol) cyclopropylmethylamine-hydrochloride were added. The reaction solution was stirred for 5 hours at ambient temperature and then extracted with 20% KHCO₃ solution and water. The organic phases were separated using a phase separation cartridge and evaporated to dryness i. vac. This yielded 12.8 g (95%) of a colourless oil 1-e.

RT (HPLC-MS)=2.48 min

### f) Preparation of 1-f:

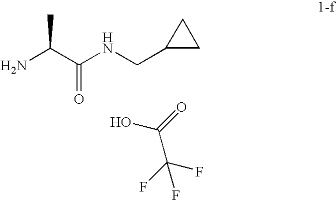

29.0 g (0.1 mol) 1-e was dissolved in 130 ml dichloromethane and combined with 100 ml (1.3 mol) trifluoroacetic acid. The reaction solution was stirred for 1 h at ambient temperature, then evaporated to dryness using the rotary evaporator. This gave a quantitative yield of 1-f as a yellow oil.

### g) Preparation of 1-g:

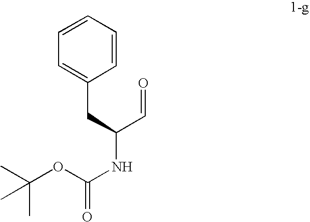

29.7 g (70.0 mmol) Dess-Martin-periodinane were suspended in 150 ml dichloromethane, then within 40 minutes a solution of 16.0 g (63.7 mmol) Boc-phenylalaninol in 150 ml dichloromethane was metered in. The reaction solution was stirred for 2 hours at ambient temperature, then combined with 200 ml 20% KHCO₃ solution and 200 ml 10% Na₂S₂O₃ solution. The mixture was stirred for 20 min at ambient temperature, the phases were separated and the organic phase was washed with 20% KHCO₃ solution and water. The organic phase was dried and evaporated to dryness using the rotary evaporator. This gave a quantitative yield of 1-g as white crystals.

### h) Preparation of 1-h:

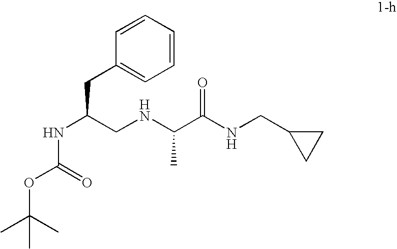

15.4 g (61.2 mmol) 1-f were dissolved in 200 ml acetonitrile and combined with 10.5 ml (61.2 mmol) DIPEA. The mixture was stirred for 10 min at ambient temperature, 15.3 g (61.2 mmol) 1-g were added and then cooled to 0° C. Then the reaction solution was combined with 7.0 ml (122 mmol) acetic acid and 20.5 g (91.8 mmol) sodium triacetoxyborohydride and left overnight at ambient temperature with stirring. The reaction solution was evaporated to dryness using the rotary evaporator and the residue was combined with dichloromethane and 1N NaHCO₃ solution. The phases were separated, the organic phase was dried and evaporated to dryness i. vac. The residue was purified by chromatography on silica gel with the eluant (ethyl acetate/heptane 7:3 to ethyl acetate/heptane 1:0). This yielded 13.1 g (43%) light yellow crystals 1-h.

RT (HPLC 1)=4.36 min

ES-MS (M+H)⁺=376

### i) Preparation of 1-i:

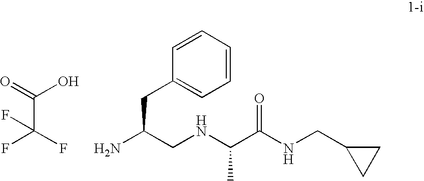

1-i was prepared analogously to Example 1-f from 1-h.

RT (HPLC 1)=3.37 min

### j) Preparation of 1-j:

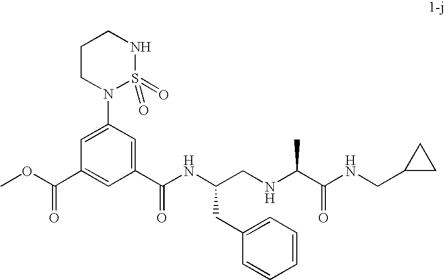

1-j was prepared analogously to 1-e from 1-d and 1-i.

RT (HPLC-MS)=2.55 min

(M+H)⁺ (HPLC-MS)=573

### k) Preparation of 1-k:

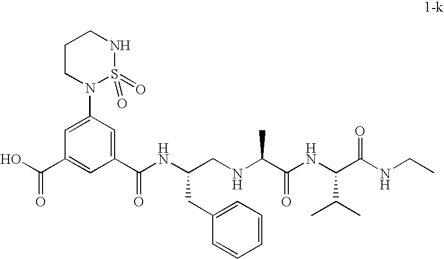

1-k was prepared analogously to 1-d from 1-j.

RT (HPLC 1)=4.03 min

ES-MS (M+H)⁺=556

### l) Preparation of 1-l:

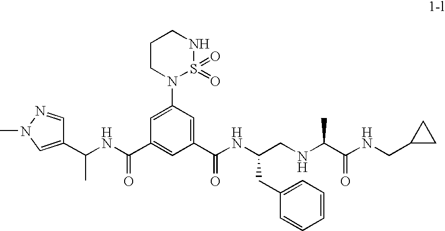

1-l was prepared analogously to 1-e from 1-k and 1-(1-methyl-1H-pyrazol-4-yl)-ethylamine.

RT (HPLC 1)=4.08 min

ES-MS (M+H)⁺=665

The following compounds were prepared analogously to 1-l from 1-k and the corresponding amount of amine:

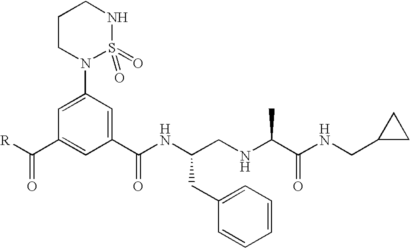

| Example | R | |
|---|---|---|
| 1.1 | 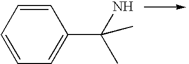 | RT (HPLC 1) = 4.72 min ES-MS (M − H)⁺ = 673 |
| 1.2 | 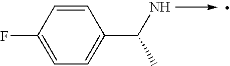 | RT (HPLC 1) = 4.62 min ES-MS (M + H)⁺ = 679 |
| 1.3 | 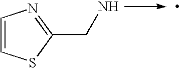 | RT (HPLC 1) = 4.09 min ES-MS (M + H)⁺ = 654 |

The following compounds were prepared analogously to Example 1 by using the corresponding educts:

| Example | | |
|---|---|---|
| 1.4 | 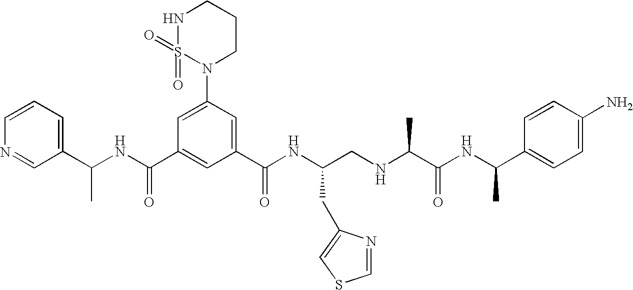 | RT (HPLC-MS) = 1.91 min. ES-MS (M + H)⁺ = 734 |
| 1.5 | 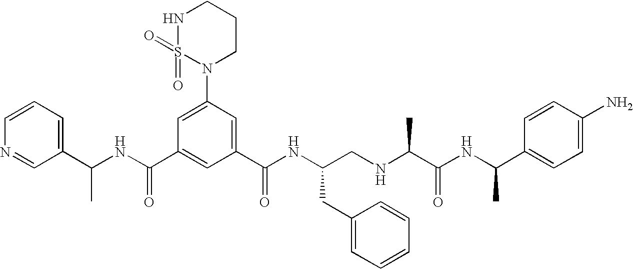 | RT (HPLC-MS) = 2.01 min. ES-MS (M + H)⁺ = 727 |
| 1.6 | 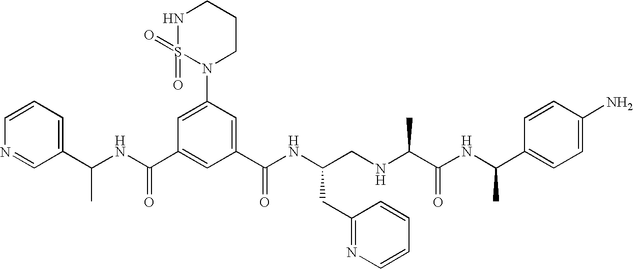 | RT (HPLC-MS) = 1.77 min. ES-MS (M + H)⁺ = 728 |
| 1.7 | 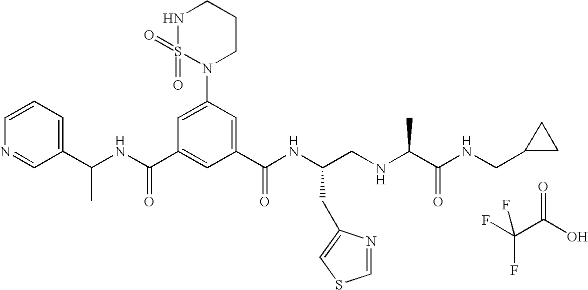 | RT (HPLC-1) = 3.56 min. ES-MS (M + H)⁺ = 669 |
| 1.8 | 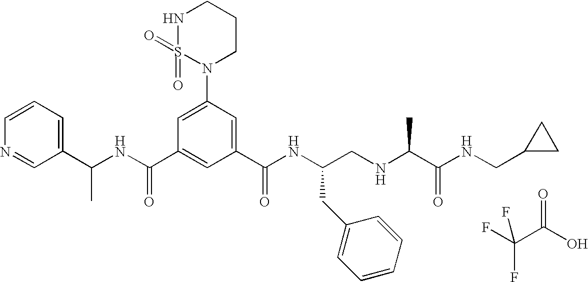 | RT (HPLC-1) = 3.80 min. ES-MS (M + H)⁺ = 662 |
| 1.9 | 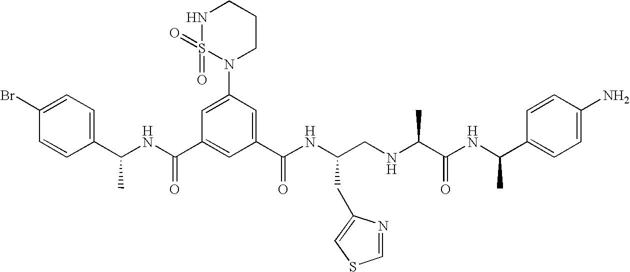 | RT (HPLC-MS) = 2.49 min. ES-MS (M + H)⁺ = 811/813 (Br) |
| 1.10 | 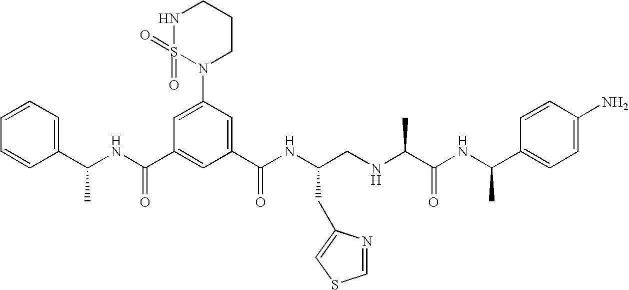 | RT (HPLC-MS) = 2.37 min. ES-MS (M + H)⁺ = 733 |
| 1.11 | 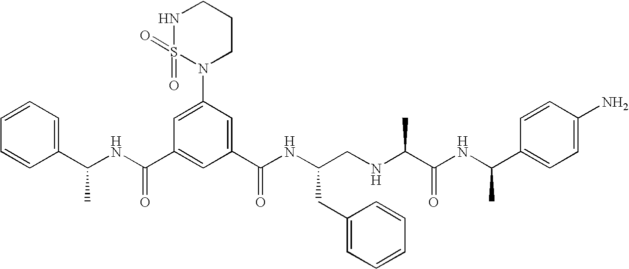 | RT (HPLC-MS) = 2.49 min. ES-MS (M + H)⁺ = 726 |
| 1.12 | 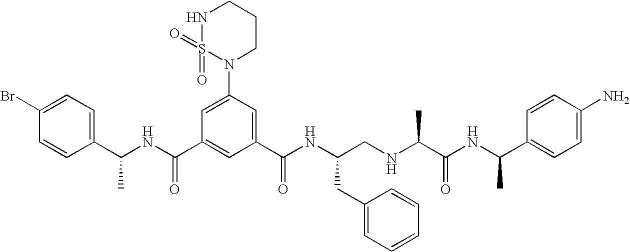 | RT (HPLC-MS) = 2.59 min. ES-MS (M + H)⁺ = 804/806 (Br) |
| 1.13 | 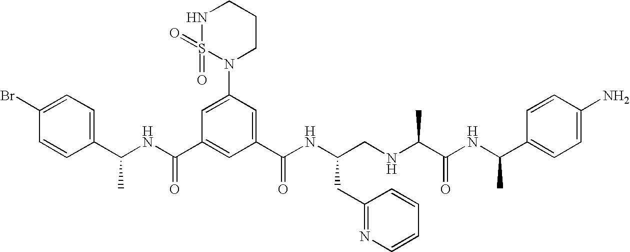 | RT (HPLC-MS) = 2.36 min. ES-MS (M + H)⁺ = 805/807 (Br) |
| 1.14 | 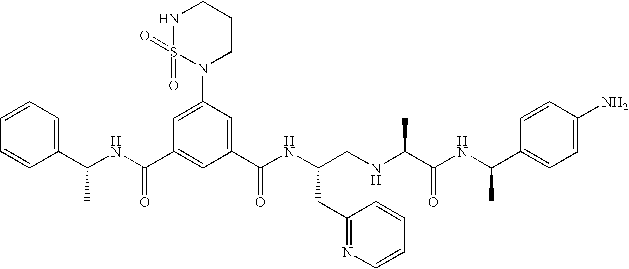 | RT (HPLC-1) = 3.80 min. ES-MS (M + H)⁺ = 727 |
| 1.15 | 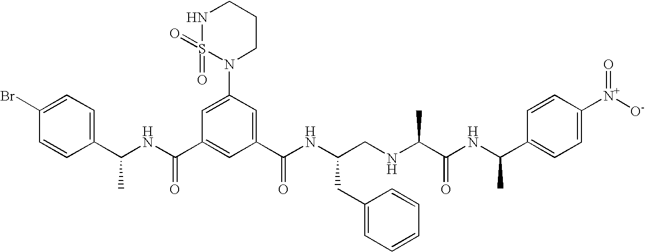 | RT (HPLC-1) = 4.98 min. ES-MS (M + H)⁺ = 834/836 (Br) |
| 1.16 | 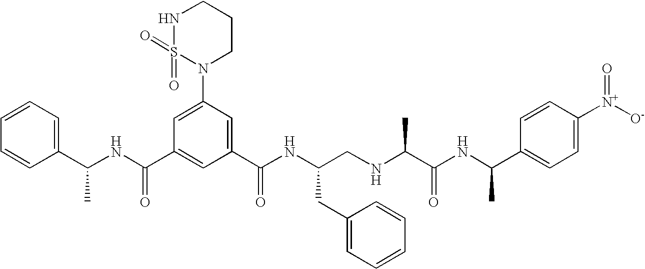 | RT (HPLC-1) = 4.82 min. ES-MS (M + H)⁺ = 756 |
| 1.17 | 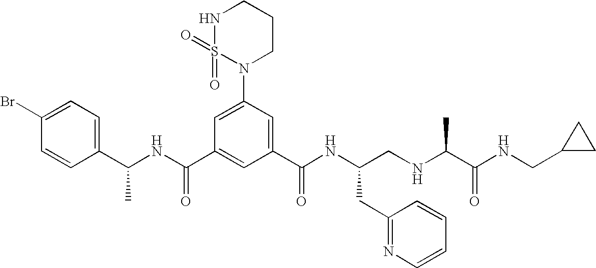 | RT (HPLC-1) = 4.30 min. ES-MS (M + H)⁺ = 740/742 (Br) |
| 1.18 | 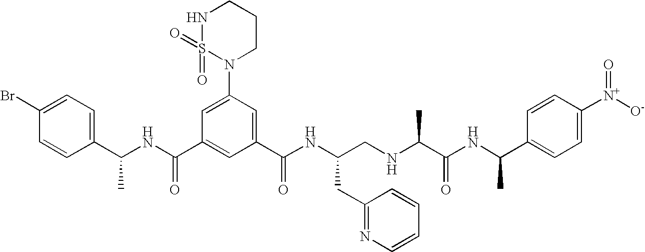 | RT (HPLC-1) = 4.51 min. ES-MS (M + H)⁺ = 835/837 (Br) |
| 1.19 | 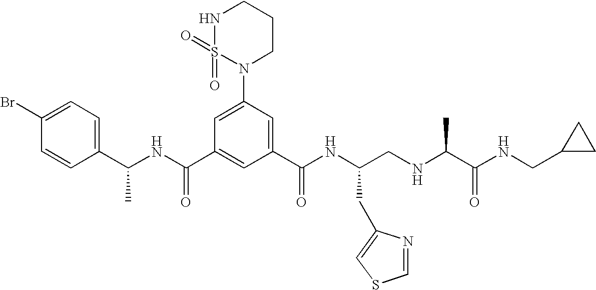 | RT (HPLC-1) = 4.55 min. ES-MS (M + H)⁺ = 746/748 (Br) |
| 1.20 | 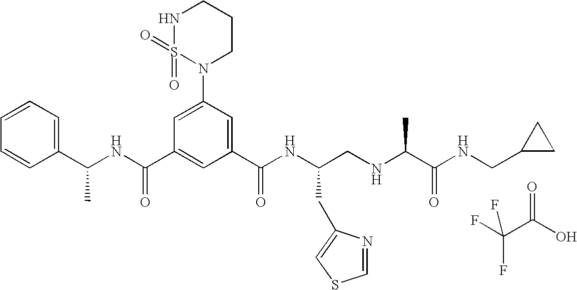 | RT (HPLC-1) = 4.34 min. ES-MS (M + H)⁺ = 668 |
| 1.21 | 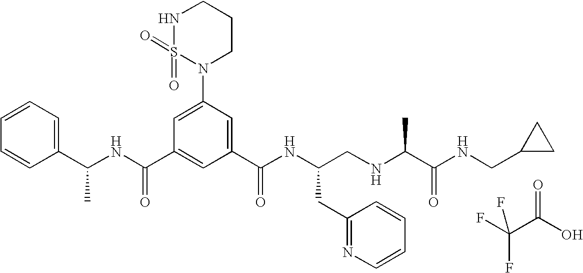 | RT (HPLC-1) = 3.98 min. ES-MS (M + H)⁺ = 662 |
| 1.22 | 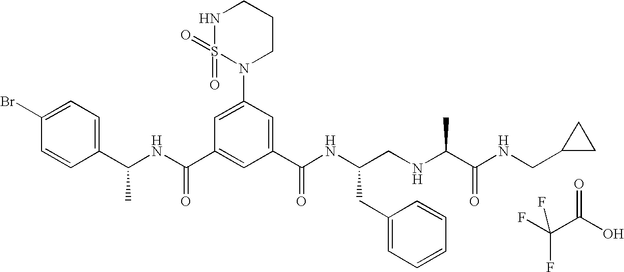 | RT (HPLC-1) = 4.79 min. ES-MS (M + H)⁺ = 739/741 (Br) |
| 1.23 | 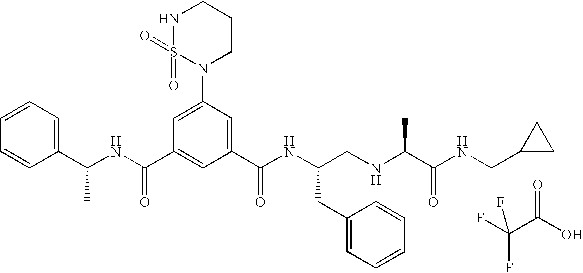 | RT (HPLC-1) = 4.63 min. ES-MS (M + H)⁺ = 661 |

### Example 2

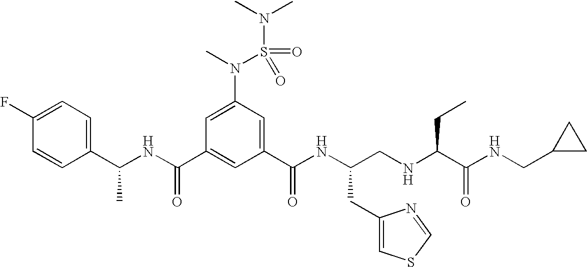

### a) Preparation of 2-a:

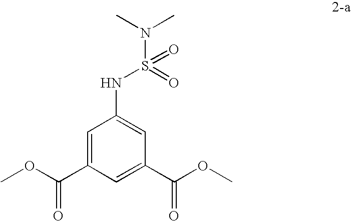

15 g (70.3 mmol) dimethyl 5-amino-isophthalate were dissolved in 150 ml of pyridine. The reaction solution was cooled to 0° C., at this temperature 12.0 ml (111.7 mmol) dimethylaminosulphonyl chloride were metered in, the mixture was heated to 90° C. and stirred for 12 h. Then it was poured onto 200 ml 4N HCl and the crystals precipitated were suction filtered. After extraction with diethyl ether and suction filtering again, they were dried in the drying cupboard at 40° C. and 17.9 g (64%) whitish crystals 2-a were obtained.

RT (HPLC 1)=4.14 min

### b) Preparation of 2-b:

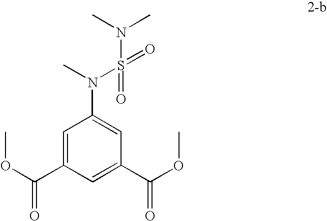

First 17.9 g (56.6 mmol) 2-a and then 9.3 ml (124.5 mmol) methyl iodide were added to a solution of 5.0 g (125 mmol) sodium hydride (60% in mineral oil) in 500 ml DMF. The reaction solution was stirred for 1 h at ambient temperature, combined with 500 ml of water and extracted with ethyl acetate. The combined organic phases were dried and evaporated to dryness using the rotary evaporator. This yielded 12.5 g (57%) 2-b as brown crystals.

RT (HPLC 1)=4.67 min

### c) Preparation of 2-c:

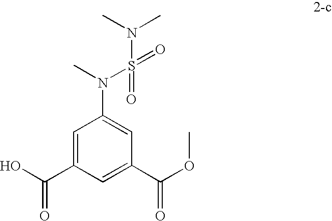

2-c was obtained analogously to 1-d from 2-b.

RT (HPLC-MS)=2.58 min

(M+H)⁺ (HPLC-MS)=318

### d) Preparation of 2-d:

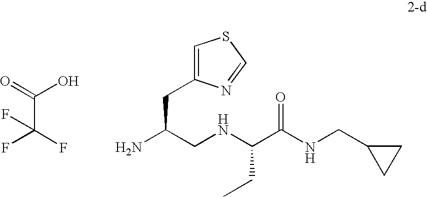

2-d was prepared analogously to 1-i, by substituting Boc-L-alanine by Boc-L-aminobutyric acid (step 1e) and Boc-phenyl-alaninol by Boc-L-thiazol-4-yl-alaninol (step 1g).

RT (HPLC-MS)=1.85 min

(M+H)⁺ (HPLC-MS)=298

### e) Preparation of 2-e:

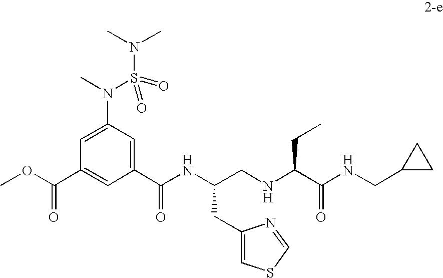

2-e was prepared analogously to 1-j from 2-c and 2-d.

RT (HPLC-MS)=2.54 min

(M+H)⁺ (HPLC-MS)=596

### f) Preparation of 2-f:

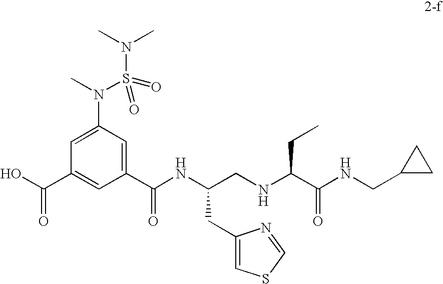

2-f was prepared analogously to 1-k from 2-e.

RT (HPLC 1)=3.95 min

ES-MS (M−H)⁺=579

### g) Preparation of 2-g:

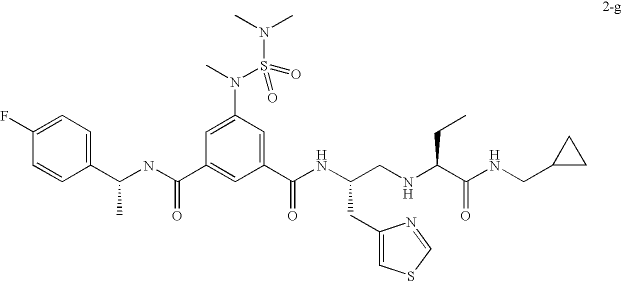

2-g was prepared analogously to 1-l from 2-f and (R)-1-(4-fluoro-phenyl)-ethylamine.

RT (HPLC 1)=4.62 min

ES-MS (M+H)⁺=702

The following compounds were prepared analogously to 2-g from 2-f and the corresponding amount of amine:

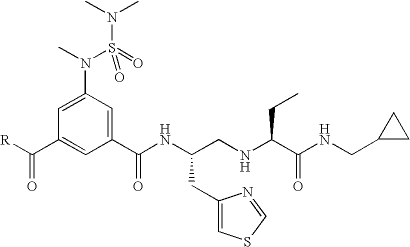

| Exam- | | |
|---|---|---|
| ple | R | |
| 2.1 | 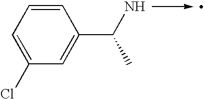 | RT (HPLC 1) = 4.82 min ES-MS (M + H)⁺ = 718/720 (Cl) |
| 2.2 | 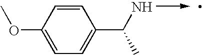 | RT (HPLC 1) = 4.57 min ES-MS (M + H)⁺ = 714 |
| 2.3 | 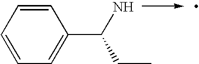 | RT (HPLC 1) = 4.74 min ES-MS (M + H)⁺ = 698 |
| 2.4 | 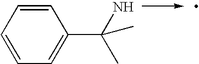 | RT (HPLC 1) = 4.68 min ES-MS (M + H)⁺ = 698 |
| 2.5 | 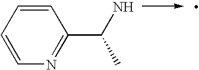 | RT (HPLC 1) = 3.83 min ES-MS (M + H)⁺ = 685 |
| 2.6 | 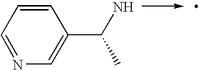 | RT (HPLC 1) = 3.78 min ES-MS (M + H)⁺ = 685 |
| 2.7 | 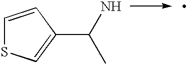 | RT (HPLC 1) = 4.55 min ES-MS (M + H)⁺ = 690 |

The following compounds were prepared analogously to 2-g from an amine analogous to 2-d, which was prepared by substituting Boc-L-alanine by Boc-L-aminobutyric acid (step 1e) and Boc-phenyl-alaninol by Boc-D-phenyl-alaninol (step 1g). The amine components used for the last step were (R)-1-phenyl-ethylamine or (R)-1-(3-chloro-phenyl)-ethylamine:

| Example | | |
|---|---|---|
| 2.8 | 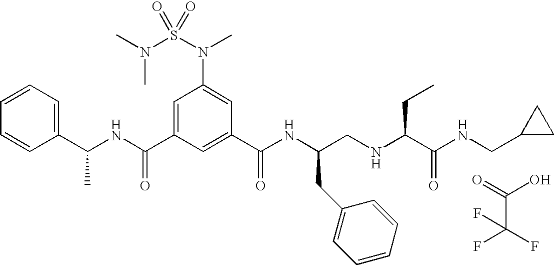 | RT (HPLC-MS) = 2.83 min. ES-MS (M + H)⁺ = 677 |
| 2.9 | 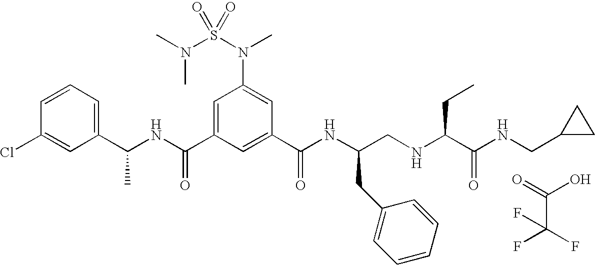 | RT (HPLC-MS) = 2.93 min. ES-MS (M + H)⁺ = 711/713 (Cl) |

The following compound was obtained analogously to 2-g from an amine analogous to 2-d, which was prepared by substituting Boc-L-alanine by Boc-L-aminobutyric acid (step 1e) and Boc-phenyl-alaninol by BOC-L-3-thienylalaninol (step 1g). (R)-1-(3-chloro-phenyl)-ethylamine was used as the amine component for the last step:

| Example | | |
|---|---|---|
| 2.10 | 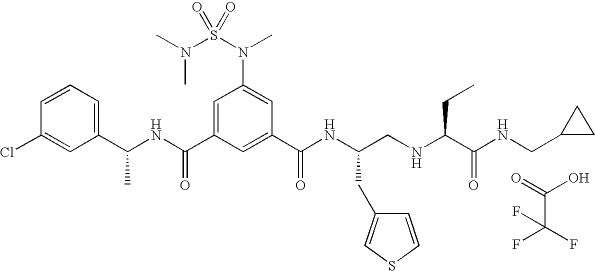 | RT (HPLC-MS) = 3.01 min. ES-MS (M + H)⁺ = 717/719 (Cl) |

The following compound was obtained analogously to 2-g from an amine analogous to 2-d, which was prepared by substituting Boc-L-alanine by Boc-L-aminobutyric acid (step 1e) and Boc-phenyl-alaninol by BOC-L-2-pyridylalaninol (step 1g). (R)-1-(3-chloro-phenyl)-ethylamine was used as the amine component for the last step:

| Example | | |
|---|---|---|
| 2.11 | 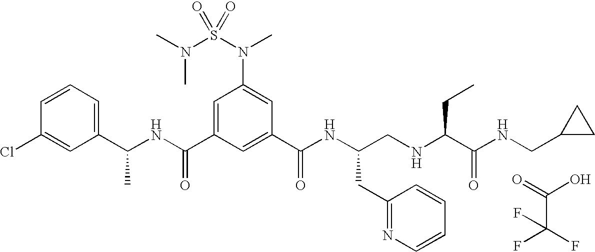 | RT (HPLC-MS) = 2.69 min. ES-MS (M + H)⁺ = 712/714 (Cl) |

### Example 3

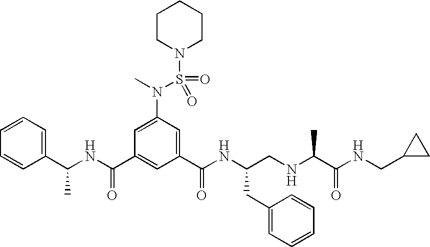

### a) Preparation of 3-a:

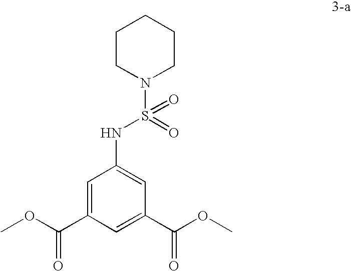

3-a was obtained analogously to 2-a, using piperidylsulphonyl chloride instead of dimethylaminosulphonyl chloride.

RT (HPLC-MS)=3.13 min

(M+H)⁺ (HPLC-MS)=356

### b) Preparation of 3-b:

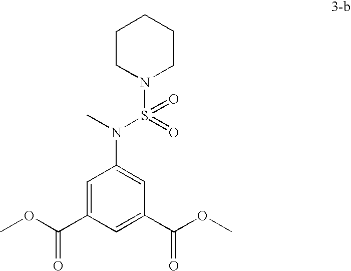

3-b was obtained analogously to 2-b from 3-a.

RT (HPLC-MS)=3.34 min

(M+H)⁺ (HPLC-MS)=370

### c) Preparation of 3-c:

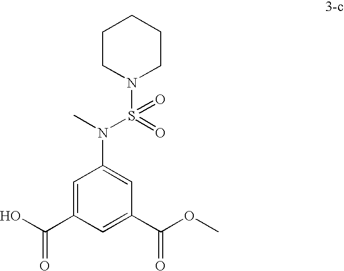

3-c was obtained analogously to 2-c from 3-b.

RT (HPLC-MS)=2.91 min

(M+H)⁺ (HPLC-MS)=356

### d) Preparation of 3-d:

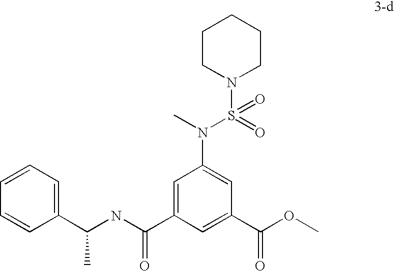

1.45 g (3.25 mmol) 3-c in 30 ml dichloromethane were combined with 1.04 g (3.25 mmol) TBTU and 1.67 ml (9.75 mmol) DIPEA, then 0.42 ml (3.25 mmol) (R)-1-phenyl-ethylamine was added and the mixture was stirred for 1 hour at ambient temperature. The reaction solution was extracted with 20% KHCO₃ solution and water. The organic phases were separated using a phase separation cartridge and evaporated to dryness i. vac. The residue was purified by chromatography on silica gel with the eluant (ethyl acetate/heptane 9:1). This yielded 2.24 g (90%) of beige crystals 3-d.

### e) Preparation of 3-e:

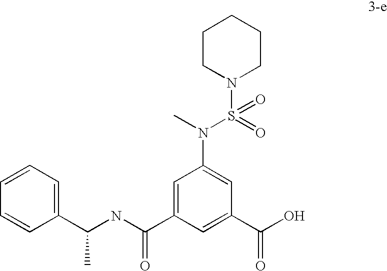

3-e was obtained analogously to 1-d from 3-d.

RT (HPLC-MS)=3.05 min

(M+H)⁺ (HPLC-MS)=445

### f) Preparation of 3-f:

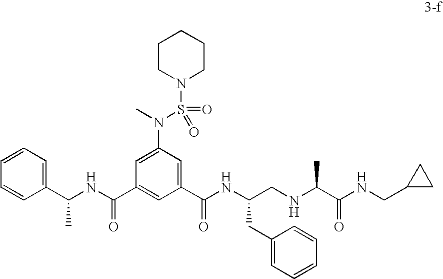

3-f was obtained analogously to 1-j from 3-e and 1-i.

RT (HPLC-MS)=3.02 min

(M+H)⁺ (HPLC-MS)=703

The following compounds were obtained analogously to Example 3 using the corresponding sulphonyl chlorides:

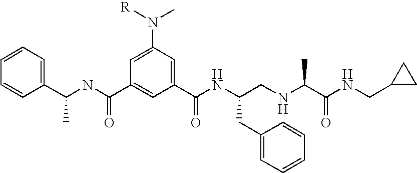

| Example | R | |
|---|---|---|
| 3.1 | 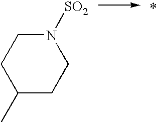 | RT (HPLC-MS) = 3.08 min ES-MS (M + H)⁺ = 717 |
| 3.2 | 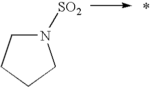 | RT (HPLC-MS) = 2.94 min ES-MS (M + H)⁺ = 689 |

### Example 4

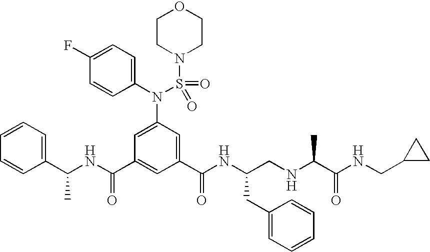

### a) Preparation of 4-a:

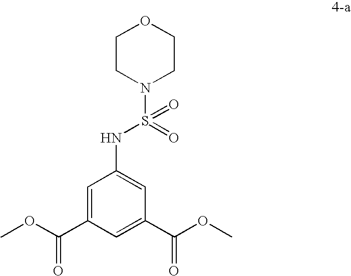

4-a was obtained analogously to 2-a, using morpholinylsulphonyl chloride instead of dimethylaminosulphonyl chloride.

RT (HPLC-MS)=3.58 min

(M+H)⁺ (HPLC-MS)=359

### b) Preparation of 4-b:

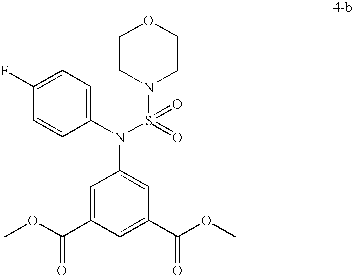

1 g (2.79 mmol) 4-a and 780 mg (5.56 mmol) 4-fluorophenylboric acid in 20 ml dichloromethane were combined with 590 mg (3.25 mmol) copper-(II)-acetate, 800 μl (5.77 mmol) triethylamine and 500 mg molecular sieve 4 Å. The reaction solution was stirred overnight at ambient temperature and filtered through silica gel. The filtrate was washed first with 2N HCl and then with sat. NaHCO₃ solution. The organic phases were separated through phase separation cartridges and evaporated to dryness. The residue was purified by HPLC. This yielded 200 mg (16%) 4-b.

RT (HPLC-MS)=2.75 min

(M+H)⁺ (HPLC-MS)=453

### c) Preparation of 4-c:

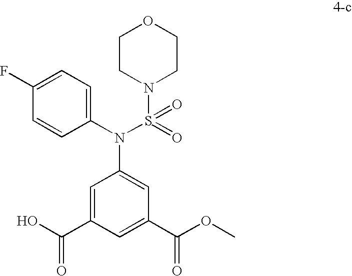

4-c was obtained analogously to 2-c from 4-b.

RT (HPLC-MS)=2.98 min

(M+H)⁺ (HPLC-MS)=439

### d) Preparation of 4-d:

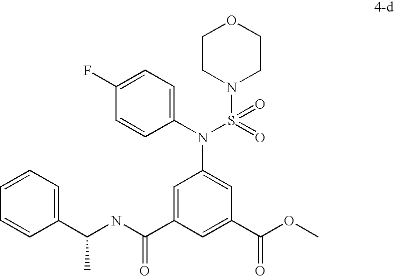

4-d was obtained analogously to 3-d from 4-c.

RT (HPLC-MS)=3.30 min

(M+H)⁺ (HPLC-MS)=543

### e) Preparation of 4-e:

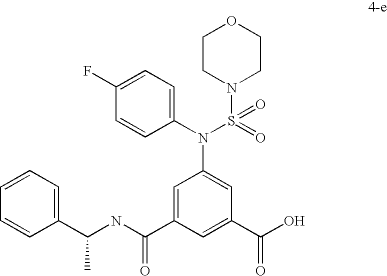

4-e was obtained analogously to 1-d from 4-d.

RT (HPLC-MS)=3.11 min

(M+H)⁺ (HPLC-MS)=528

### f) Preparation of 4-f:

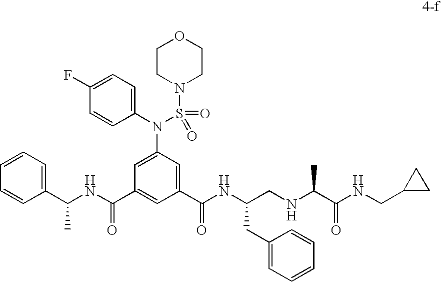

4-f was obtained analogously to 1-j from 4-e and 1-i.

ES-MS (M+H)⁺=785

### Example 5

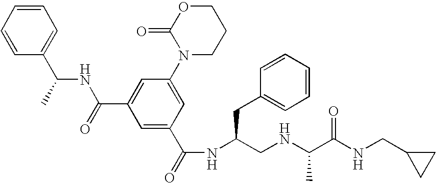

Example 5 was prepared analogously to Example 1 from 5-c and the corresponding precursors.

ES-MS (M+H)⁺=626

RT (HPLC-MS): 2.66 min

### a) Preparation of 5-a:

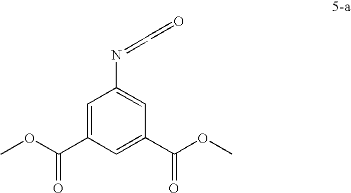

10.46 g (50 mmol) dimethyl 5-amino-isophthalate were dissolved in 200 ml of toluene and combined with 7.3 ml (60 mmol) diphosgene. The reaction solution was refluxed for 1 h. Then the reaction solution was evaporated down i. vac., twice mixed with toluene and distilled off again. The residue (10.6 g) was used without purification for preparing 5-b.

### b) Preparation of 5-b:

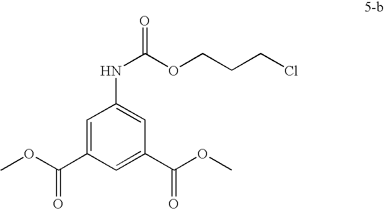

10.6 g (45 mmol) 5-a were dissolved in 450 ml of toluene and combined with 3.88 ml (45 mmol) 3-chloro-1-propanol. The reaction solution was heated to 75° C. for 1 h. Then the reaction solution was evaporated down i. vac. The residue was purified by chromatography on silica gel with the eluant (ethyl acetate/heptane 7:3). This yielded 8.5 g of 5-b (57%)

ES-MS (M+H)⁺=330

### c) Preparation of 5-c:

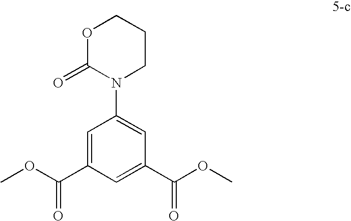

8.49 g (25.8 mmol) 5-b were dissolved in 140 ml acetonitrile, combined with 4.27 g (30.9 mmol) potassium carbonate and refluxed for 2 h. Then any insoluble ingredients were filtered off, the reaction solution was evaporated down i. vac. and stirred with ether. The resulting crystals were filtered off and washed with ether. This yielded 6.5 g of 5-c (77%)

ES-MS (M+H)⁺=294

The following compounds were prepared analogously to Example 5 using the corresponding educts:

| Example | | |
|---|---|---|
| 5.1 | 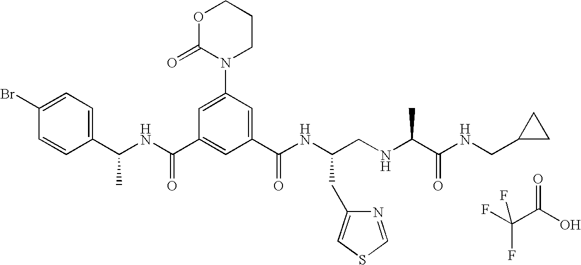 | RT (HPLC-MS) = 2.62 min. ES-MS (M + H)⁺ = 711/713 (Br) |
| 5.2 | 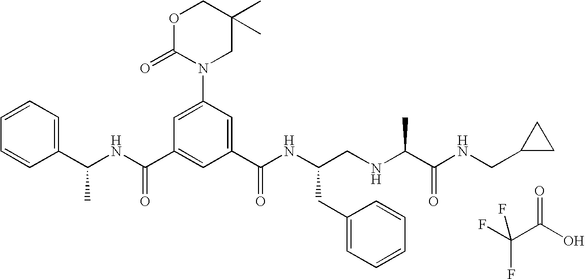 | RT (HPLC-MS) = 2.79 min. ES-MS (M + H)⁺ = 654 |
| 5.3 | 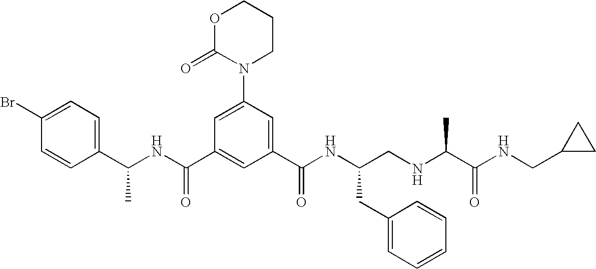 | RT (HPLC-MS) = 2.79 min. ES-MS (M + H)⁺ = 704/706 (Br) |
| 5.4 | 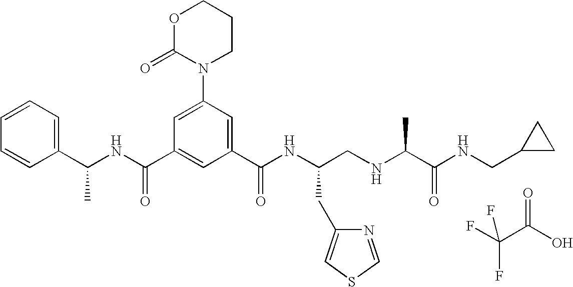 | RT (HPLC-MS) = 2.40 min. ES-MS (M + H)⁺ = 633 |
| 5.5 | 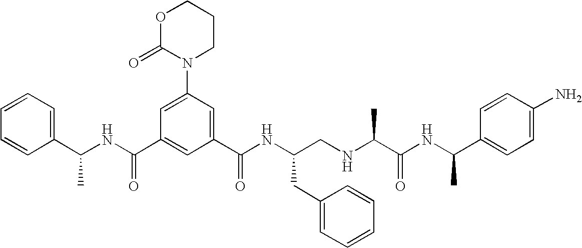 | RT (HPLC-MS) = 2.38 min. ES-MS (M + H)⁺ = 691 |

The following are examples of preparation forms in which the term “active substance” denotes one or more compounds according to the invention including the salts thereof.

In the case of one of the combinations with one or more additional active substances the term “active substance” also includes the additional active substances.

### Example A

Tablets containing 100 mg of active substance

Composition
1 tablet contains:

| | | |
|---|---|---|
| active substance | 100.0 | mg |
| lactose | 80.0 | mg |
| corn starch | 34.0 | mg |
| polyvinylpyrrolidone | 4.0 | mg |
| magnesium stearate | 2.0 | mg |
| | 220.0 | mg |

### Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.
Weight of tablet: 220 mg
Diameter: 10 mm, biplanar, facetted on both sides and notched on one side.

### Example B

Tablets containing 150 mg of active substance

Composition
1 tablet contains:

| | | |
|---|---|---|
| active substance | 150.0 | mg |
| powdered lactose | 89.0 | mg |
| corn starch | 40.0 | mg |
| colloidal silica | 10.0 | mg |
| polyvinylpyrrolidone | 10.0 | mg |
| magnesium stearate | 1.0 | mg |
| | 300.0 | mg |

### Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.

| | |
|---|---|
| Weight of tablet: | 300 mg |
| die: | 10 mm, flat |

### Example C

Hard gelatine capsules containing 150 mg of active substance

Composition
1 capsule contains:

| | | | |
|---|---|---|---|
| active substance | | 150.0 | mg |
| corn starch (dried | approx. | 180.0 | mg |
| lactose (powdered) | approx. | 87.0 | mg |
| magnesium stearate | | 3.0 | mg |
| | approx. | 420.0 | mg |

### Preparation:

The active substance is mixed with the excipients, passed through a screen with a mesh size of 0.75 mm and homogeneously mixed using a suitable apparatus. The finished mixture is packed into size 1 hard gelatine capsules.
Capsule filling: approx. 320 mg
Capsule shell: size 1 hard gelatine capsule.

### Example D

Suppositories containing 150 mg of active substance

Composition
1 suppository contains:

| | | |
|---|---|---|
| active substance | 150.0 | mg |
| polyethyleneglycol 1500 | 550.0 | mg |
| polyethyleneglycol 6000 | 460.0 | mg |
| polyoxyethylene sorbitan monostearate | 840.0 | mg |
| | 2,000.0 | mg |

### Preparation:

After the suppository mass has been melted the active substance is homogeneously distributed therein and the melt is poured into chilled moulds.

### Example E

Ampoules containing 10 mg active substance

Composition

| | | | |
|---|---|---|---|
| active substance | | 10.0 | mg |
| 0.01 N hydrochloric acid | q.s. | | |
| double-distilled water | ad | 2.0 | ml |

### Preparation:

The active substance is dissolved in the necessary amount of 0.01 N HCl, made isotonic with common salt, filtered sterile and transferred into 2 ml ampoules.

### Example F

Ampoules containing 10 mg active substance

Composition

| | | | |
|---|---|---|---|
| active substance | | 50.0 | mg |
| 0.01 N hydrochloric acid | q.s. | | |
| double-distilled water | ad | 10.0 | ml |

### Preparation:

The active substance is dissolved in the necessary amount of 0.01 N HCl, made isotonic with common salt, filtered sterile and transferred into 10 ml ampoules.

1. Compounds of general formula (I) 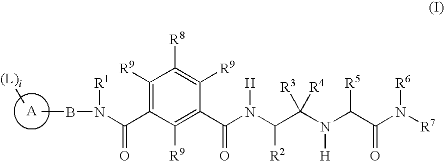
wherein
A denotes aryl or heteroaryl,
wherein the group A, besides the groups L, may optionally be substituted by one or more fluorine atoms,
L in each case independently of one another denote hydrogen, fluorine, chlorine, bromine, iodine, hydroxy, carboxy, formyl, cyano, nitro, F₃C, HF₂C, FH₂C, C₁₋₆-alkyl, C₂₋₆-alkenyl, C₂₋₆-alkynyl, C₁₋₆-alkyl-S, C₁₋₆-alkyl-S—C₁₋₃-alkyl, C₃₋₇-cycloalkyl, C₃₋₇-cycloalkyl-C₁₋₆-alkyl, C₃₋₇-cycloalkyl-C₂₋₆-alkenyl, C₃₋₇-cycloalkyl-C₂₋₆-alkynyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, heterocyclyl, heterocyclyl-C₁₋₆-alkyl, heterocyclyl-C₂₋₆-alkenyl, heterocyclyl-C₂₋₆-alkynyl, aryl, aryl-C₁₋₆-alkyl, aryl-C₂₋₆-alkenyl, aryl-C₂₋₆-alkynyl, aryl-C₃₋₇-cycloalkyl, heteroaryl, heteroaryl-C₁₋₆-alkyl, heteroaryl-C₂₋₆-alkenyl, heteroaryl-C₂₋₆-alkynyl, heteroaryl-C₃₋₇-cycloalkyl, R¹³—O, R¹³—O—C₁₋₃-alkyl, (R¹²)₂N, (R¹²)₂N—CO, R¹²—CO—(R¹²)N, (R¹²)₂N—CO—(R¹²)N, R¹²—SO₂(R¹²)N, (R¹²)₂N—SO₂ or C₁₋₆-alkyl-SO₂₅ wherein the above mentioned groups may optionally be substituted independently of one another by one or more groups selected from among fluorine, chlorine, bromine, iodine, hydroxy, oxo, carboxy, formyl, cyano, nitro, F₃C, HF₂C, FH₂C, hydroxy-C₁₋₆-alkyl, C₁₋₃-alkyl, C₁₋₆-alkoxy, (R¹²)₂N, (R¹²)₂N—C₁₋₃-alkyl, (R¹²)₂N—CO— and HOSO₂—,
i denotes 0, 1, 2 or 3,
B denotes a C₁₋₄-alkylene bridge,
while the C₁₋₄-alkylene bridge may optionally be substituted by one or more groups selected from among fluorine, chlorine, bromine, iodine, hydroxy, oxo, carboxy, cyano, nitro, F₃C, HF₂C, FH₂C, C₁₋₄-alkyl, C₁₋₆-alkyl-S—C₁₋₃-alkyl, C₃₋₇-cycloalkyl, heterocyclyl, heterocyclyl-C₁₋₃-alkyl, aryl, aryl-C₁₋₃-alkyl, aryl-C₃₋₇-cycloalkyl, heteroaryl, heteroaryl-C₁₋₃-alkyl, heteroaryl-C₃₋₇-cycloalkyl, R¹³—O, (R¹²)₂N—SO₂, (R¹²)₂N, (R¹²)₂N—C₁₋₃-alkyl, (R¹²)₂N—CO, R¹²—SO₂, R¹²—CO—(R¹²)N, R¹²—SO₂(R¹²)N, (R¹²)₂N—SO₂, R¹²—CO— and R¹²—SO—, and wherein two C₁₋₄-alkyl groups bound to the same carbon atom of the C₁₋₄-alkylene bridge may be joined together, forming a C₃₋₇-cycloalkyl group, and
wherein the above mentioned C₁₋₄-alkyl groups and the C₃₋₇-cycloalkyl group formed from the C₁₋₄-alkyl groups may optionally be substituted independently of one another by one or more groups selected from among fluorine, chlorine, bromine, iodine, hydroxy, oxo, carboxy, formyl, cyano, nitro, F₃C, C₁₋₃-alkyl, C₁₋₃-alkoxy, R¹³—O—C₁₋₃-alkyl, R¹²—CO(R¹²)N, R¹²—SO₂(R¹²)N, (R¹²)₂N, (R¹²)₂N—C₁₋₃-alkyl, (R¹²)₂N—CO, (R¹²)₂N—SO₂— and HOSO₂—,
R¹ denotes hydrogen, C₁₋₆-alkyl, C₂₋₆-alkenyl, C₂₋₆-alkynyl, C₃₋₇-cycloalkyl, C₃₋₇-cycloalkyl-C₁₋₆-alkyl, C₃₋₇-cycloalkyl-C₂₋₆-alkenyl, C₃₋₇-cycloalkyl-C₂₋₆-alkynyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, heterocyclyl, heterocyclyl-C₁₋₆-alkyl, heterocyclyl-C₂₋₆-alkenyl, heterocyclyl-C₂₋₆-alkynyl, aryl, aryl-C₁₋₆-alkyl, aryl-C₂₋₆-alkenyl, aryl-C₂₋₆-alkynyl, aryl-C₃₋₇-cycloalkyl, heteroaryl, heteroaryl-C₁₋₆-alkyl, heteroaryl-C₂₋₆-alkenyl, heteroaryl-C₂₋₆-alkynyl or heteroaryl-C₃₋₇-cycloalkyl,
wherein the above mentioned groups may optionally be substituted independently of one another by one or more groups selected from among fluorine, chlorine, bromine, iodine, hydroxy, oxo, carboxy, formyl, cyano, nitro, F₃C, C₁₋₃-alkyl, C₁₋₃-alkoxy, hydroxy-C₁₋₆-alkyl, (R¹²)₂N, (R¹²)₂N—C₁₋₃-alkyl, (R¹²)₂N—CO, (R¹²)₂N—SO₂, R¹²—CO—(R¹²)N, R¹²—SO₂(R¹²)N— and HOSO₂—,
R² denotes C₁₋₆-alkyl, C₂₋₆-alkenyl, C₂₋₆-alkynyl, C₁₋₆-alkoxy-C₁₋₃-alkyl, C₁₋₆-alkyl-S—C₁₋₃-alkyl, C₃₋₇-cycloalkyl, C₃₋₇-cycloalkyl-C₁₋₃-alkyl, C₃₋₇-cycloalkyl-C₂₋₃-alkenyl, C₃₋₇-cycloalkyl-C₂₋₃-alkynyl, C₃₋₇-cycloalkenyl, C₃₋₇-cycloalkenyl-C₁₋₃-alkyl, C₃₋₇-cycloalkenyl-C₂₋₃-alkenyl, C₃₋₇-cycloalkenyl-C₂₋₃-alkynyl, heterocyclyl, heterocyclyl-C₁₋₃-alkyl, heterocyclyl-C₂₋₃-alkenyl, heterocyclyl-C₂₋₃-alkynyl, aryl, aryl-C₁₋₃-alkyl, aryl-C₂₋₃-alkenyl, aryl-C₂₋₃-alkynyl, aryl-C₃₋₇-cycloalkyl, heteroaryl, heteroaryl-C₁₋₃-alkyl, heteroaryl-C₂₋₃-alkenyl, heteroaryl-C₂₋₃-alkynyl or heteroaryl-C₃₋₇-cycloalkyl,
wherein the above mentioned groups may optionally be substituted independently of one another by one or more groups selected from among fluorine, chlorine, bromine, iodine, F₃C, F₃C, HF₂C, FH₂C— hydroxy, oxo, carboxy, formyl, cyano, nitro, (R¹²)₂N, (R¹²)₂N—C₁₋₃-alkyl, HOSO₂, C₁₋₃-alkyl, C₁₋₆-alkyl-S—C₁₋₃-alkyl, (R¹²)₂N—SO₂, R¹²—CO—(R¹²)N, R¹²—SO₂(R¹²)N, (R¹²)₂N—C₁₋₃-alkyl, (R¹²)₂N—CO, R¹³—O— and R¹³—O—C₁₋₃-alkyl-,
R³, R⁴ in each case independently of one another denote hydrogen, C₁₋₆-alkyl, fluorine, F₃C, HF₂C or FH₂C,
R⁵ denotes hydrogen, C₁₋₆-alkyl, C₂₋₆-alkenyl, C₂₋₆-alkynyl, C₃₋₇-cycloalkyl, C₃₋₇-cycloalkyl-C₁₋₄-alkyl, C₃₋₇-cycloalkyl-C₂₋₄-alkenyl, C₃₋₇-cycloalkyl-C₂₋₄-alkynyl, C₃₋₇-cycloalkenyl, C₃₋₇-cycloalkenyl-C₁₋₄-alkyl, C₃₋₇-cycloalkenyl-C₂₋₄-alkenyl, C₃₋₇-cycloalkenyl-C₂₋₄-alkynyl, heterocyclyl, heterocyclyl-C₁₋₄-alkyl, heterocyclyl-C₂₋₄-alkenyl, heterocyclyl-C₂₋₄-alkynyl, aryl, aryl-C₁₋₄-alkyl, aryl-C₂₋₄-alkenyl, aryl-C₂₋₄-alkynyl, aryl-C₃₋₇-cycloalkyl, heteroaryl, heteroaryl-C₁₋₄-alkyl, heteroaryl-C₂₋₄-alkenyl, heteroaryl-C₂₋₄-alkynyl or heteroaryl-C₃₋₇-cycloalkyl, wherein the above mentioned groups may optionally be substituted independently of one another by one or more groups selected from among fluorine, chlorine, bromine, iodine, hydroxy, oxo, carboxy, formyl, cyano, nitro, C₁₋₃-alkyl, C₁₋₆-alkoxy, C₁₋₃-alkyl-S, aryl, heteroaryl, heteroaryl-C₁₋₃-alkyl, aryl-C₁₋₆-alkyl, R¹²—CO—(R¹²)N, R¹²—SO₂(R¹²)N—(R¹²)₂N—SO₂, (R¹²)₂N, (R¹²)₂N—C₁₋₃-alkyl, (R¹²)₂N—CO— and HOSO₂—,
R⁶ denotes hydrogen, C₁₋₆-alkyl, C₂₋₆-alkenyl, C₂₋₆-alkynyl, C₃₋₇-cycloalkyl, C₃₋₇-cycloalkyl-C₁₋₆-alkyl, C₃₋₇-cycloalkyl-C₂₋₆-alkenyl, C₃₋₇-cycloalkyl-C₂₋₆-alkynyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl,
heterocyclyl, heterocyclyl-C₁₋₆-alkyl, heterocyclyl-C₂₋₆-alkenyl, heterocyclyl-C₂₋₆-alkynyl, aryl, aryl-C₁₋₆-alkyl, aryl-C₂₋₆-alkenyl, aryl-C₂₋₆-alkynyl, aryl-C₃₋₇-cycloalkyl, heteroaryl, heteroaryl-C₁₋₆-alkyl, heteroaryl-C₂₋₆-alkenyl, heteroaryl-C₂₋₆-alkynyl or heteroaryl-C₃₋₇-cycloalkyl,
wherein the above mentioned groups may optionally be substituted independently of one another by one or more groups selected from among fluorine, chlorine, bromine, iodine, hydroxy, oxo, carboxy, formyl, cyano, nitro, C₁₋₃-alkyl, heterocyclyl, heterocyclyl-C₁₋₃-alkyl, R¹³—O, R¹³—O—C₁₋₃-alkyl, aryl, heteroaryl, heteroaryl-C₁₋₃-alkyl, aryl-C₁₋₆-alkyl, (R¹²)₂N, (R¹²)₂N—C₁₋₃-alkyl, (R¹²)₂N—CO, (R¹²)₂N—CO—N(R¹²), (R¹²)₂N—SO₂— and HOSO₂—,
R⁷ denotes hydrogen, C₁₋₆-alkyl, C₂₋₆-alkenyl, C₂₋₆-alkynyl, C₁₋₆-alkoxy-C₁₋₃-alkyl, C₃₋₇-cycloalkyl, C₃₋₇-cycloalkyl-C₁₋₃-alkyl, heterocyclyl-C₁₋₃-alkyl, aryl, aryl-C₁₋₃-alkyl, heteroaryl or heteroaryl-C₁₋₃-alkyl,
wherein the above mentioned groups may optionally be substituted independently of one another by one or more groups selected from among fluorine, chlorine, bromine, iodine, cyano, hydroxy, C₁₋₃-alkyl, C₁₋₆-alkoxy and (R¹²)₂N,
R⁸ denotes C₁₋₆-alkyl, C₂₋₆-alkenyl, C₂₋₆-alkynyl, C₃₋₇-cycloalkyl, C₃₋₇-cycloalkyl-C₁₋₃-alkyl, heterocyclyl, heterocyclyl-C₁₋₃-alkyl, C₃₋₇-cycloalkyl-C₂₋₄-alkenyl, heterocyclyl-C₂₋₄-alkenyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, aryl-C₁₋₃-alkyl, heteroaryl, heteroaryl-C₁₋₃-alkyl, aryl-C₂₋₃-alkenyl, heteroaryl-C₂₋₃-alkenyl, aryl-C₂₋₃-alkynyl, aryl-C₃₋₇-cycloalkyl, heteroaryl-C₂₋₃-alkynyl, heteroaryl-C₃₋₇-cycloalkyl, C₃₋₇-cycloalkyl-C₂₋₄-alkynyl, heterocyclyl-C₂₋₄-alkynyl, R¹³—O, R¹³—O—C₁₋₃-alkyl, R¹⁰—SO₂—(R¹¹)N or R¹⁰—CO—(R¹¹)N,
wherein the C₁₋₆-alkyl group and the C₃₋₇-cycloalkyl group may optionally be substituted independently of one another by one or more groups selected from among C₁₋₆-alkyl, fluorine, chlorine, bromine, hydroxy, oxo, carboxy, formyl, nitro, C₂₋₇-alkenyl, C₂₋₇-alkynyl, C₁₋₆-alkyl-S, C₁₋₆-alkyl-S—C₁₋₃-alkyl, C₃₋₆-cycloalkyl, C₃₋₇-cycloalkyl-C₁₋₆-alkyl, aryl, aryl-C₁₋₆-alkyl, heterocyclyl, heterocyclyl-C₁₋₆-alkyl, heteroaryl, heteroaryl-C₁₋₆-alkyl, R¹³—O, R¹³—O—CO, R¹³—CO, R¹³—O—CO—(R¹²)N, (R¹²)₂N—CO—O, R¹³—O—C₁₋₃-alkyl, (R¹²)₂N, (R¹²)₂N—CO, R¹²—CO—(R¹²)N, (R¹²)₂N—CO—(R¹²)N, (R¹²)₂N—SO₂, (R¹²)₂N—SO₂—(R¹²)N, R¹²—SO₂, F₃C, HF₂C, FH₂C, F₃C—O, HF₂C—O, FH₂C—O— or R¹²—SO₂—(R¹²)N—, and wherein the above mentioned C₂₋₆-alkenyl, C₂₋₆-alkynyl, C₃₋₇-cycloalkyl-C₁₋₃-alkyl, heterocyclyl, heterocyclyl-C₁₋₃-alkyl, C₃₋₇-cycloalkyl-C₂₋₄-alkenyl, heterocyclyl-C₂₋₄-alkenyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, aryl-C₁₋₃-alkyl, heteroaryl, heteroaryl-C₁₋₃-alkyl, aryl-C₂₋₃-alkenyl, heteroaryl-C₂₋₃-alkenyl, aryl-C₂₋₃-alkynyl, aryl-C₃₋₇-cycloalkyl, heteroaryl-C₂₋₃-alkynyl, heteroaryl-C₃₋₇-cycloalkyl, C₃₋₇-cycloalkyl-C₂₋₄-alkynyl and heterocyclyl-C₂₋₄-alkynyl groups may optionally be substituted independently of one another by one or more groups selected from among C₁₋₆-alkyl, fluorine, chlorine, bromine, hydroxy, oxo, carboxy, formyl, cyano, nitro, C₂₋₆-alkenyl, C₂₋₆-alkynyl, C₁₋₆-alkyl-S, C₁₋₆-alkyl-S—C₁₋₃-alkyl, C₃₋₇-cycloalkyl, C₃₋₇-cycloalkyl-C₁₋₆-alkyl, aryl, aryl-C₁₋₆-alkyl, heterocyclyl, heterocyclyl-C₁₋₆-alkyl, heteroaryl, heteroaryl-C₁₋₆-alkyl, R¹³—O, R¹³—O—CO, R¹³—CO, R¹³—CO, R¹³—O—CO—(R¹²)N, (R¹²)₂N—CO—O, R¹³—O—C₁₋₃-alkyl, (R¹²)₂N, (R¹²)₂N—CO, R¹²—CO—(R¹²)N, (R¹²)₂N—CO—(R¹²)N, (R¹²)₂N—SO₂, (R¹²)₂N—SO₂—(R¹²)N, R¹²—SO₂, F₃C, HF₂C, FH₂C, F₃C—O, HF₂C—O, FH₂C—O— or R¹²—SO₂—(R¹²)N,
R⁹ in each case independently of one another denote hydrogen, fluorine, chlorine, bromine, iodine, C₁₋₃-alkyl, R¹³—O or (R¹²)₂N,
wherein the above mentioned C₁₋₃-alkyl group may optionally be substituted by one or more fluorine atoms,
R¹⁰ denotes C₂₋₆-alkenyl, C₂₋₆-alkynyl, C₃₋₇-cycloalkyl-C₂₋₄-alkenyl, aryl-C₂₋₃-alkenyl, heteroaryl-C₂₋₃-alkenyl, heterocyclyl-C₂₋₄-alkenyl, aryl-C₂₋₃-alkynyl, C₃₋₇-cycloalkyl-C₂₋₄-alkynyl, heterocyclyl-C₂₋₄-alkynyl-heteroaryl-C₂₋₃-alkynyl, C₃₋₇-cycloalkyl, heterocyclyl, heterocyclyl-C₁₋₃-alkyl, C₃₋₇-cycloalkenyl, C₃₋₇-cycloalkenyl-C₁₋₆-alkyl, C₃₋₇-cycloalkenyl-C₂₋₆-alkenyl, C₃₋₇-cycloalkenyl-C₂₋₆-alkynyl, aryl-C₂₋₃-alkyl, aryl-C₃₋₇-cycloalkyl, heteroaryl, heteroaryl-C₁₋₃-alkyl, heteroaryl-C₃₋₇-cycloalkyl or (R¹²)₂N,
wherein the above mentioned groups may optionally be substituted independently of one another by one or more groups selected from among fluorine, chlorine, bromine, hydroxy, oxo, carboxy, formyl, cyano, nitro, C₁₋₃-alkyl, heterocyclyl, heterocyclyl-C₁₋₃-alkyl, R¹³—O, R¹³—O—C₁₋₃-alkyl, R¹²—CO(R¹²)N, R¹²—SO₂(R¹²)N, (R¹²)₂N, (R¹²)₂N—C₁₋₃-alkyl and (R¹²)₂N—CO,
R¹¹ denotes C₁₋₆-alkyl, C₂₋₆-alkenyl, C₂₋₆-alkynyl, C₃₋₇-cycloalkyl-C₁₋₃-alkyl, aryl, aryl-C₂₋₃-alkyl, heterocyclyl, heterocyclyl-C₁₋₃-alkyl, heteroaryl or heteroaryl-C₁₋₃-alkyl,
wherein the above mentioned groups may optionally be substituted independently of one another by one or more groups selected from among fluorine, chlorine, bromine, hydroxy, oxo, carboxy, formyl, cyano, nitro, C₁₋₃-alkyl, heterocyclyl, heterocyclyl-C₁₋₃-alkyl, R¹³—O, R¹³—O—C₁₋₃-alkyl, (R¹²)₂N, (R¹²)₂N—C₁₋₃-alkyl- and R¹²CO—,
or
R¹⁰ and R¹¹ together form a C₂₋₆-alkylene bridge, so that a heterocyclic ring is formed with the inclusion of the nitrogen atom linked to R¹¹ and the SO₂— or CO— group linked to R¹⁰,
wherein one or two —CH₂ groups of the C₂₋₆-alkylene bridge may be replaced independently of one another by O, S or —N(R¹²)— such that in each case two O, S or N atoms or an O and an S atom are not directly connected to one another, and
wherein the C atoms of the above mentioned C₂₋₆-alkylene bridge may optionally be substituted by one or more groups selected from among fluorine, chlorine, bromine, hydroxy, carboxy, formyl, cyano, F₃C, C₁₋₆-alkyl, C₁₋₆-alkoxy, oxo and nitro,
R¹² in each case independently of one another denote hydrogen, C₁₋₆-alkyl, C₁₋₆-alkoxy-C₁₋₃-alkyl, C₃₋₆-cycloalkyl, C₃₋₆-cycloalkyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl, wherein
two C₁₋₆-alkyl groups bound to the same nitrogen atom may together form a C₂₋₆-alkylene bridge, so that a heterocyclic ring is formed with the inclusion of the nitrogen atoms linked to the groups R¹²,
while a —CH₂ group of the C₂₋₆-alkylene bridge may be replaced by O, S or —N(R¹³)—, and
wherein the above mentioned groups and the heterocyclic ring may optionally be substituted independently of one another by one or more groups selected from among fluorine, chlorine, bromine, iodine, hydroxy, oxo, carboxy, formyl, cyano, nitro, C₁₋₃-alkyl, hydroxy-C₁₋₃-alkyl, C₁₋₃-alkoxy, (R¹³)₂N—CO or (R¹³)₂N—, and
R¹³ in each case independently of one another denote hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl, wherein the above mentioned groups may optionally be substituted independently of one another by one or more groups selected from among fluorine, chlorine, bromine, iodine, hydroxy, oxo, carboxy, formyl, cyano, nitro, C₁₋₃-alkyl- and C₁₋₃-alkoxy-,
the pharmacologically acceptable salts, diastereomers, enantiomers, racemates, hydrates and solvates thereof.

2. Compounds according to claim 1, characterised in that
A denotes phenyl or a 5- or 6-membered aromatic heteroaryl group which contains 1, 2 or 3 heteroatoms selected from N, O and S.

3. Compounds according to claim 1, characterised in that the group 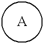
is selected from among

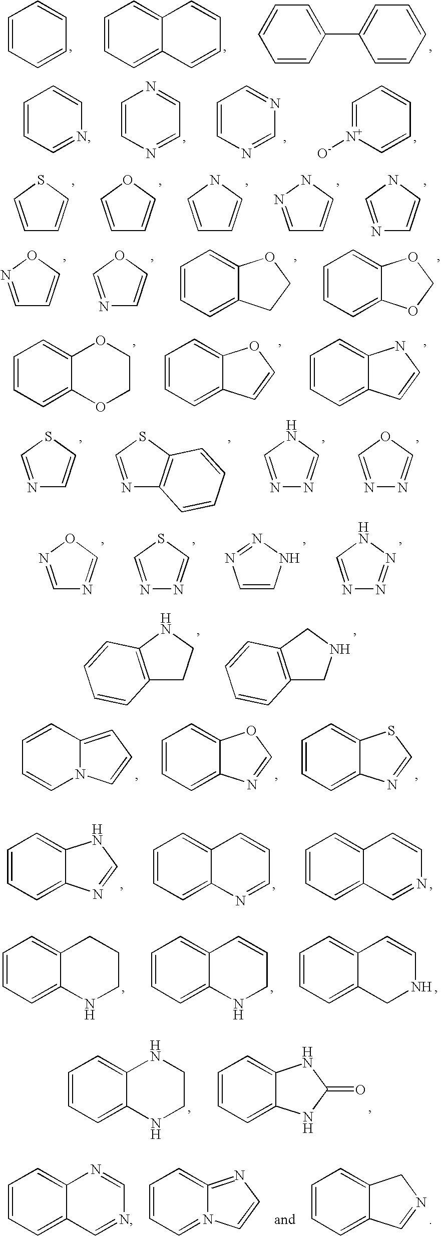

4. Compounds according to claim 1, characterised in that
A denotes phenyl, thienyl, thiazolyl, pyrazolyl or pyridyl.

5. Compounds according to claim 1, characterised in that
L in each case independently of one another denotes hydrogen, fluorine, chlorine, bromine, iodine, hydroxy, carboxy, cyano, nitro, F₃C, HF₂C, FH₂C, C₁₋₆-alkyl, C₂₋₆-alkenyl, C₂₋₆-alkynyl, C₃₋₇-cycloalkyl, C₃₋₇-cycloalkyl-C₁₋₃-alkyl, aryl, aryl-C₁₋₃-alkyl, heterocyclyl, heterocyclyl-C₁₋₃-alkyl, heteroaryl, heteroaryl-C₁₋₃-alkyl, R¹³—O, R¹³—O—C₁₋₃-alkyl, (R¹²)₂N, (R¹²)₂N—CO, R¹²—CO—(R¹²)N, (R¹²)₂N—CO—(R¹²)N, (R¹²)₂N—SO₂, R¹²—SO₂—(R¹²)N or C₁₋₃-alkyl-SO₂,
wherein the above mentioned groups may optionally be substituted independently of one another by one or more groups selected from among fluorine, chlorine, bromine, hydroxy, oxo, carboxy, cyano, nitro, F₃C, HF₂C, FH₂C, hydroxy-C₁₋₃-alkyl, C₁₋₃-alkyl, C₁₋₃-alkoxy, (R¹²)₂N, (R¹²)₂N—C₁₋₃-alkyl- and (R¹²)₂N—CO—, and
i denotes 0, 1 or 2.

6. Compounds according to claim 1, characterised in that
L in each case independently of one another denotes hydrogen, fluorine, chlorine, bromine, cyano, hydroxy, C₁₋₆-alkyl, C₁₋₆-alkoxy, C₃₋₇-cycloalkyl, C₃₋₇-cycloalkyl-C₁₋₃-alkyl, phenyl, (R¹²)₂N, (R¹²)₂N—CO, R¹²—CO—(R¹²)N, (R¹²)₂N—CO—(R¹²)N, R¹²—SO₂—(R¹²)N or (R¹²)₂N—SO₂,
wherein the above mentioned groups may optionally be substituted by one or more fluorine atoms, and
i denotes 0, 1 or 2.

7. Compounds according to claim 1, characterised in that
L in each case independently of one another denotes hydrogen, fluorine, chlorine, bromine, hydroxy, C₁₋₄-alkyl or C₁₋₄-alkoxy,
wherein the above mentioned groups may optionally be substituted by one or more fluorine atoms, and
i denotes 0, 1 or 2.

8. Compounds according to claim 1, characterised in that
B denotes a C₁₋₄-alkylene bridge,
wherein the C₁₋₄-alkylene bridge may optionally be substituted independently of one another by one or more groups selected from among fluorine, hydroxy, carboxy, cyano, nitro, F₃C, HF₂C, FH₂C, C₁₋₄-alkyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl, heteroaryl-C₁₋₃-alkyl, R¹³—O, (R¹²)₂N—SO₂— and (R¹²)₂N—, and
wherein two C₁₋₄-alkyl groups bound to the same carbon atom of the C₁₋₄-alkylene bridge may be joined together, forming a C₃₋₇-cycloalkyl group, and
wherein the above mentioned groups and the C₃₋₇-cycloalkyl group formed from the C₁₋₄-alkyl groups may optionally be substituted independently of one another by one or more groups selected from among fluorine, chlorine, bromine, hydroxy, carboxy, cyano, F₃C, C₁₋₃-alkyl, C₁₋₃-alkoxy- and R¹³—O—C₁₋₃-alkyl.

9. Compounds according to claim 1, characterised in that
B denotes a C₁₋₄-alkylene bridge,
wherein the C₁₋₄-alkylene bridge may optionally be substituted independently of one another by one or more groups selected from among fluorine, C₁₋₄-alkyl, phenyl or benzyl, and
wherein two C₁₋₄-alkyl groups bound to the same carbon atom of the C₁₋₄-alkylene bridge may be joined together, forming a C₃₋₆-cycloalkyl group, and
wherein the above mentioned groups and the C₃₋₆-cycloalkyl group formed from the C₁₋₄-alkyl groups may optionally be substituted independently of one another by one or more groups selected from among fluorine, hydroxy and C₁₋₃-alkoxy.

10. Compounds according to claim 1, characterised in that
B denotes a C₁₋₂-alkylene bridge,
wherein the C₁₋₂-alkylene bridge may optionally be substituted by one or more C₁₋₄-alkyl groups, and
wherein two C₁₋₄-alkyl groups bound to the same carbon atom of the C₁₋₂-alkylene bridge may be joined together, forming a cyclopropyl group, and
wherein one or more hydrogen atoms of the above mentioned C₁₋₂-alkylene bridge and/or of the C₁₋₄-alkyl groups and/or of the cyclopropyl group formed therefrom may optionally be replaced by one or more fluorine atoms.

11. Compounds according to claim 1, characterised in that
B is selected from among 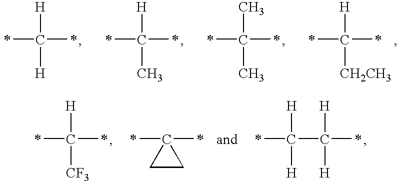
wherein one or more hydrogen atoms may optionally be replaced by fluorine.

12. Compounds according to claim 1, characterised in that the partial formula (II) 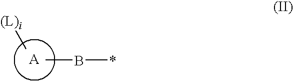
is selected from among

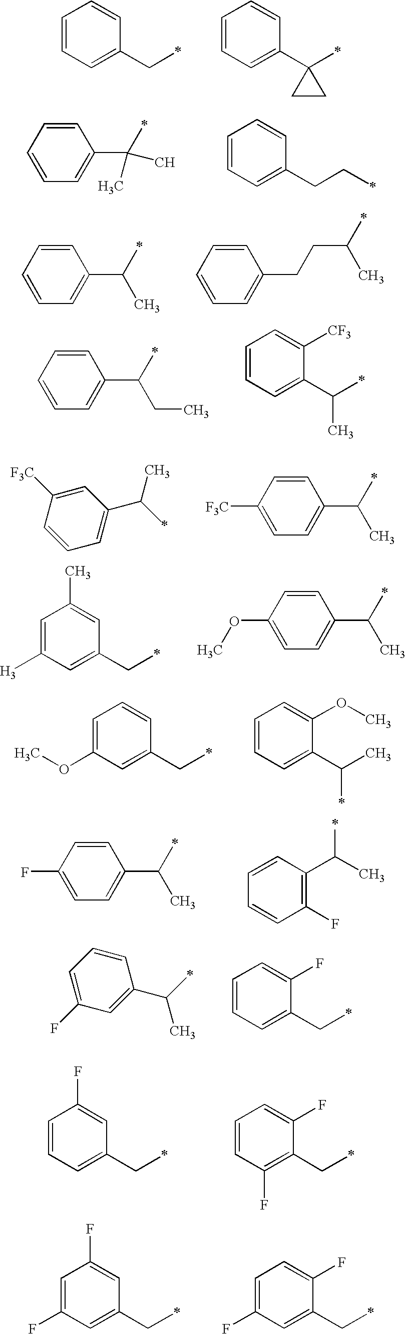

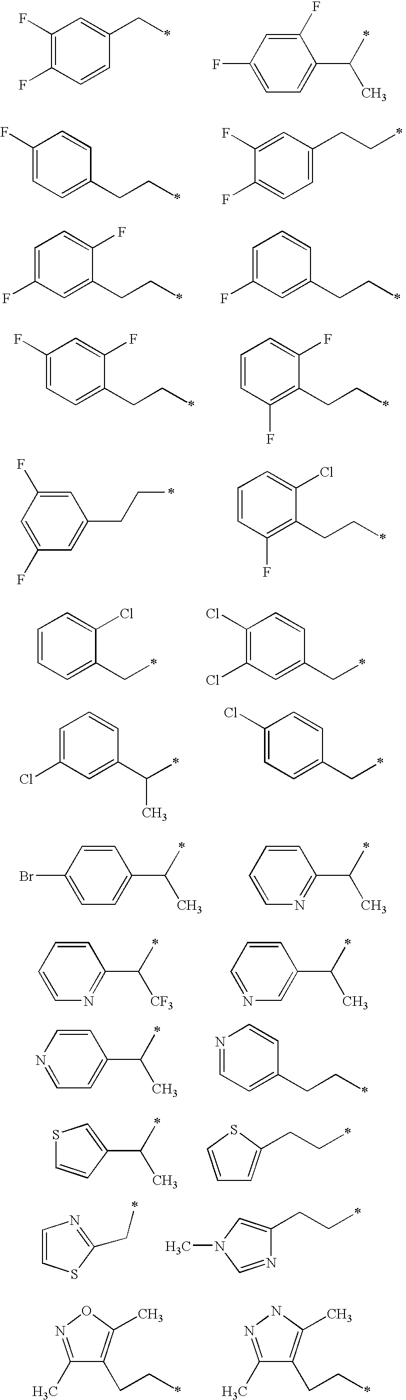

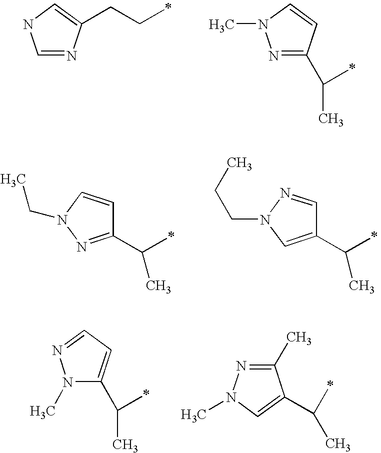

13. Compounds according to claim 1, characterised in that
R¹ denotes hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl,
wherein the above mentioned groups may optionally be substituted independently of one another by one or more groups selected from among fluorine, chlorine, bromine, hydroxy, carboxy, cyano, nitro, F₃C, C₁₋₃-alkyl, C₁₋₃-alkoxy- and hydroxy-C₁₋₃-alkyl.

14. Compounds according to claim 1, characterised in that
R¹ denotes hydrogen, C₁₋₄-alkyl, C₃₋₄-alkenyl, C₃₋₆-cycloalkyl, C₃₋₆-cycloalkyl-C₁₋₃-alkyl,
wherein the above mentioned groups may optionally be substituted independently of one another by one or more groups selected from among fluorine, hydroxy and C₁₋₃-alkoxy.

15. Compounds according to claim 1, characterised in that
R² denotes C₁₋₆-alkyl, C₂₋₆-alkenyl, C₂₋₆-alkynyl, C₁₋₆-alkoxy-C₁₋₃-alkyl, C₁₋₆-alkyl-S—C₁₋₃-alkyl, C₃₋₇-cycloalkyl, heterocyclyl, heterocyclyl-C₁₋₃-alkyl, aryl, aryl-C₁₋₃-alkyl, heteroaryl or heteroaryl-C₁₋₃-alkyl,
wherein the above mentioned groups may optionally be substituted independently of one another by one or more groups selected from among fluorine, chlorine, bromine, iodine, F₃C, HF₂C, FH₂C, hydroxy, carboxy, cyano, nitro, C₁₋₃-alkyl, (R¹²)₂N, (R¹²)₂N—SO₂, R¹²—CO—(R¹²)N, R¹²—SO₂(R¹²)N, (R¹²)₂N—C₁₋₃-alkyl, (R¹²)₂N—CO, R¹³—O— and R¹³—O—C₁₋₃-alkyl.

16. Compounds according to claim 1, characterised in that
R² denotes C₁₋₆-alkyl, C₂₋₆-alkynyl, C₃₋₆-cycloalkyl-C₁₋₃-alkyl, heterocyclyl-C₁₋₃-alkyl, phenyl, phenyl-C₁₋₃-alkyl, heteroaryl or heteroaryl-C₁₋₃-alkyl,
wherein by the above mentioned heteroaryl groups are meant 5- or 6-membered aromatic heteroaryl groups which contain 1, 2 or 3 heteroatoms selected from among N, O and S and
wherein the above mentioned groups may optionally be substituted independently of one another by one or more groups selected from among fluorine, chlorine, bromine, iodine, cyano, hydroxy, C₁₋₃-alkyl, F₃C, HF₂C, FH₂C, H₂N— and C₁₋₃-alkoxy.

17. Compounds according to claim 1, characterised in that
R² denotes n-propyl, n-butyl, 2-propynyl, 2-butynyl, cyclohexylmethyl, cyclopentylmethyl, phenylmethyl, 2-phenylethyl, pyridylmethyl, furanylmethyl, thienylmethyl or thiazolylmethyl,
wherein the above mentioned propyl, butyl-propynyl, butynyl, cyclohexylmethyl- and cyclopentylmethyl groups may optionally be substituted by one or more fluorine atoms and the pyridylmethyl, furanylmethyl, thienylmethyl or thiazolylmethyl groups may optionally be substituted independently of one another by one or more groups selected from among fluorine, chlorine, bromine, methyl, F₃C, HF₂C, FH₂C— and H₂N—.

18. Compounds according to claim 1, characterised in that
R³ denotes hydrogen, fluorine, methyl, F₃C, HF₂C or FH₂C— and
R⁴ denotes hydrogen or fluorine.

19. Compounds according to claim 1, characterised in that
R³ denotes hydrogen and
R⁴ denotes hydrogen.

20. Compounds according to claim 1, characterised in that
R⁵ denotes hydrogen, C₁₋₆-alkyl, C₂₋₆-alkenyl, C₂₋₆-alkynyl, C₃₋₇-cycloalkyl, C₃₋₇-cycloalkyl-C₁₋₃-alkyl, C₃₋₇-cycloalkenyl, C₃₋₇-cycloalkenyl-C₁₋₃-alkyl, heterocyclyl, heterocyclyl-C₁₋₃-alkyl, aryl, aryl-C₁₋₃-alkyl, heteroaryl, or heteroaryl-C₁₋₃-alkyl,
wherein the above mentioned groups may optionally be substituted independently of one another by one or more groups selected from among fluorine, chlorine, bromine, iodine, hydroxy, carboxy, cyano, nitro, C₁₋₃-alkyl, C₁₋₃-alkoxy, C₁₋₃-alkyl-S, aryl, heteroaryl, heteroaryl-C₁₋₃-alkyl, aryl-C₁₋₃-alkyl, (R¹²)₂N—SO₂, (R¹²)₂N, (R¹²)₂N—C₁₋₃-alkyl- and (R¹²)₂N—CO—.

21. Compounds according to claim 1, characterised in that
R⁵ denotes C₁₋₆-alkyl, cyclopropyl, C₃₋₆-cycloalkyl-C₁₋₃-alkyl or phenyl-C₁₋₃-alkyl, wherein the above mentioned groups may optionally be substituted independently of one another by one or more groups selected from among fluorine, chlorine, bromine, iodine, cyano, hydroxy, carboxy, C₁₋₄-alkoxy- and (R¹²)₂N—.

22. Compounds according to claim 1, characterised in that
R⁵ denotes C₁₋₄-alkyl or cyclopropyl,
wherein one or more hydrogen atoms of the above mentioned groups may optionally be replaced by fluorine atoms.

23. Compounds according to claim 1, characterised in that
R⁶ denotes hydrogen, C₁₋₆-alkyl, C₂₋₆-alkenyl, C₂₋₆-alkynyl, C₃₋₇-cycloalkyl, C₃₋₇-cyclo alkyl-C₁₋₃-alkyl, C₃₋₇-cycloalkenyl, C₃₋₇-cyclo alkenyl-C₁₋₃-alkyl, heterocyclyl, heterocyclyl-C₁₋₃-alkyl, aryl, aryl-C₁₋₃-alkyl, heteroaryl or heteroaryl-C₁₋₃-alkyl,
wherein the above mentioned groups may optionally be substituted independently of one another by one or more groups selected from among fluorine, chlorine, bromine, iodine, hydroxy, carboxy, cyano, nitro, C₁₋₃-alkyl, C₃₋₇-cycloalkyl, heterocyclyl, heterocyclyl-C₁₋₃-alkyl, aryl, aryl-C₁₋₃-alkyl, heteroaryl, heteroaryl-C₁₋₃-alkyl, (R¹²)₂N, (R¹²)₂N—C₁₋₃-alkyl, (R¹²)₂N—CO, (R¹²)₂N—CO—N(R¹²), (R¹²)₂N—SO₂, R¹³—O— and R¹³—O—C₁₋₃-alkyl.

24. Compounds according to claim 1, characterised in that
R⁶ denotes hydrogen, C₁₋₆-alkyl, C₂₋₆-alkenyl, C₂₋₆-alkynyl, C₃₋₆-cycloalkyl, C₃₋₆-cycloalkyl-C₁₋₃-alkyl, heterocyclyl, heterocyclyl-C₁₋₃-alkyl, phenyl, phenyl-C₁₋₃-alkyl, heteroaryl or heteroaryl-C₁₋₃-alkyl,
wherein by the above-mentioned heteroaryl groups are meant 5- or 6-membered aromatic heteroaryl groups which contain 1, 2 or 3 heteroatoms selected from among N, O and S and
wherein the above mentioned groups may optionally be substituted independently of one another by one or more groups selected from among fluorine, chlorine, bromine, carboxy, hydroxy, cyano, C₁₋₃-alkyl, C₁₋₃-alkoxy, C₁₋₃-alkoxy-C₁₋₃-alkyl, hydroxy-C₁₋₃-alkyl, C₃₋₇-cycloalkyl, heterocyclyl, heterocyclyl-C₁₋₃-alkyl, aryl, (R¹²)₂N, (R¹²)₂N—C₁₋₃-alkyl, (R¹²)₂N—CO—N(R¹²)— and (R¹²)₂N—SO₂—.

25. Compounds according to claim 1, characterised in that
R⁶ denotes hydrogen, C₁₋₆-alkyl, C₃₋₆-cycloalkyl, C₃₋₅-cycloalkyl-C₁₋₃-alkyl, phenyl-C₁₋₃-alkyl or tetrahydropyranyl-C₁₋₃-alkyl,
wherein the above mentioned groups may optionally be substituted independently of one another by one or more groups selected from among fluorine, pyrrolidin-1-ylmethyl, hydroxy, cyano, C₁₋₃-alkyl, C₁₋₃-alkoxy, hydroxy-C₁₋₃-alkyl, H₂N, H₂N—C₁₋₃-alkyl, H₂N—CO—NH— and H₂N—SO₂—.

26. Compounds according to claim 1, characterised in that
R⁷ denotes hydrogen or C₁₋₄-alkyl,
wherein one or more hydrogen atoms of the C₁₋₄-alkyl group may be replaced by fluorine.

27. Compounds according to claim 1, characterised in that
R⁸ denotes heterocyclyl, heterocyclyl-C₁₋₃-alkyl, C₃₋₇-cycloalkenyl, aryl-C₁₋₃-alkyl, heteroaryl, heteroaryl-C₁₋₃-alkyl, R¹³—O, R¹³—O—C₁₋₃-alkyl, R¹⁰—SO₂—(R¹¹)N or R¹⁰—CO—(R¹¹)N,
wherein the above mentioned groups may optionally be substituted independently of one another by one or more groups selected from among C₁₋₄-alkyl, fluorine, chlorine, bromine, hydroxy, oxo, carboxy, cyano, nitro, C₃₋₇-cycloalkyl, heterocyclyl, (R¹²)₂N, (R¹²)₂N—CO, R¹³—CO, R¹³—O—CO, R¹²—CO—(R¹²)N, (R¹²)₂N—CO—(R¹²)N, (R¹²)₂N—SO₂, (R¹²)₂N—SO₂—(R¹²)N, R¹²—SO₂, R¹³—O, C₁₋₄-alkyl-S, F₃C, HF₂C, FH₂C, F₃C—O, HF₂C—O, FH₂C—O— and R¹²—SO₂—(R¹²)N—, and
R⁹ in each case independently of one another denote hydrogen, fluorine, chlorine, bromine, methyl, F₂HC, FH₂C or F₃C.

28. Compounds according to claim 1, characterised in that
R⁸ denotes C₁₋₄-alkoxy, C₃₋₆-cycloalkyl-oxy, C₃₋₆-cycloalkyl-C₁₋₃-alkoxy, R¹⁰—SO₂—(R¹¹)N or R¹⁰—CO—(R¹¹)N,
wherein the above mentioned groups may optionally be substituted independently of one another by one or more groups selected from among fluorine, chlorine, bromine, carboxy, cyano, C₁₋₃-alkyl, C₁₋₃-alkoxy, C₁₋₄-alkyl-S, R¹³—CO, R¹³—O—CO, R¹²—SO₂, F₃C, HF₂C, FH₂C, F₃C—O, HF₂C—O, FH₂C—O— and (R¹²)₂N—CO—, and
R⁹ in each case independently of one another denote hydrogen, fluorine, chlorine or bromine.

29. Compounds according to claim 1, characterised in that
R⁸ denotes R¹⁰—SO₂—(R¹¹)N or R¹⁰—CO—(R¹¹)N and
R⁹ in each case independently of one another denote hydrogen, fluorine, chlorine or bromine.

30. Compounds according to claim 1, characterised in that
R¹⁰ denotes C₂₋₆-alkenyl, C₂₋₆-alkynyl, C₃₋₇-cycloalkyl-C₁₋₃-alkyl, heterocyclyl, heterocyclyl-C₁₋₃-alkyl, C₃₋₇-cycloalkenyl, C₃₋₇-cycloalkenyl-C₁₋₃-alkyl, heteroaryl, heteroaryl-C₁₋₃-alkyl or (R¹²)₂N,
wherein the above mentioned groups may optionally be substituted by one or more groups selected from among fluorine, chlorine, bromine, hydroxy, carboxy, cyano, nitro, C₁₋₃-alkyl, heterocyclyl, heterocyclyl-C₁₋₃-alkyl, C₁₋₃-alkoxy, hydroxy-C₁₋₃-alkyl, R¹²—CO(R¹²)N, R¹²—SO₂(R¹²)N, (R¹²)₂N, (R¹²)₂N—C₁₋₃-alkyl- and (R¹²)₂N—CO—, and
R¹¹ denotes C₁₋₆-alkyl, C₂₋₆-alkenyl, C₂₋₆-alkynyl, C₃₋₇-cycloalkyl-C₁₋₃-alkyl, aryl, aryl-C₁₋₃-alkyl, heterocyclyl, heterocyclyl-C₁₋₃-alkyl, heteroaryl or heteroaryl-C₁₋₃-alkyl,
wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N— and (R¹²)₂N—C₁₋₃-alkyl.

31. Compounds according to claim 1, characterised in that
R¹⁰ denotes heterocyclyl, heteroaryl, heteroaryl-C₁₋₃-alkyl or (R¹²)₂N,
wherein by the above mentioned heteroaryl groups are meant 5- or 6-membered aromatic heteroaryl groups which contain 1, 2 or 3 heteroatoms selected from among N, O and S and
wherein the above mentioned groups may optionally be substituted independently of one another by one or more groups selected from among fluorine, chlorine, bromine, hydroxy, cyano, C₁₋₃-alkyl, C₁₋₃-alkoxy, heterocyclyl, heterocyclyl-C₁₋₃-alkyl, hydroxy-C₁₋₃-alkyl, (R¹²)₂N— and (R¹²)₂N—C₁₋₃-alkyl, and
R¹¹ denotes C₁₋₆-alkyl, heterocyclyl, heterocyclyl-C₁₋₃-alkyl, C₃₋₆-cycloalkyl-C₁₋₃-alkyl, phenyl, phenyl-C₁₋₃-alkyl, heteroaryl or heteroaryl-C₁₋₃-alkyl,
while by the above-mentioned heteroaryl groups are meant 5- or 6-membered aromatic heteroaryl groups which contain 1, 2 or 3 heteroatoms selected from among N, O and S and
wherein the above mentioned groups may optionally be substituted independently of one another by one or more groups selected from among fluorine, chlorine, bromine, hydroxy, cyano, C₁₋₃-alkyl, C₁₋₃-alkoxy, hydroxy-C₁₋₃-alkyl, heterocyclyl, heterocyclyl-C₁₋₃-alkyl, (R¹²)₂N— and (R¹²)₂N—C₁₋₃-alkyl.

32. Compounds according to claim 1, characterised in that
R¹⁰ denotes morpholinyl, piperidinyl, 4-methylpiperidinyl, pyrrolidinyl, pyridyl- and (CH₃)₂N,
wherein the above mentioned groups may optionally be substituted independently of one another by one or more groups selected from among fluorine, chlorine and bromine,
R¹¹ denotes methyl, ethyl, phenyl or 4-fluorophenyl,
wherein the above mentioned groups may optionally be substituted independently of one another by one or more groups selected from among fluorine, chlorine and bromine.

33. Compounds according to claim 1, characterised in that
R¹⁰ and R¹¹ together form a C₂₋₆-alkylene bridge, so that a heterocyclic ring is formed with the inclusion of the nitrogen atom linked to R¹¹ and the SO₂— or CO— group linked to R¹⁰,
wherein one or two —CH₂ groups of the C₂₋₆-alkylene bridge may be replaced independently of one another by O, S or —N(R¹²)— such that in each case two O, S or N atoms or an O and an S atom are not directly connected to one another, and
wherein the C atoms of the above mentioned C₂₋₆-alkylene bridge may optionally be substituted independently of one another by one or more groups selected from among fluorine, hydroxy, carboxy, F₃C, C₁₋₃-alkyl- and C₁₋₃-alkoxy.

34. Compounds according to claim 1, characterised in that
R¹⁰ and R¹¹ with the inclusion of the nitrogen atom bound to R¹¹ and the SO₂— group bound to R¹⁰, together form a heterocyclic ring of formulae (IIa), (IIb), (IIc) or (IId)

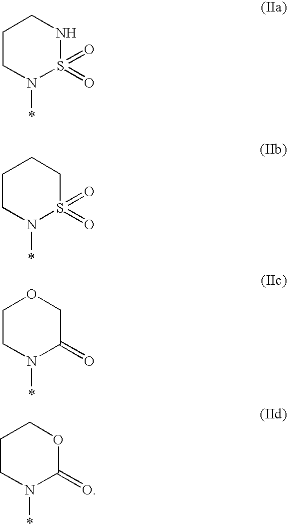

35. Compounds according to claim 1, characterised in that
R¹² denotes hydrogen or a C₁₋₆-alkyl group,
wherein one or more hydrogen atoms of the C₁₋₆-alkyl group may be replaced by fluorine.

36. Compounds according to claim 1, characterised in that
R¹³ denotes hydrogen or a C₁₋₃-alkyl group,
wherein one or more hydrogen atoms of the C₁₋₃-alkyl group may be replaced by fluorine.

37. Compounds according to claim 1 selected from among the formulae (Ia), (Ib), (Ic), (Id) or (Ie), 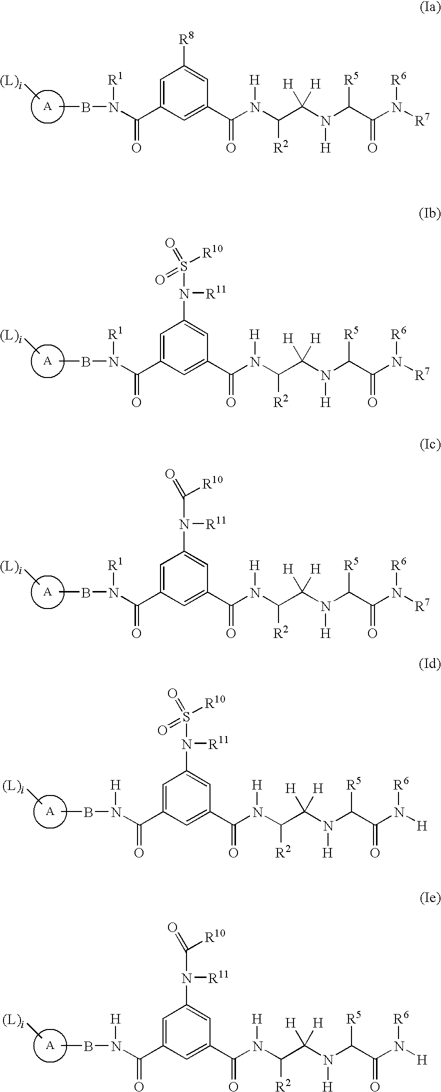
wherein
A, B, L, i, R¹, R², R³, R⁴, R⁵, R⁶, R⁷, R⁸, R⁹, R¹⁰, R¹¹, R¹² and R¹³ have the meanings given in the preceding claims.

38. Physiologically acceptable salts of the compounds according to claim 1.

39. Use of a compound according to claim 1 as a medicament.

40. Pharmaceutical composition, containing a compound according to claim 1 optionally together with one or more inert carriers and/or diluents.

41. Pharmaceutical composition according to claim 40, containing one or more medicinally effective active substances selected from among beta-secretase inhibitors, gamma-secretase inhibitors, amyloid aggregation inhibitors, directly or indirectly acting neuroprotective substances, antioxidants, Cox inhibitors, NSAIDs with additionally or only Aβ lowering properties; HMG-CoA reductase inhibitors, acetylcholinesterase inhibitors, NMDA receptor antagonists, AMPA agonists; substances that modulate the concentration or release of neurotransmitters, substances that induce the secretion of growth hormone, CB-1 receptor antagonists or inverse agonists, antibiotics, PDE-IV inhibitors, PDE-IX inhibitors, GABA_{A} inverse agonists, nicotine agonists, histamine H3 antagonists, 5 HT-4 agonists or partial agonists, 5HT-6 antagonists, a2-adrenoreceptor antagonists, muscarinic M1 agonists, muscarinic M2 antagonists and metabotropic glutamate-receptor 5 positive modulators.

42. Pharmaceutical composition according to claim 40, containing one or more medicinally effective active substances selected from among Alzhemed, Vitamin E, ginkgolides, donepezil, rivastigmine, tacrine, galantamine, memantine, NS-2330, ibutamoren mesylate, capromorelin, minocycline and rifampicin.

43. Use of at least one compound according to claim 1 as a β-secretase inhibitor.

44. Use of at least one compound according to claim 1 or a pharmaceutical composition thereof for preparing a medicament which is suitable for the treatment or prevention of diseases or conditions that are associated with abnormal processing of Amyloid Precursor Protein (APP) or aggregation of Abeta peptide.

45. Use of at least one compound according to claim 1 or a pharmaceutical composition thereof for preparing a medicament which is suitable for the treatment or prevention of diseases or conditions that can be influenced by inhibiting the β-secretase activity.

46. Use of at least one compound according to claim 1 or a pharmaceutical composition thereof for preparing a medicament for the treatment or prevention of Alzheimer's disease (AD), MCI (“mild cognitive impairment”), trisomy 21 (Down's syndrome), cerebral amyloidangiopathy, degenerative dementias, hereditary cerebral haemorrhage with amyloidosis-Dutch type (HCHWA-D), Alzheimer's dementia with Lewy bodies, trauma, stroke, pancreatitis, inclusion body myositis (IBM), as well as peripheral amyloidoses, diabetes or arteriosclerosis.

47. Use of at least one compound according to claim 1 or a pharmaceutical composition thereof for preparing a medicament for the treatment or prevention of Alzheimer's disease (AD).

48. Method of inhibiting β-secretase activity, characterised in that β-secretase is brought into contact with an inhibitory amount of a compound according to claim 1.
